(19) United States
(12) Patent Application Publication
(10) Pub. No.: US 2014/0140986 A1
(43) Pub. Date: May 22, 2014

(54) MATERIALS AND METHODS FOR DIRECTING AN IMMUNE RESPONSE TO AN EPITOPE

(71) Applicants: Carlos F. Santos, Tampa, FL (US); Mihaela Popa-Mckiver, Valrico, FL (US); Amy M. Mccord, New Port Richey, FL (US); Mark Hirschel, Minneapolis, MN (US)

(72) Inventors: Carlos F. Santos, Tampa, FL (US); Mihaela Popa-Mckiver, Valrico, FL (US); Amy M. Mccord, New Port Richey, FL (US); Mark Hirschel, Minneapolis, MN (US)

(73) Assignee: BIOVEST INTERNATIONAL, INC., TAMPA, FL (US)

(21) Appl. No.: 13/884,177

(22) PCT Filed: Nov. 8, 2011
(86) PCT No.: PCT/US11/59806

§ 371 Date: Sep. 23, 2013

Related U.S. Application Data
(60) Provisional application No. 61/411,459, filed on Nov. 8, 2010.
(60) Provisional application No. 61/420,233, filed on Dec. 6, 2010.

(51) Int. Cl. A61K 39/395; A61K 39/00

(52) U.S. Cl. CPC A61K 39/395; A61K 39/00
USPC 424/130.1

(57) ABSTRACT

The present invention relates to compositions, kits, and methods useful for directing an immune response to an epitope of an antigen in a subject, by sensitizing the subject to the epitope and/or by tolerizing the subject to the epitope. The sensitizing method comprises co-administering to the subject the epitope and an immunoglobulin M (IgM) constant region (IgM Fc region). The tolerizing method comprises co-administering to the subject the epitope and an immunoglobulin G (IgG) constant region (IgG Fc region) to the subject.

### CROSS-REFERENCE TO RELATED APPLICATIONS

This application claims the benefit of U.S. Provisional Application Ser. No. 61/420,233, filed Dec. 6, 2010, and U.S. Provisional Application Ser. No. 61/411,459, filed Nov. 8, 2010, the disclosure of each of which is incorporated herein by reference in its entirety, including all figures, tables, amino acid and nucleic acid sequences.

### BACKGROUND OF THE INVENTION

The modulation of immune responses targeting specific molecules remains the goal of a wide range of treatments for infections, malignancies, autoimmunity, transplant rejection, allergies and/or rejection of medical devices. Natural means of inducing specific responses to infectious disease are well known, and include preventive immunizations which confer lifelong immunity to unwanted pathogens. Other therapies, such as immunosuppression or induction of adaptive tolerance, seek to eliminate undesirable immune responses against “self” proteins that lead to disease.

In the treatment of cancer, for example, the induction of a tumor-specific immune response has long been sought by clinical medicine as a means for eliminating the tumor cells responsible for the disease while sparing healthy non-tumor cells. This can be achieved by immunizing the patient against specific molecules, or antigenic epitopes, which are present primarily on the cancer cell but absent (or expressed to a much lower degree) on normal cells in the body. Examples of such antigens are numerous and include such antigens as the surface idiotype (Id) of a malignant lymphoma, glycosphingolipid GD2, cell surface receptors such as ErbB2, which are abnormally found on breast cancer cells, etc.

A major challenge with using tumor material for immunization is that tumor antigens are typically weakly immunogenic. So-called tumor-associated antigens or tumor antigens are typically self-proteins to which the immune system has been conditioned against destroying. These antigens contain epitopes, or structural features which are capable of being specifically recognized and targeted by antibodies and lymphocytes in a patient if presented in the appropriate manner to the patient's immune system. The targeting of specific disease epitopes by idiotype vaccination has historically involved isolation or reconstitution of the tumor-specific epitope in conjunction with an immunoglobulin constant region (Fc). To date, however, no differing immune response or clinical outcomes have been reported in clinical trials of idiotype vaccines presenting idiotypic antigens in conjunction with IgM isotype Fc versus IgG isotype Fc.

An anti-idiotype vaccine is a vaccine comprising an antibody that recognizes another antibody as the antigen and binds to it. Anti-idiotype vaccines can stimulate the body to produce antibodies against tumor cells. Anti-idiotype vaccines are antibodies directed to an antibody. For example, an anti-idiotype vaccine for a tetanus antigen would comprise an antibody (Ab2) which binds to an antibody (Ab1) specific for the tetanus antigen. The Ab2 antibody then generates an immune response similar to that generated by the antigen itself.

The variable regions of the surface immunoglobulin (Ig) on a B cell form a specific antigen-binding site that is unique to each Ig and contain molecular determinants, termed idiotype (Id), which can themselves be recognized as antigens. Since B-cell malignancies are clonal proliferations, the Ig variable regions on the tumor cells are distinct from other normal B cells. The idiotypic determinants of the surface Ig of a B-cell lymphoma can therefore serve as a tumor-specific antigen for therapeutic vaccine development.¹

In experimental animal models, immunization with tumor-derived Id protein induced the host's immune system to reject tumor cells bearing idiotypic antigens.^{2,3} Optimal induction of tumor-specific immune responses required conjugation of Id protein to keyhole limpet hemocyanin (KLH)⁴ and administration with granulocyte-monocyte colony-stimulating factor (GM-CSF) as an adjuvant.⁵ Kwak et al. first demonstrated immunogenicity of Id vaccines in lymphoma patients.⁶ Subsequent pilot studies of this vaccine formulation demonstrated feasibility but primarily induced humoral immune responses.^{7,8} A landmark National Cancer Institute (NCI) phase II study of FL patients vaccinated with autologous hybridoma-derived Id-KLH+GM-CSF in first complete remission (CR) after prednisone, doxorubicin, cyclophosphamide, and etoposide (PACE) combination chemotherapy demonstrated lymphoma-specific CD8⁺ T-cell responses in 95% of patients. Cellular immune responses correlated with molecular remissions, demonstrating the potential for elimination of minimal residual disease by vaccination.⁹

### BRIEF SUMMARY OF THE INVENTION

Tumor-derived idiotype (Id) protein conjugated to keyhole limpet hemocyanin (KLH) administered with granulocyte-monocyte colony-stimulating factor (GM-CSF) can induce follicular lymphoma (FL)-specific immune responses that target tumor-specific antigenic determinants within the tumor cell's unique immunoglobulin (Ig) variable region (Fv). While Fv idiotypic determinants serve as specific tumor antigens, preclinical evidence suggests that the isotype of the Ig constant region (Fc) may independently influence the immunogenicity of hybridoma-derived immunoglobulins. Whereas Ids that have switched to IgG were tolerogenic, Ids of their IgM progenitors were highly immunogenic (Reitan et al. Proc Natl Acad Sci USA, 2002). Thus, the present inventors examined the clinical impact of tumor Ig isotype on disease-free survival (DFS) within a prospective randomized double-blind placebo-controlled multicenter phase III study of patient-specific tumor heterohybridoma-derived Id vaccine in advanced stage previously untreated FL patients with a lymph node adequate for vaccine production.

Among 76 patients receiving Id vaccine, 36 received IgM-Id vaccines and 40 IgG-Id vaccines corresponding to their tumor Ig isotype. Of 41 patients receiving control, 25 had tumors with IgM isotype and 15 had tumors with IgG isotype; 1 patient had a tumor with mixed IgM/IgG isotypes. Among 36 patients with IgM tumor isotype receiving an IgM-Id vaccine, median time to relapse after randomization was 50.6 months, versus 27.1 months in the IgM tumor isotype control-treated patients (log-rank p=0.002; HR=0.36 (p=0.003); [95% CI: 0.19-0.71]) (shown in FIG. 2). Among 40 patients with IgG tumor isotype receiving an IgG-Id vaccine, median time to relapse after randomization was 35.1 months, versus 32.4 months in control-treated patients with IgG tumor isotype (log-rank p=0.807; HR=1.1 (p=0.807); [95% CI: 0.50-2.44]) (shown in FIG. 3). It must be noted that although this trial was not powered to address such subletting, the dramatically different results suggest that the treatment effect is different in the two groups, with a surprisingly small p-value of 0.085.

These results suggest that the isotype of an Id vaccine may influence DFS in FL patients vaccinated in first complete remission. Unexpectedly, it was observed that the IgM-Id vaccine significantly prolonged remission duration in comparison to IgG-Id vaccine. Compared to other phase III Id vaccine trials that used recombinant Id vaccines with IgG constant regions for all patients, the positive outcome of this study may reflect the use of hybridomas to produce Id protein with variable and constant regions identical to patient tumor Ig. Additional studies are expected to further evaluate the effect of Id vaccine isotype on clinical outcome in FL and other B-cell malignancies. These findings should have profound implications on strategies for the control and manipulation of immune responses, and for treatment and prevention of human disorders including, but not limited to, cancer (e.g., B-cell malignancies).

The invention concerns methods for directing an immune response to an epitope from an antigen in a subject. One aspect of the invention includes a method of sensitizing a subject to an epitope, comprising co-administering the epitope and an immunoglobulin M (IgM) constant region (IgM Fc region) to the subject. Another aspect of the invention includes a method of tolerizing a subject to an epitope, comprising co-administering the epitope and an immunoglobulin G (IgG) constant region (IgG Fc region) to the subject. Thus, both methods of the invention can be described as encompassed by the broader method for directing an immune response to an epitope from an antigen in a subject, comprising:
(a) sensitizing the subject to the epitope, comprising co-administering the epitope and an immunoglobulin M (IgM) constant region (IgM Fc region) to the subject; or
(b) tolerizing the subject to the epitope, comprising co-administering the epitope and an immunoglobulin G (IgG) constant region (IgG Fc region) to the subject.

In the methods of the invention, the antigen may be any molecule (for example, a polypeptide, nucleic acid molecule, carbohydrate, glycoprotein, lipid, lipoprotein, glycolipid, or small molecule) that is capable of eliciting an immune response and contains an epitope or antigenic determinant to which an immunoglobulin can specifically bind. In some embodiments, the antigen is an immunoglobulin (Ab2) directed against the idiotype of a monoclonal antibody (Ab1), wherein the Ab1 is directed against the antigen and the Ab2 mimics the antigen. In some embodiments, the antigen is a polypeptide, nucleic acid molecule, carbohydrate, glycoprotein, lipid, lipoprotein, glycolipid, or small molecule. In some embodiments, the antigen is selected from among a cancer antigen, autoantigen, endogenous antigen, infectious agent antigen, drug (small molecule) antigen, toxin, venom, biologic antigen, environmental antigen, transplant antigen, and implant antigen.

Whether the sensitizing of (a) or the tolerizing of (b) is carried out on the subject will depend upon the desired immune response to the epitope in question. Sensitizing a subject to an epitope can provide a therapy and/or prophylaxis of a disorder associated with the epitope or antigen. In contrast, tolerizing a subject to an epitope can bias the subject's immune system to elicit a reduced immune response to the epitope (relative to the immune response that may otherwise occur in the absence of tolerization).

In some embodiments, the sensitizing of (a) comprises administering a fusion polypeptide comprising the epitope and the IgM Fc region.

In some embodiments, the sensitizing of (a) comprises administering a nucleic acid molecule encoding the epitope and the IgM Fc region, and wherein the nucleic acid molecule is expressed in the subject to produce the epitope and the IgM Fc region separately or as a fusion polypeptide.

In some embodiments, the sensitizing of (a) comprises co-administering the epitope and the IgM Fc separately, in separate formulations or within the same formulation.

Optionally, in any embodiment, the sensitizing of (a) further comprises administering at least one immune adjuvant (for example, granulocyte-monocyte colony stimulating fragment (GM-CSF) or bovine serum albumin (BSA)) to the subject before, simultaneously with, or after co-administration of the epitope and IgM Fc region.

In some embodiments, in the sensitizing of (a), the epitope and the IgM Fc region are administered in conjunction with a carrier protein (for example, keyhole limpet hemocyanin (KLH)).

In some embodiments, the tolerizing of (b) comprises administering to the subject a fusion polypeptide comprising the epitope and the IgG Fc region.

In some embodiments, the tolerizing of (b) comprises administering a nucleic acid molecule encoding the epitope and the IgG Fc region, and wherein the nucleic acid molecule is expressed in the subject to produce the epitope and the IgG Fc region separately or as a fusion polypeptide.

In some embodiments, the tolerizing of (b) comprises co-administering the epitope and the IgG Fc separately, in separate formulations or in the same formulation.

Optionally, the tolerizing of (b) can further comprises administering a tolerizing agent. Examples of tolerizing agents include, but are not limited to, IVIG (intravenous immunoglobulin IgG) or an immunosuppressant.

In some embodiments, the tolerizing of (b) is carried out on the subject prior to transplantation, and wherein the antigen is an HLA antigen within the donor, and is selected from among HLA-A, HLA-B, HLA-E, HKA-G, HLA-F, HLA-DR, HLA-DQ, HLA-DP.

In some embodiments, the subject has cancer, the antigen is a cancer antigen identified in the subject, the cancer is eliminated or attenuated following the sensitizing of (a), and the tolerizing of (b) is carried out after the cancer is eliminated or attenuated to reduce unwanted autoimmune reaction from the sensitizing of (a).

In some embodiments, the epitope is the epitope of a gene delivery vector, and the tolerizing of (b) is carried out prior to administration of the gene delivery vector to the subject. In these cases, the tolerizing method of the invention is useful in reducing undesired immune responses to epitope-bearing gene delivery vectors (for example, a viral vector or non-viral vector).

In some embodiments, in the tolerizing of (b), the epitope is the epitope of an implant to be introduced into the subject. The method may further comprise introducing the implant into the subject after the tolerizing of (b). This will be useful in tolerizing a subject to epitope-bearing implants. Such implants may include, for example, electrically powered implants (for example, artificial pacemakers), bioimplants (biomaterial surgically implanted in a subject's body to replace damaged tissue (for example, orthopedic reconstructive prosthesis), cardiac prostheses (artificial valves), skin, and cornea), dental implants, and orthopedic implants.

In some embodiments, the epitope comprises a mimotope. The mimotope may be produced by methods known in the art, such as phage display or anti-idiotypic antibody generation by immunization of an animal with a monoclonal antibody.

In some embodiments, the antigen is a tumor-associated antigen (TAA), and the TAA is a carbohydrate antigen having one or more post-translational modifications that differ from the wild-type protein, comprises a fusion region of a protein resulting from a gene fusion that is present in malignant cells but not present in non-malignant cells, and/or wherein the TAA comprises a receptor tyrosine kinase (RTK) that is deregulated and/or dysfunctional in tumor cells due to autocrine activation, chromosomal translocations, RTK overexpression, or gain-of-function mutations in the RTK gene or protein.

In some embodiments, the antigen is an antigen that is endogenous to the subject. For example, the endogenous antigen can be an aberrantly expressed polypeptide from among amyloid beta, alpha synuclein, cystatin C, tau, ABri, ADan, superoxide dismutase (SOD), mutant Huntington, PrP^{Sc}, or a fragment of any of the foregoing.

In some embodiments, the antigen is an immunoglobulin expressed by a B-cell malignancy. Alternatively, in some embodiments, the antigen is not an immunoglobulin expressed by a B-cell malignancy (for example, in some embodiments, the antigen is not an autologous idiotype vaccine). In some embodiments, the antigen is not an immunoglobulin.

In some embodiments, in the sensitizing of (a), the subject has cancer and, prior to the sensitizing of (a), the subject undergoes therapy for the cancer (for example, chemotherapy, immunotherapy, radioimmunotherapy, radiation therapy, surgery, or a combination of two or more of the foregoing). For example, if the subject has a tumor, the subject may be treated prior to sensitization, to reduce or eliminate the tumor. In some embodiments, the cancer is a B-cell malignancy, and the antigen is an immunoglobulin expressed by the B-cell malignancy. Alternatively, in some embodiments, the antigen is not an immunoglobulin expressed by a B-cell malignancy (for example, in some embodiments, the antigen is not an autologous idiotype vaccine). In some embodiments, the antigen is not an immunoglobulin.

In the methods of the invention, the subject may be a human or non-human animal. In some embodiments, the subject is a human or non-human mammal. Preferably, the subject is human. The subject may be any age (for example, infant, child, adolescent, adult, elderly adult). The subject may be any gender.

In the methods and compositions of the invention, the IgM Fc region and IgG Fc region may be Fc regions of human or humanized immunoglobulins, and may be recombinant or non-recombinant (recombinantly produced or non-recombinantly produced). Preferably, in cases in which the subject is human, the Fc region utilized is human or humanized.

The methods described herein can be performed, e.g., by utilizing compositions and pre-packaged kits of the invention. Thus, another aspect of the invention is a composition comprising an epitope; and an immunoglobulin M (IgM) constant region (IgM Fc region) or an immunoglobulin G (IgG) constant region (IgG Fc region). In some embodiments, the composition further comprises an immunomodulatory agent.

In some embodiments, the composition comprises an epitope, an IgM Fc region, and an adjuvant. In some embodiments, the composition comprises an epitope, an IgM Fc region, and a T-regulatory cell inhibitor. In some embodiments, the composition comprises an epitope, an IgG Fc region, and an immunosuppressive agent.

Another aspect of the invention is a kit for sensitizing a subject to an epitope, wherein the kit comprises at least one IgM Fc region and printed instructions for sensitizing a subject to an epitope using the IgM Fc region. In some embodiments, the sensitizing kit further comprises an epitope, adjuvant, carrier protein, an assay for an immune response, or any combination of two or more of the foregoing.

Another aspect of the invention is a kit for tolerizing a subject to an epitope, wherein the kit comprises at least one IgG Fc region and printed instructions for tolerizing a subject to an epitope. In some embodiments, the tolerizing kit further comprises an epitope, adjuvant, carrier protein, an assay for T-regulatory cell number and/or activity, an assay for immune response, or any combination of two or more of the foregoing.

Another aspect of the invention is a kit for sensitizing or tolerizing a subject to an epitope, wherein the kit comprises at least one IgM Fc region, at least one IgG Fc region, printed instructions for sensitizing a subject to an epitope using the IgM Fc region, and printed instructions for tolerizing a subject to an epitope using the IgM Fc region. In some embodiments, the sensitizing/tolerizing kit further comprises an epitope, adjuvant, carrier protein, assay for an immune response, assay for T-regulatory cell number and/or activity, or any combination of two or more of the foregoing.

### BRIEF DESCRIPTION OF THE FIGURES

FIGS. 1A and 1B are, respectively, a clinical trial schema and flow chart of enrollment, randomization, and treatment. As shown in FIG. 1A, advanced stage, previously untreated, follicular lymphoma patients underwent a lymph node biopsy (LN Bx) after enrollment and were treated with prednisone (60 mg/m2 orally daily on days 1 to 14), doxorubicin (25 mg/m2 IV on days 1 and 8), cyclophosphamide (650 mg/m2 IV on days 1 and 8), and etoposide (120 mg/m2 IV on days 1 and 8) (PACE) chemotherapy every 28 days. Patients achieving a complete response (CR)/complete response unconfirmed (CRu) were stratified according to International Prognostic Index (IPI) and number of chemotherapy cycles and randomized 2:1 to receive five injections of the Id-vaccine (Id-KLH+GM-CSF) or control vaccine (KLH+GM-CSF), respectively. As shown in FIG. 1B, two hundred thirty-four patients were enrolled and 117 patients were randomized to receive at least one dose of the blinded vaccine; 76 received Id-vaccine and 41 received control vaccine. Patients receiving fewer than 5 immunizations either withdrew from the study† or relapsed‡ before completion.

FIGS. 2A and 2B are graphs showing disease-free survival (DFS) and overall survival (OS) according to treatment group for the randomized patients that received blinded vaccinations (N=117). Kaplan-Meier survival curves for DFS (FIG. 2A) and OS (FIG. 2B) for randomized patients who received at least one dose of Id-vaccine (N=76; red) or control vaccine (N=41; blue) are shown. The number of events, median, and 95% confidence intervals for each group are also presented.

FIGS. 3A and 3B are graphs showing DFS according to tumor immunoglobulin (Ig) heavy chain isotype for the randomized patients that received blinded vaccination. Randomized patients who received at least one dose of the Id-vaccine or control vaccine were grouped according to the isotype of their tumor Ig heavy chain. Kaplan-Meier survival curves for DFS for Id-vaccine (red) and control vaccine (blue) groups according to IgM (FIG. 3A) and IgG (FIG. 3B) isotype are shown. The number of events, median DFS, and 95% confidence intervals for each group are also presented.

FIG. 4 is a graph showing DFS according to treatment group for all randomized patients (N=177). Kaplan-Meier survival curves for DFS for all randomized patients are shown according to treatment group: Id-vaccine (N=118; red); control (N=59; blue). The number of events, median DFS, and 95% confidence intervals for each group are also presented.

FIG. 5 is a graph showing DFS according to treatment group for randomized patients who did not receive vaccination (N=60). Kaplan-Meier survival curves for DFS for randomized patients who did not receive vaccination are shown according to treatment group: Id-vaccine (N=42; red); control (N=18; blue). The number of events, median DFS, and 95% confidence intervals for each group are also presented.

FIG. 6 is a graph showing DFS for the randomized patients that received IgM-Id vaccine versis all controls. Kaplan-Meier survival curves for DFS for the IgM-Id vaccinated patients (N=35; red) and all patients in the control arm (N=41; blue) are shown. The number of events, median DFS, and 95% confidence intervals for each group are also presented.

### DETAILED DESCRIPTION OF THE INVENTION

Vaccination with hybridoma-derived, autologous tumor immunoglobulin (Id) conjugated to keyhole limpet hemocyanin (KLH) and administered with granulocyte-monocyte colony-stimulating factor (GM-CSF) induces follicular lymphoma (FL)-specific immune responses. To determine the clinical benefit of this vaccine, a double-blind multicenter controlled phase III trial was conducted. Advanced stage, treatment-naive FL patients achieving complete response (CR) or complete response unconfirmed (CRu) after chemotherapy were randomized 2:1 to receive either Id-vaccine (Id-KLH+GM-CSF) or control (KLH+GM-CSF). Primary efficacy endpoints were disease-free survival (DFS) for (1) all randomized patients; (2) randomized patients receiving at least one dose of Id-vaccine or control. Of 234 patients enrolled, 177 (81%) achieved CR/CRu following chemotherapy and were randomized. For 177 randomized patients, including 60 patients not vaccinated due to relapse (n=55) or other reasons (n=5), median DFS between Id-vaccine and control arms was 23.0 vs. 20.6 months, respectively (P=0.256; HR=0.81; 95% CI:0.56-1.16). For 117 patients who received Id-vaccine (n=76) or control (n=41), median DFS after randomization was 44.2 months for the Id-vaccine arm versus 30.6 months for the control arm (P=0.047; HR=0.62; 95% CI:0.39-0.99) at median follow-up of 56.6 months (range 12.6-89.3). Median DFS was significantly prolonged for patients receiving IgM-Id (52.9 versus 28.7 months; p=0.001) but not IgG-Id vaccine (35.1 versus 32.4 months; p=0.807) compared to immunoglobulin heavy chain isotype matched control-treated patients.

The invention concerns methods for directing an immune response to an epitope from an antigen in a subject. One aspect of the invention includes a method of sensitizing a subject to an epitope, comprising co-administering the epitope and an immunoglobulin M (IgM) constant region (IgM Fc region) to the subject. Another aspect of the invention includes a method of tolerizing a subject to an epitope, comprising co-administering the epitope and an immunoglobulin G (IgG) constant region (IgG Fc region) to the subject. Thus, both methods of the invention can be described as encompassed by the broader method for directing an immune response to an epitope from an antigen in a subject, comprising:
(a) sensitizing the subject to the epitope, comprising co-administering the epitope and an immunoglobulin M (IgM) constant region (IgM Fc region) to the subject; or
(b) tolerizing the subject to the epitope, comprising co-administering the epitope and an immunoglobulin G (IgG) constant region (IgG Fc region) to the subject.

The selection of epitope(s), and the selection of IgM or IgG Fc region will depend upon the objective, i.e., the desired immune response and whether sensitization to the antigen or tolerization to the antigen is the goal.

The methods described herein can be performed, e.g., by utilizing compositions and pre-packaged kits of the invention. Thus, another aspect of the invention is a composition comprising an epitope; and an immunoglobulin M (IgM) constant region (IgM Fc region) or an immunoglobulin G (IgG) constant region (IgG Fc region). In some embodiments, the composition further comprises an immunomodulatory agent.

In some embodiments, the composition comprises an epitope, an IgM Fc region, and an adjuvant. In some embodiments, the composition comprises an epitope, an IgM Fc region, and a T-regulatory cell inhibitor. In some embodiments, the composition comprises an epitope, an IgG Fc region, and an immunosuppressive agent.

Another aspect of the invention is a kit for sensitizing a subject to an epitope, wherein the kit comprises at least one IgM Fc region and printed instructions for sensitizing a subject to an epitope using the IgM Fc region. In some embodiments, the sensitizing kit further comprises an epitope, adjuvant, carrier protein, assay for immune response, or any combination of two or more of the foregoing.

Another aspect of the invention is a kit for tolerizing a subject to an epitope, wherein the kit comprises at least one IgG Fc region and printed instructions for tolerizing a subject to an epitope. In some embodiments, the tolerizing kit further comprises an epitope, adjuvant, carrier protein, assay for an immune response, assay for T-reg cell level and/or activity, or any combination of two or more of the foregoing.

Another aspect of the invention is a kit for sensitizing or tolerizing a subject to an epitope, wherein the kit comprises at least one IgM Fc region, at least one IgG Fc region, printed instructions for sensitizing a subject to an epitope using the IgM Fc region, and printed instructions for tolerizing a subject to an epitope using the IgM Fc region. In some embodiments, the sensitizing/tolerizing kit further comprises an epitope, adjuvant, carrier protein, or any combination of two or more of the foregoing.

The kits of the invention can be used for sensitization (including, for example, an assay for immune response, an IgM Fc vaccine, adjuvant, etc.) or tolerization (an assay for T-reg cell level, monitoring of a subject's immune response to sensitizing antigen, which can be measured by methods known in the art (e.g., ELISA) over the course of treatment (for example, looking for a lower immune response and achieving the T-reg cell level threshold before stopping tolerization, looking for the presence of T-reg cells and/or activity which should be induced (e.g., higher numbers of CD4+cd25HIFoxp3+ cells by flow cytometry) by tolerization)).

Kits of the invention may comprise packaging and containers or receptacles for containing each component of the kit. The kits can also contain a solid support such as microtiter multi-well plates, standards, assay diluent, wash buffer, adhesive plate covers, and/or instructions for carrying out a method of the invention using the kit. If a biological sample is to be obtained (such as for an assay for an immune response, or an assay for T-reg cell level and/or activity), the kit can include means for obtaining a biological sample (such as a needle for venipuncture) and/or one or more protease inhibitors (e.g., a protease inhibitor cocktail) to be applied to the biological sample to be assayed (such as blood).

### I. SENSITIZATION

In one aspect, the invention features a method for sensitizing a subject to an epitope from an antigen, thereby enhancing (inducing or increasing) humoral and/or cellular immunoreactivity to the antigen, the method comprising co-administering the epitope and an immunoglobulin M (IgM) constant region (IgM Fc region) to the subject. Sensitization may be induced to one or more epitopes of single antigen, or epitopes of multiple antigens, and sensitization to an epitope may exist at the B cell level or T cell level or at both levels. In some embodiments, prior to sensitization, the method further comprises identifying the subject as one in need of sensitization to the epitope.

In some embodiments, sensitization of the subject comprises administering a fusion polypeptide comprising both the epitope and the IgM Fc region.

In some embodiments, sensitization of the subject comprises administering a nucleic acid molecule encoding the epitope and the IgM Fc region, such that the nucleic acid molecule is expressed to produce the epitope and the IgM Fc region separately or as a fusion polypeptide.

In some embodiments, sensitization of the subject comprises co-administering the epitope and the IgM Fc separately, in separate formulations or in the same formulation.

Optionally, in any embodiment of the sensitization method, sensitization of the subject may further comprise administering at least one immune adjuvant (for example, granulocyte-monocyte colony stimulating fragment (GM-CSF) or bovine serum albumin (BSA)) to the subject before, simultaneously with, or after co-administration of the epitope and IgM Fc region.

Optionally, in any embodiment of the sensitization method, the epitope and the IgM Fc region can be administered in conjunction with a carrier protein (for example, keyhole limpet hemocyanin (KLH)).

### II. TOLERIZATION

In another aspect, the invention features a method for tolerizing a subject to an epitope from an antigen, thereby reducing (lessening or eliminating) humoral and/or cellular immunoreactivity to the antigen, the method comprising co-administering the epitope and an immunoglobulin G (IgG) constant region (IgG Fc region) to the subject. Without being limited by theory, it is proposed that tolerization occurs through suppression of: effector T cell response, helper T cell response, B cell response, or a combination of two or more of the foregoing. Tolerance may be induced to all epitopes or only some epitopes on an antigen and tolerance to a single antigen may exist at the B cell level or T cell level or at both levels. Induction of immunologic tolerance in accordance with the invention can be useful in treatment and/or prophylaxis of various disorders that involve an undesirable immune response to an epitope, for example, in preventing or delaying onset of rejection of cells, tissues, or organs (for example, organ allografts and xenografts), treating autoimmune disorders, and treating allergic diseases. In some embodiments, prior to tolerization, the method further comprises identifying the subject as one in need of tolerization to the epitope.

In some embodiments, the tolerizing of (b) comprises administering to the subject a fusion polypeptide comprising the epitope and the IgG Fc region.

In some embodiments, the tolerizing of (b) comprises administering a nucleic acid molecule encoding the epitope and the IgG Fc region, and wherein the nucleic acid molecule is expressed to produce the epitope and the IgG Fc region separately or as a fusion polypeptide.

In some embodiments, the tolerizing of (b) comprises co-administering the epitope and the IgG Fc separately, in separate formulations or in the same formulation.

Optionally, the tolerizing of (h) can further comprise administering a tolerizing agent. Examples of tolerizing agents include, but are not limited to, IVIG (intravenous immunoglobulin IgG) or an immunosuppressant.

In some embodiments, the tolerizing of (b) is carried out on the subject prior to transplantation, and wherein the antigen is an HLA antigen within the donor, and is selected from among HLA-A, HLA-B, HLA-E, HKA-G, HLA-F, HLA-DR, HLA-DQ, HLA-DP.

In some embodiments, the epitope is the epitope of a gene delivery vector, and the tolerizing of (b) is carried out prior to administration of the gene delivery vector to the subject. In these cases, the tolerizing method of the invention is useful in reducing undesired immune responses to epitope-bearing gene delivery vectors (for example, a viral vector or non-viral vector).

### III. EPITOPES

The epitopes used in the compositions and methods of the invention are antigenic determinant sites on an antigen to which an immunogolublin (or antigen binding fragment thereof) can specifically bind. Epitopes can be formed both from contiguous amino acids or noncontiguous amino acids juxtaposed by tertiary folding of a protein. Epitopes can be found on the Fab (variable) region of immunoglobulins (referred to as “idiotypic determinants”) and comprise the immunoglobulin's “idiotype”.

Epitopes can be administered to a subject in isolation from an antigen or as part of an intact or modified antigen. The epitope and antigen may be naturally occurring or artificially produced. Depending on the nature of the epitope or antigen, the epitope or antigen may be isolated or purified from a matrix or substance of origin, synthesized, or recombinantly produced, for example.

Epitopes and antigens may be from a human or non-human animal, plant, bacteria, protozoan, parasite, virus, etc. In some embodiments, the antigen is a polypeptide, nucleic acid molecule, carbohydrate, glycoprotein, lipid, lipoprotein, glycolipid, or small molecule.

In some embodiments, the antigen is selected from among a cancer antigen, autoantigen, endogenous antigen, infectious agent antigen, drug (small molecule) antigen, toxin, venom, biologic antigen, environmental antigen, transplant antigen, and implant antigen.

In some embodiments, the epitope comprises a mimotope. The mimotope may be produced by methods known in the art, such as phage display (see, e.g., Pini, A. et al., “Design and use of a phage display library. Human antibodies with subnanomolar affinity against a marker of angiogenesis eluted from a two-dimensional gel,” J Biol Chem, 273(34):21769-76 (1998); Boel, E. et al., “Functional human monoclonal antibodies of all isotypes constructed from phage display library-derived single-chain Fv antibody fragments,” J Immunol Methods, 239(1-2): p. 153-66 (2000)) or anti-idiotypic antibody generation by immunization of an animal with a monoclonal antibody.

A. Epitopes of Cancer Antigens

The epitope used to sensitize or tolerize the subject can be a cancer antigen. In some embodiments, the antigen is a tumor-associated antigen. In some embodiments, the antigen is a tumor-specific antigen.

In some embodiments of the invention, the antigen is a tumor-associated antigen (TAA), and the TAA is a carbohydrate antigen having one or more post-translational modifications that differ from the wild-type protein, comprises a fusion region of a protein resulting from a gene fusion that is present in malignant cells but not present in non-malignant cells, and/or wherein the TAA comprises a receptor tyrosine kinase (RTK) that is deregulated and/or dysfunctional in tumor cells due to autocrine activation, chromosomal translocations, RTK overexpression, or gain-of-function mutations in the RTK gene or protein.

In some embodiments of the invention, the antigen is an immunoglobulin expressed by a B-cell malignancy. Examples of B-cell malignancies include, but are not limited to, non-Hodgkin's lymphoma, Hodgkin's lymphoma, chronic lymphocytic leukemia, mantle cell lymphoma and multiple myeloma. Additional B-cell malignancies include, for example. B-cell prolymphocytic leukemia, lymphoplasmocytic leukemia, splenic marginal zone lymphoma, marginal zone lymphoma (extra-nodal and nodal), plasma cell neoplasms (e.g., plasma cell myeloma, plasmacytoma, monoclonal immunoglobulin deposition diseases, heavy chain diseases), and follicular lymphoma (e.g., Grades I, II, III, or IV).

In some embodiments, in the sensitizing of (a), the subject has cancer and, prior to the sensitizing of (a), the subject undergoes therapy for the cancer (for example, chemotherapy, immunotherapy, radioimmunotherapy, radiation therapy, surgery, or a combination of two or more of the foregoing). In some embodiments, the cancer is a B-cell malignancy, and the antigen is an immunoglobulin expressed by the B-cell malignancy. Alternatively, in some embodiments, the antigen is not an immunoglobulin expressed by a B-cell malignancy. In some embodiments, the antigen is not an immunoglobulin.

In some embodiments, the tumor-associated antigen is derived from tumor cells obtained from the subject. In some embodiments, the tumor-associated antigen is one or more antigens selected from among 17-1A, 707-AP, AFP, Annexin II, ART-4, BAGE, BAGE-1, β-catenin, BCG, bcr/abl, Bcr/abl e14a2 fusion junction, bcr-abl (b3a2), bcr-abl (b3a2), bcr-abl p190 (e1a2), bcr-abl p210 (b2a2), bcr-abl p210 (b3a2), bcr-abl p210 (b3a2), bullous pemphigoid antigen-1, CA19-9, CA125, CA215, CAG-3, CAMEL, Cancer-testis antigen, Caspase-8, CCL3, CCL4, CD16, CD20, CD3, CD30, CD55, CD63, CDC27, CDK-4, CDR3, CEA, cluster 5, cluster-5A, cyclin-dependent kinase-4, Cyp-B, DAM-10, DAM-6, Dek-cain, E7, EGFR, EGFRvIII, EGP40, ELF2 M, EpCAM, FucGM1, G250, GA733, GAGE, GAGE-1-8, gastrin cancer associated antigen, GD2, GD3, globoH, glycophorin, GM1, GM2, GM3, GnTV, Gn-T-V, gp100, Her-2/neu, HERV-K-ME, high molecular weight-associated antigen, high molecular weight proteo-glycan (HMPG), HPV-16 E6, HPV-16 E7, HPVE6, HSP70-2M, HST-2, hTERT, human chorionic gonadotropin (HCG), Human milk fat globule (HMFG), iCE, KIAA0205, KK-LC-1, KM-HN-1, L6, LAGE-1, Lcose4Cer, LDLR/FUT, Lewis A, Lewis v/b, M protein, MAGE-1, MVC, MAGE-A1-12, MAGE-C2, MAHGE-3, MART-1/Melan-A, MC1R, ME491, MUC1, MUC2, mucin, MUM-1, MUM-2, MUM-3, mutated p53, Myosin, MZ2-E, N9 neuraminidase, NA88, NA88-A, nasopharyngeal carcinoma antigen, NGA, NK1/c-3, Novel bcr/ablk fusion BCR exons 1, 13, 14 with ABL exons 4, NY-ESO-1/LAGE-2, NY-ESO-1b, OC125, osteosarcoma associated antigen-1, P15, p190 mimor bcr-abl (e1a2), p53, Pm1/RARa, Polysialic acid, PRAME, PSA, PSM, RU1, RU2, SAGE, SART-1, SART-2, SART-3, Sialyl LeA, Sp17, SSX-2, SSX-4, surface immunoglobulin, TAG-1, TAG-2, TEL/AML1, TPI, TRAG-3, TRP-1 (gp75), TRP-2, TRP2-INT2, hTRT, tumor associated glycoprotein-72 (TAG-72), tyrosinase, u-PA, WT1, and XAGE-1b, or an immunogenic fragment of any of the foregoing antigens.

In some embodiments, the tumor associated antigen is identified by the SEREX (serological analysis of recombinant cDNA expression library) approach or based on the serological screening of cDNA expression library generated from tumor tissues of various origin or cancer cell lines, and identifying immunogenic tumor proteins based on their reactivity with autologous patient sera.

In some embodiments, the tumor-associated antigen is a carbohydrate antigen having one or more post-translational modifications that differ from the wild-type protein.

In some embodiments, the tumor-associated antigen comprises a fusion region of a protein resulting from a gene fusion that is resent in malignant cells but not present in non-malignant cells. In some embodiments, the tumor-associated antigen comprises a receptor tyrosine kinase that is deregulated and/or dysfunctional in tumor cells due to autocrine activation, chromosomal translocations, RTK overexpression, or gain-of-function mutations in the RTK gene or protein.

B. Epitopes of Infectious Agent Antigens

The epitope used to sensitize or tolerize the subject may be an epitope of an antigen of an infectious agent. The infectious agent may be pathogenic or non-pathogenic to the subject. For example, the antigen may be derived from a bacterial pathogen. In some embodiments, the bacterial pathogen is selected from among Acinetobacter baumannii (formerly Acinetobacter calcoaceticus), Actinobacillus, Actinomyces pyogenes (formerly Corynebacterium pyogenes), Actinomyces israelii, nocardia asteroids, N. brasiliensis, Aeromonas hydrophila, Amycolata autotrophica, Archanobacterium haemolyticum (formerly Corynebacterium haemolyticum), Arizona hinshawii—all serotypes, Bacillus anthracis, Bacteroides fragilis, Bartonella henselae, B. quintana, B. vinsonii, Bordetella including B. pertussis, Borrelia recurrentis, B. burgdorferi, Burkholderia (formerly Pseudomonas species) except those listed in BSL III), Campylobacter coli, C. fetus, C. jejuni, Chlamydia psittaci, C. trachomatis, C. pneumonia, Clostridium botulinum (neurotoxin producing species), Clostridium botulinum neurotoxins, Cl. chauvoei, Cl. haemolyticum, Cl. histolyticum, Cl. novyi, Cl. septicum, Cl. Tetani, Cl. Perfirngens epsilon toxin, Corynebacterium diphtheriae, C. pseudotuberculosis, C. renale, Dermatophilus congolensis, Edwardsiella tarda, Erysipelothrix rhusiopathiae, Escherichia coli—all enteropathogenic, enterotoxigenic, enteroinvasive and strains bearing K1 antigen, including E. coli O157:H7, Haemophilus ducreyi, H. influenzae, Helicobacter pylori, Klebsiella—all species except K. oxytoca (RG1), Legionella including L. pneumophila, Leptospira interrogans—all serotypes, Listeria, Moraxella, Mycobacterium (except those listed in BSL III) including M. avium complex, M. asiaticum, M. bovis BCG vaccine strain, M. chelonei, M. fortuitum, M. kansasii, M. leprae, M. malmoense, M. marinum, M. paratuberculosis, M. scrofulaceum, M. simiae, M. szulgai, M. ulcerans, M. xenopi, Mycoplasma, Neisseria gonorrhoeae, N. meningitides, Nocardia asteroides, N. brasiliensis, N. otitidiscaviarum, N. transvalensis, Proteus mirabilis, P. vulgaris, Rhodococcus equi, Salmonella including S. arizonae, S. cholerasuis, S. enteritidis, S. gallinarum-pullorum, S. meleagridis, S. paratyphi, A, B, C, S. typhi, S. typhimurium, Shigella including S. boydii, S. dysenteriae, type 1, S. flexneri, S. sonnei, Sphaerophorus necrophorus, Staphylococcus aureus, Streptobacillus moniliformis, Streptococcus including S. pneumoniae, S. pyogenes, Treponema pallidum, T. carateum, Vibrio cholerae, V. parahemolyticus, V. vulnificus, Yersinia enterocolitica, Bartonella, Brucella including B. abortus, B. canis, B. suis, B. melitensis, Burkholderia (Pseudomonas) mallei, B. pseudomallei, Coxiella burnetii, Francisella tularensis, Mycobacterium bovis (except BCG strain, BSL II—Bacterial Agents Including Chlamydia), M. tuberculosis, Mycobacteria other than tuberculosis (MOTT), Pasteurella multocida type B—“buffalo” and other virulent strains. Rickettsia akari, R. australis, R. canada, R. conorii, R. prowazekii, R. rickettsii, R, siberica, R. tsutsugamushi, R. typhi (R. mooseri), Yersinia pestis.

The antigen may be derived from a viral pathogen. For example, in some embodiments, the antigen is derived from a viral pathogen selected from among Adenoviruses, human—all types, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Calciviruses, Coronaviruses. Flaviviruses (Togaviruses)—Group B Arboviruses, Dengue virus serotypes 1, 2, 3, and 4, Yellow fever virus vaccine strain 17D, Hepatitis A, B, C, D, and E viruses, the Cytomegalovirus, Epstein Barr virus, Herpes simplex types 1 and 2, Herpes zoster, Human herpesvirus types 6 and 7, Influenza viruses types A, B, and C, Papovaviruses, Papilloma viruses, Newcastle disease virus, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, and Orbivirus (Colorado tick fever virus), Rabies virus, Vesicular stomatitis virus, Rubivirus (rubella), Semliki Forest virus, St. Louis encephalitis virus, Venezuelan equine encephalitis virus, Venezuelan equine encephalomyelitis virus, Arenaviruses (a.k.a. South American Haemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2, Simian immunodeficiency virus (SIV), Vesicular stomatitis virus, Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, and Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, Variola major virus (Smallpox virus), Variola minor virus (Alastrim), African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Japanese encephalitis virus, Lumpy skin disease virus, Malignant catarrhal fever virus, Menangle virus, Newcastle disease virus (VVND), Peste Des Petits Ruminants virus, Rinderpest virus, Sheep pox virus, Swine vesicular disease virus, Vesicular stomatitis virus (exotic).

The antigen may be derived from a parasite. For example, in some embodiments, the antigen is derived from a parasite selected from among Ancylostoma human hookworms including A. duodenale, A. ceylanicum, Ascaris including Ascaris lumbricoides suum, Babesia including B. divergens, B. microti, Brugia filaria worms including B. malayi, B. timori, Coccidia, Cryptosporidium including C. parvum, Cysticercus cellulosae (hydatid cyst, larva of T. solium), Echinococcus including E. granulosis, E. multilocularis, E. vogeli, Entamoeba histolytica, Enterobius, Fasciola including F. gigantica, F. hepatica, Giardia including G. lamblia, Heterophyes, Hymenolepis including H. diminuta, H. nana, Isospora, Leishmania including L. braziliensis, L. donovani, L. ethiopia, L. major, L. mexicana, L. peruvania, L. tropica, Loa loa filaria worms, Microsporidium, Naegleria fowleri, Necator human hookworms including N. americanus, Onchocerca filaria worms including, O. volvulus, Plasmodium cynomologi, P. falciparum, P. malariae, P. ovale, P. vivax, Sarcocystis including S. sui hominis, Schistosoma including S. haematobium, S. intercalatum, S. japonicum, S. mansoni, S. mekongi, Strongyloides including S. stercoralis, Taenia solium, Toxocara including T. canis, Toxoplasma including T. gondii, Trichinella spiralis, Trypanosoma including T. brucei brucei, T. brucei gambiense, T. brucei rhodesiense, T. cruzi, or Wuchereria bancrofti filaria worms.

The antigen may be a fungal pathogen. For example, in some embodiments, the antigen is derived from a fungal pathogen selected from among Aspergillus fumigates, Blastomyces dermatitidis, Cladosporium bantianum, Candida albicans, C. (Xylohypha) trichoides, Cryptococcus neoformans, Dactylaria galopava (Ochroconis gallopavum), Epidermophyton, Exophiala (Wangiella) dermatitidis, Fonsecaea pedrosoi, Microsporum, Paracoccidioides braziliensis, Penicillium marneffei, Pneumocystis carinii, Sporothrix schenckii, Trichophyton, Coccidioides immitis, Coccidioides posadasii, Histoplasma capsulatum, H. capsulatum var. duboisii.

The antigen may be a toxin. In some embodiments, the antigen is a toxin selected from among Abrin, Botulinum neurotoxins, Clostridium perfringens epsilon toxin, Conotoxins, Diacetoxyscirpenol, Ricin, Saxitoxin, Shiga-like ribosome inactivating proteins, Shigatoxin, Staphylococcal enterotoxins, T-2 toxin, Tetrodotoxin.

In some embodiments, the antigen is selected from among Hepatitis B surface antigen (HBsAg), B. burgdorferi OspA, HPV L1, RSV F protein, Influenza hamagglutanin, Influenza stem-loop region, Influenza M2, P. falciparum merozoite surface protein 1-10, GLURP, SERA, S-antigen, 6-cys family, AMA1, EBA175, 140, 181, MTRAP, PTRAMP, ASP, Rh1, 2a, 2b, 4, 5, RAP1, 2, 3, RAMA, RHOPH1, 2, 3, P. vivax circumsporozoite protein, sporozoite surface proetin2, SSP2/TRAP, CSP-N, CSP-R, CSP-C, MSP-1, MSP-9, DBPRIII, AMA-1, Pvs25, Pvs28, S. aureus capsular polysaccharide, poly-N-acetyl glucosamine, HIV gp120, gp41, and Dengue virus conserved regions.

C. Epitopes of Allergens

The epitope used to sensitize or tolerize the subject can be an epitope of an allergen. Subjects may be sensitized or tolerized to an allergen before, during, or after the subject is exposed to the antigen. Allergens can be naturally occurring, or artificial such as allergens contained in allergy vaccines. Examples of allergens include, but are not limited to, animal products (for example, Fel d 1, fur dander, cockroach calyx, wool, dust mite excretion), drugs (for example, penicillin, sulfonamides, salicylates, local anaesthetic), food (for example, celery and celeriac, corn, eggs (e.g., albumin), fruit, legumes (for example, beans, peas, peanuts, soybeans), milk, seafood (e.g., shellfish), sesame, soy, tree nuts (for example, pecans, almonds), wheat, insect venom (for example, fire ants, bee sting venom, wasp sting venom), latex, metal, plant pollen (for example, grass (e.g., ryegrass, timothy-grass, weeds (e.g., ragweed, plantago, nettle, Artemisia vulgaris, chenopodium album, sorrel), and trees (e.g., birch, alder, hazel, hornbeam, aesculus, willow, poplar, platanus, tilia, olea, Ashe juniper)).

In some embodiments, the allergen is a latex protein, for example, unprocessed latex sap, raw latex containing ammonia, or finished latex product in which the proteins have been exposed to chemicals and high temperatures.

In some embodiments, the allergen is the allergen of a mite, for example, Dermatophagoides farinae, Dermatophagoides pteronyssinus, Acarus siro, Blomia tropicalis, Chortoglyphus arcuatas, Euroglyphus maynei, Lepidoglyphus destructor, Tyrophagus putrescentiae, or Glyphagus demesticus.

In some embodiments, the allergen is from venom, for example, Bombus spp., Vespa crabro, Apis mellifera, Dolichovespula spp., Polistes spp., Vespula spp., Dolichovespula maculata, or Dolichovespula arenaria.

In some embodiments, the allergen is from an insect, for example, Camponotus pennsylvanicus, Solenopsis invicta, Solenopsis richteri, Periplaneta americana, Blattella germanica, Blatta orientails, Tebanus spp., Musca domestica, Ephemeroptera spp., Culicidae sp., or Heterocera spp.

In some embodiments, the allergen is epithelia, dander, or hair from an organism, for example, Serinus canaria, Felis catus (domesticus), Bos taurus, Gallus gallus (domesticus), Canis familiaris, Arias platyrhynchos, Meriones unguiculatus, Capra hircus, Anser domesticus, Cavia porcellus (cobaya), Mesocrietus auratus, Sus scrofa, Equus caballus, Mus musculus, Psittacidae, Columba fasciata, Oryctolagus cuniculus, Rattus norvegicus, or Ovis aries.

In some embodiments, the allergen is from fungi, for example, Cephalosporium acremonium, Alternaria tenuis, Aspergillus glaucus, Aspergillus flavus, Aspergillus fumigatus, Aspergillus nidulans, Aspergillus niger, Aspergillus terreus, Aspergillus versicolor, Aureobasidium pullulan (Pullularia pullulans), Drechslera sorokiniana, Helminthosporium sativum, Botrytis cinerea, Candida albicans, Chaetomium globosum, Cladosporium herbarum, Cladosporium sphaerospennum (Homodendrum hordei), Drechslera spicifera (Curvularia spicifera), Epicoccum nigrum (Epicoccum purpurascens), Epidermophyton floccosum, Fusarium moniliforme, Fusarium solani, Geotrichum candidum, Gliocladium viride, Helminthosporium solani, Microsporum canis, Mucor circinelloidesf circinelloides, Mucor circinelloidesf lusitanicus, Mucor plumbous, Mycogone perniciosa, Neurospora intermedia, Nigrospora oryzae, Paecilomyces variotii, Penicillum brevicompactum, Penicillum camembertii, Penicillum chrysogenum, Penicillum digitatum, Penicillum expansum, Penicillum notatum, Penicillum roquefortii, Phoma betae, Phoma herbarum, Rhizopus oryzae, Rhizopus stolonifer, Rhodotorula mucilaginosa, Saccharomyces cerevisiae, Scopulariopsis brevicaulis, Serpula lacrymans, Setosphaeria rostrata, Stemphylium botryosum, Stemphylium solani, Trichoderma harzianum, Trichophyton mentagrophytes, Trichophyton rubrum, or Trichothecium roseum.

In some embodiments, the allergen is from a smut, for example, Ustilago nuda, Ustilago cynodontis, Ustilago maydis, Sporisorium cruentum, Ustilago avenae, or Ustilago tritici.

In some embodiments, the allergen is from a grass, for example, Paspalum notatum, Cynodon dactylon, Poa compressa, Bromus inennis, Phalaris arundinacea, Zea mays, Elytrigia repens (Agropyron repens), Sorghum haelpense, Poa pratensis, Festuca pratensis (elatior), Avena sativa, Dactylis glomerata, Agrostis gigantea (alba), Secale cereale, Leymus (Elymus) condensatus, Lolium perenne ssp. multiflorum, Lolium perenne, Anthoxanthum odoratum, Phleum pratense, Holcus lanatus, Triticum aestivum, or Elymus (Agropyron) smithii.

In some embodiments, the allergen is from a weed, for example, Atriplex polycarpa, Baccharis halimifolia, Baccharis sarothroides, Hymenoclea salsola, Amaranthus hybridus, Xanthium strumarium (commune), Rumex crispus, Eupathium capillifolium, Solidago spp., Amaranthus tuberculatus (Acnida tamariscina), Allenrolfea occidentalis, Chenopodium botrys, Kochia scoparia, Chenopodium album, Iva xanthifolia, Iva angustifolia, Chenopodium ambrosioides, Artemisia vulgaris, Artemisia ludoviciana, Urtica dioica, Amaranthus spinosus, Plantago lanceolata, Iva axillaris, Atriplex lentiformis, Ambrosia dumosa, Ambrosia acanthicarpa, Ambrosia trifida, Ambrosia artemisiifolia, Ambrosia confertiflora, Ambrosia bidentata, Ambrosia psilostachya, Salsola kali (pestifer), Artemisia californica, Artemisiafrigida, Artemisia tridentata, Atriplex wrightii, Atriplex confertifolia, or Artemisia annua.

In some embodiments, the allergen is from a tree, for example, Acasia spp., Alnus glutinosa, Alnus rubra, Alnus incana ssp. rugosa, Alnus rhombifolia, Fraxinus velutina, Fraxinus pennsylvanica, Fraxinus latifolia, Fraxinus americana, Populus tremuloides, Myrica cerifera, Fagus grandifolia (americana), Casuarina equisetifolia, Betula lenta, Betula pendula, Betula nigra, Betula occudentalis (fontinalis), Betula populifolia, Acer negundo, Cryptomeria japonica, Juniperus ashei (sabinoides), Juniperus virginiana, Tamarix gallica, Populus balsamifera ssp. trichocarpa, Populus deltoides, Populusfremontii, Populus wislizeni, Populus monilifera (sargentii), Cupressus arizonoca, Taxodium distichum, Cupressus sempervirens, Ulmus americana, Ulmus crassifolia, Ulmus pumila, Eucalyptus globulus, Celtis occidentalis, Corylus americana, Corylus avellana, Carya ovata, Carya laciniosa, Carya alba, Juniferus monosperma, Juniperus princhotii, Juniperus scopulorum, Juniperus occidentalis, Robinia pseudoacacia, Mangifera indica, Acer macrophyllum, Acer rubrum, Acer saccharum, Melaleuca quinquenervia (leucadendron), Prosopis glandulosa (juliflora), Broussonetia papyrifera, Morus rubra, Morums alba, Quercus gambelii, Quercus velutina, Quercus macrocarpa, Quercus kelloggii, Quercus agrifolia, Quercus lobata, Quercus ilex, Quercus stellata, Quercus rubra, Quercus dumosa, Quercus virginiana, Quercus nigra, Quercus garryana, Quercus alba, Olea europaea, Elaegnus angustifolia, Citrus sinensis, Arecastrum romanzoffianum (Cocos plumosa), Carya illnoensis, Schinus molle, Schinus terebinthifolius, Pinus taeda, Pinus strobus, Pinus palustris, Pinus ponderosa, Pinus elliottii, Pinus virginiana, Pinus monticola, Pinus echinata, Populus nigra, Populus alba, Ligustrum vulgare, Liquidambar styraciflua, Platanus occidentalis, Platanus orientalis, Platanus racemosa, Platanus acerifolia, Juglans nigra, Juglans californica, Juglans regia, Salix lasiolepsis, Salix nigra, or Salix discolor.

In some embodiments, the allergen is from a flower, for example, Chrysanthemum leucanthemum, Taraxacum officinale, or Helianthus annuus.

In some embodiments, the allergen is from a farm plant, for example, Medicago sativa, Ricinus communis, Trifolium pratense, Brassica spp., or Beta vulgaris.

In some embodiments, the allergen is from plant food (an edible plant), for example, Prunus dulcis, Malus pumila, Prunus armeniaca, Musa paradisiaca (sapientum), Hordeum vulgare, Phaseolus lanatus, Phaseolus vulgaris, Phaseolus sp., Phaseolus sp., Phaseolus vulgaris, Rubus allegheniensis, Vaccinium sp., Brassica oleracea var. botrytis, Fagopyrum esculentum, Brassica oleracea var. capitata, Theobroma cacao, Cucumis melo, Daucus carota, Brassica oleracea var. botrytis, Apium graveolens var. dulce, Prunus sp., Cinnamomum verum, Coffea arabic, Zea mays, Vaccinium macrocarpon, Cucumis sativus, Allium sativum, Zingiber officinale, Vitis sp., Citrus paradisi, Humulus lupulus, Citrus limon, Lactuca sativa, Agaricus campestris, Brassica sp., Myristica fragrans, Avena sativa, Olea europaea, Allium cepa var. cepa, Citrus sinensis, Vigna unguiculata, Pisum sativum, Prunus persica, Pyrus communis, Piper nigrum, Capsicum annuum var. annuum, Ananas comosus, Ipomoea batatas, Solanum tuberosum, Rubus idaeus var. idaeus, Oryza sativa, Secale cereale, Sesamum orientale (indicum), Glycine max, Spinacia oleracea, Cucurbita pepo var. melopepo, Fragaria chiloensis, Lycopersicon esculentum (lycopersicum), Brassica rapa var. rapa, Vanilla planifolia, Citrullus lanatus var. lanatus, or Triticun aestivum.

In some embodiments the allergen is from fish or shellfish, for example, Micropterus sp., Ictalurus punctatus, Mercenaria mercenaria, Gadus morhua, Callinectes sapidus, Platichthys sp., Hippoglossus sp., Homarus americanus, Scomber scombrus, Crassostrea virginica, Sebastes marinus, Salmo salar, Clupeiformes, Pecten magellanicus, Penaeus sp., Salvelinus sp., or Thunnus sp.

In some embodiments, the allergen is an animal food product, for example, from Bos taurus, Ovis aries, or Sus scrofa.

In some embodiments, the allergen is a poultry product, for example, chicken (Gallus gallus) products or turkey (Meleagris gallopavo) products.

In some embodiments, the allergen is from a dairy product, for example, bovine casein or bovine milk.

In some embodiments, the allergen is a nut, for example, Bertholletia excelsa, Anacardium oceidentale, Cocos nucifera, Corylus americana, Arachis hypogaea, Carya illinoensis, Juglans nigra, or Juglans regia.

In some embodiments, the allergen is dust, for example, barley grain dust, corn grain dust, house dust, mattress dust, oat grain dust, wheat grain dust, upholstery dust, or latex dust.

D. Epitopes of Autoantigens

The epitope used to sensitize or tolerize the subject can be an epitope of an autoantigen. In some embodiments, the antigen is an autoantigen associated with an autoimmune disorder. In some embodiments, the autoimmune disorder is a cell or organ-specific autoimmune disorder, and the autoantigen is selected from among: acetylcholine receptor (myasthenia gravis), actin (chronic active hepatitis, primary biliary cirrhosis), adenine nucleotide translocator (ANT) (dilated cardiomyoapthy, myocarditis), beta-adrenoreceptor (dilated ° cardiomyopathy), aromatic L-amino acid decarboxylase (autoimmune polyendocrine syndrome type I (APS-1)), asialoglycoprotein receptor (autoimmune hepatitis), bactericidal/permeability-increasing protein (Bpi) (cystic fibrosis vasculitides), calcium-sensing receptor (acquired hypoparathyroidism), cholesterol side-chain cleavage enzyme (CYPIIa) (APS-1), collagen type IV alpha3-chain (Goodpasture syndrome), cytochrome P450 2D6 (CYP2D6) (autoimmune hepatitis), desmin (Crohn disease, coronary artery disease), desmoglein 1 (pemphigus foliaceus), desmoglein 3 (pemphigus vulgaris), F-actin (autoimmune hepatitis), GM ganglioside (Guillain-Barre syndrome), glutamate decarboxylase (GAD65) (type 1 diabetes, stiff man syndrome), glutamate receptor (GLUR) (Rasmussen encephalitis), H/K ATPase (autoimmune gastritis), 17-alpha-hydroxylase (CYP17) (APS-1), 21-hydroxylase (CYP21) (Addison disease), IA-2 (ICA512) (type 1 diabetes), insulin (type 1 diabetes, insulin hypoglycemic syndrome (Hirata disease), type B insulin resistance, acanthosis, systemic lupus erythematosus (SLE)), intrinsic factor type 1 (pernicious anemia), leukocyte function-associated antigen (LFA-1) (treatment-resistant lyme arthritis), myelin-associated glycoprotein (MAG) (polyneuropathy), myelin basic protein (multiple sclerosis, demyelinating disease), myelin oligodendrocyte glycoprotein (MOG) (multiple sclerosis), myosin (rheumatic fever), p-80-Coilin (atopic dermatitis), pyruvae dehydrogenase complex-E2 (PDC E2) (primary biliary cirrhosis), sodium iodide symporter (NIS) (Graves disease, autoimmune hypothyroidism), SOX-10 (vitiligo), thyroid and eye muscle shared protein (autoimmune thyroiditis), thyroid peroxidase (autoimmune Hashimoto thyroiditis), thyrotropin receptor (Graves disease), tissue transglutaminase (celiac disease), transcription coactivator p75 (atopic dermatitis), tryptophan hydroxylase (APS-1), tyroisinase (vitiligo, metastatic melanoma), and tyrosine hydroxylase (APS-1), wherein the associated autoimmune disorder(s) is listed parenthetically immediately after each autoantigen.

In some embodiments, the autoimmune disorder is a systemic autoimmune disorder, and the autoantigen is selected from among: ACTH (ACTH deficiency), aminoacyl-tRNA histidyl synthetase (myositis, dermatomyositis), aminoacyl-tRNA synthetase (polymyositis, dermatomyositis), cardiolipin (SLE), carbonic anhydrase II (SLE, Sjogren syndrome, systemic sclerosis), collagen (rheumatoid arthritis (RA), SLE, progressive systemic sclerosis), centromere-associated protein (systemic sclerosis), DNA-dependent nucleosome-stimulated ATPase (dermatomyositis), fibrillarin (scleroderma), fibronectin (SLE, RA, morphea), glucose-6-phosphate isomerase (RA), Beta2-glycoprotein I (Beta2-GPI) (primary antiphospholipid syndrome), golgin (95, 97, 160, and/or 180) (Sjogren syndrome, SLE, RA), heat shock protein (various immune related disorders), hemidesmosomal protein 180 (bullous pemphigoid, herpes gestationis, cicatricial pemphigoid, histone H2A-H2B-DNA (SLE), IgE receptor (chronic idiopathic urticaria), keratin (RA), Ku-DNA-protein kinase (SLE), Ku-nucleoprotein (connective tissue syndromes), La phosphoprotein (La 55-B) (Sjoren syndrome), myeloperoxidase (necrotizing and cescentic glomerulonephritis (NCGN), system vasculitis), proteinase 3 (PR3) (Wegener granulomatosis, Churg-Strauss syndrome), RNA polymerase I-III (RNP) (systemic sclerosis, SLE), signal recognition protein (SRP54) (polymyositis), topoisomerase-1 (Scl-70) (scleroderma, Raynaud syndrome), tubulin (chronic liver disease, visceral leishmaniasis), and vimentin (systemic autoimmune disease), wherein the associated autoimmune disorder(s) is listed parenthetically immediately after each autoantigen.

In some embodiments, the autoimmune disorder is a plasma protein autoimmune disorder or cytokine autoimmune disorder, and the autoantigen is selected from among: C1 inhibitor (autoimmune C1 deficiency), C1q (SLE, membrane proliferative glomerulonephritis (MPGN)), cytokine (e.g., IL-1 alpha, IL-1beta, IL-, IL-10, LIF) (RA, systemic sclerosis), factor II (prolonged coagulation time), factor V (prolonged coagulation time), factor VII (prolonged coagulation time), factor VIII (prolonged coagulation time), factor IX (prolonged coagulation time), factor X (prolonged coagulation time), factor XI (prolonged coagulation time), factor XII (prolonged coagulation time), thrombin (prolonged coagulation time), vWF (prolonged coagulation time), glycoprotein IIb/IIIg and Ib/IX (autoimmune thrombocytopenia purpura), IgA (immunodeficiency), and oxidized LDL (OxLDL) (atherosclerosis), wherein the associated autoimmune disorder(s) is listed parenthetically immediately after each autoantigen.

In some embodiments, the autoimmune disorder is a cancer or paraneoplastic autoimmune disorder, and the autoantigen is selected from among: amphiphysin (neuropathy, small lung cell cancer), cyclin B 1 (hepatocellular carcinoma), DNA topoisomerase II (liver cancer), desmoplakin (paraneoplastic pemphigus), gephyrin (paraneoplastic stiff man syndrome), Hu protein (paraneoplastic encephalomyelitis), neuronal nicotinic acetylcholine receptor (subacute autonomic neuropathy, cancer), p53 (cancer, SLE), p62 (IGF-II mRNA-binding protein) (hepatocellular carcinoma), recoverin (cancer-associated retinopathy), R1 protein (paraneoplastic opsoclonus myoclonus ataxia), beta IV spectrin (lower motor neuron syndrome), synaptotagmin (Lambert-Eaton myasthenic syndrome), voltage-gated calcium channels (Lambert-Eaton myasthenic syndrome) and Yo protein (paraneoplastic cerebellar degeneration).

In some embodiments, the antigen is an endogenous antigen that is an aberrantly expressed polypeptide. Examples of such endogenous antigens include, but are not limited to, amyloid beta (A-beta or Aβ), alpha synuclein, cystatin C, tau, ABri, ADan, superoxide dismutase (SOD), mutant Huntington, PrP^{sc} or a fragment of any of the foregoing.

A-beta is toxic to neurons, and its accumulation as plaques in the brains of Alzheimer's disease (AD) patients is thought to contribute to the neurodegeneration that is characteristic of the disorder. A-beta protein is generated when the amyloid precursor protein is cleaved by enzymes. Different types of A-beta can be produced enzymatically, with A-beta 40 and A-beta 42 cleavage products being predominant and prone to aggregate into plaques. Immunization with A-beta and A-beta derivatives has been shown to reduce amyloid burden and improve cognition in AD model mice (Sigurdsson E. M. et al., “An attenuated immune response is sufficient to enhance cognition in an Alzheimer's disease mouse model”, The Journal of Neuroscience, 24(28):6277-6282 (2004)). The A-beta peptide has become a major therapeutic target in AD. Active and passive A-beta immunotherapies have been shown to lower cerebral A-beta levels and improve cognition in animal models of AD. In a phase II clinical trial, administration of an A-beta vaccine to humans was stopped when ˜6% of the immunized patients developed meningoencephalitis; however, some plaque clearance and clinical improvements were observed in patients following immunization (Lemere C. A. et al., “Can Alzheimer Disease be prevented by amyloid-β immunotherapy”, Nature Reviews Neurology, 6(2):108-119 (2010)). Based upon preclinical studies and the limited human data available, A-beta immunotherapy might be most effective in preventing or slowing the progression of AD when patients are immunized before or in the early stages of disease onset (Lemere C. A. et al., 2010). Furthermore, biomarkers for AD and imaging modalities may be used to identify pre-symptomatic, at-risk individuals who might benefit from immunization.

An epitope of A-beta (for example, A-beta 40 or A-beta 42) or an A-beta derivative may be co-administered with an Fc region in accordance with the present invention. For example, the A-beta derivative may be one engineered to elicit a modified immune response (Wang C. Y. et al., “Site-specific UBITh amyoid-beta vaccine for immunotherapy of Alzheimer's disease”, Vaccine, 25(16):3041-3052 (2007)). Ideally, the elicited immune response will include functional immunogenicities to neutralize the toxic activity of A-beta and either prevent plaque deposition or promote clearance of plaques.

Many studies have failed to detect the presence of immune responses to infectious prions during the course of prion disease (transmissible spongiform encephalopathies, which include Crutzfeldt-Jacob disease in humans and bovine spongiform encephalopathy and scrapie in animals). The pathogenesis of prion diseases involves the transformation of the mainly alpha-helical normal cellular prion protein, PrP^{C}, into a disease-associated isoform, PrP^{Sc}, that acquires increased beta-sheet content. detergen insolubility and resistance to proteases (Tayebi M. et al., “Immunisation with a synthetic prion protein-derived peptide prolongs survival times of mice orally exposed to the scrapie agent”, Journal of General Virology, 90:777-782 (2009); Rubeinstein R. et al., “Immune surveillance and antigen conformation determines humoral immune response to the prion protein immunogen”, Journal of Neuro Virology, 5:401-413 (1999); Schwarz A. et al., Neuroscience Letters, 350(3):187-189 (2003)). Thus, the epitope used in the compositions and methods of the invention may be an epitope of a human or animal prion disease-associated antigen (for example, PrP^{Sc}), a synthetic prion protein-derived peptide (for example, PrP105-125), or a PrP fragment (for example, PrP90-230). For example, by co-administering an epitope associated with a prion disease with an IgM Fc region, the subject's immune system can be stimulated to recognize and attack the prion disease-associated antigen. This immunotherapeuetic approach can provide a therapy and prophylaxis against prion disease.

E. Miscellaneous Epitopes

In some embodiments of the invention, the epitope used in tolerizing the subject is the epitope of an implant to be introduced into the subject. The method may further comprise introducing the epitope- or antigen-bearing implant into the subject after tolerization to the epitope. Such implants may include, for example, electrically powered implants (for example, artificial pacemakers), bioimplants (biomaterial surgically implanted in a subject's body to replace damaged tissue (for example, orthopedic reconstructive prosthesis), cardiac prostheses (artificial valves), skin, and cornea), contraceptive implants, dental implants, orthopedic implants, and adhesion prevention devices. Examples of implant materials that may bear epitopes include latex; silicone; metals, such as cobalt chrome (Co—Cr) alloys, titanium, and titanium alloys; polymers, such as ultra-high molecular weight polyethylene (UHMWPE) and polymethyl methacrylate cement (PMMA); and bioceramics, such as hydroxyapatite and Bioglass.

### IV. COMBINATION AND ADJUNCTIVE THERAPIES

In addition to the epitope(s) and Fc region, the methods and compositions of the invention may incorporate other immunomodulatory or non-immunomodulatory agents.

Under some circumstances, it may be desirable to sensitize a subject to an epitope and subsequently tolerize the subject to reduce unwanted immune response from the sensitization. For example, in some embodiments, the subject has cancer, the antigen is a cancer antigen identified in the subject, the cancer is eliminated or attenuated after sensitizing the subject to the epitope, and the method further comprises tolerizing the subject to the epitope after the cancer is eliminated or attenuated in order to reduce unwanted autoimmune reaction from the sensitization.

Endogenous mechanisms for controlling autoimmune responses (natural tolerance) and of inducing tolerance (adaptive tolerance) exist. T-regulatory lymphocytes (T-regulatory cells or T-regs) are a specialized subset of CD4⁺ T cells implicated in the suppression of immune response, fulfilling an important role in the maintenance of immune homeostasis (De Groot A. S. et al., “Activation of natural regulatory T cells y IgG Fc derived peptide “Tregitopes”, Blood, 112(8):3303-3311 (2008)). T-regs differ from other CD4⁺ cells in expressing high levels of CD25 and by expression of the forkhead/winged helix transcription factor (Foxp3). Under some circumstances, it may be desirable to inhibit Treg activity and/or reduce the number of T-regs in a subject (i.e., to inhibit the immunosuppressive effects of T-regs) prior to sensitizing a subject to an epitope. Accordingly, in some embodiments of the sensitization method, the subject has reduced T-regulatory cell activity and/or reduced numbers of T-regulatory cells at the time of co-administration of the epitope and the IgM Fc region. Reduced T-regulatory cell activity and/or reduced T-regulatory cell numbers may be achieved in a subject by administering an inhibitor of T-regulatory cells to the subject. The reduced T-regulatory cell activity and/or reduced numbers of T-regulatory cells can be relative to the normal activity and/or cell numbers in the subject and/or relative to a normal control population, for example.

Agents capable of inhibiting T-reg immunosuppressive activity and/or T-reg numbers, and which may be utilized in the invention, are known (Cohen A. D. et al., “Agonist anti-GITR antibody enhances vaccine-induced CD8(+) T-cell responses and tumor immunity”, Cancer Res 66:4904-49-12 (2006); Onizuka S. et al., “Tumor rejection by in vivo administration of anti-CD25 (interleukin-2 receptor alpha) monoclonal antibody” Cancer Res, 59:3128-3133 (1999); Shimizu J. et al., “Induction of tumor immunity by removing CD25+CD4+ T cells: a common basis between tumor immunity and autoimmunity,” J. Immunol., 163:5211-5218 (1999); Tanaka H. et al., “Depletion of CD4+CD25+ regulatory cells augments the generation of specific immune T cells in tumor-draining lymph nodes,” J. Immunother., 25:207-217 (2002); Ko K. et al., “Treatment of advanced tumors with agonistic anti-GITR mAB and its effects on tumor-infiltrating Foxp3+CD25+CD4+ regulatory T cells,” J. Exp. Med., 202:885-891 (2005); Ghiringhelli F. et al., “CD4+CD25+ regulatory T cells suppress tumor immunity but are sensitive to cyclophosphamide which allows immunotherapy of established tumors to be curative,” Eur. J. Immunol., 34:336-344 (2004); Galustian C. et al., “The anti-cancer agents lenalidomide and pomalidomide inhibit proliferation and function of T regulatory cells” Cancer Immunol Immunother., 58(7):1033-1045 (2009); Houot R. et al., “T-cell modulation combined with intratumoral CpG cures lymphoma in a mouse model without the need for chemotherapy”, Blood, 113(15):3546-3552 (2009); Nizar S. et al., “T-regulatory cell modulation: the future of cancer immunotherapy?”, British Journal of Cancer, 100:1697-1703; and Dias de Rezende, L. C. et al., “Regulatory T cell as a target for cancer therapy”, Arch. Immunol. Ther. Exp., 58:179-190 (2010)).

Examples of T-reg inhibitors include, but are not limited to, lenalidomide, pomalidomide, oxazaphosphorines such as cyclophosphamide, anti-CD25 monoclonal antibody, IL-2Ra monoclonal antibody, and anti-glucocorticoid-induced tumor necrosis factor receptor (anti-GITR) monoclonal antibody. In some embodiments, the inhibitor of T-regulatory cells reduces the activity and/or reduces the number of CD4⁺CD25_{Hi}FoxP3⁺ natural T-regulatory cells in the subject. In some embodiments, the sensitization method of the invention comprises administering a T-regulatory cell inhibitor to the subject, and subsequently administering the epitope and the IgM Fc region to the subject.

In some embodiments, the subject has a B-cell malignancy, and the sensitization method comprises sensitizing a subject to an epitope by administering a T-reg inhibitor to the subject (such that T-reg immunosuppressive activity and/or T-reg numbers are reduced in the subject) and subsequently administering an idiotype vaccine comprising the B-cell malignancy's idiotype, and administering the IgM Fc region to the subject.

Another aspect of the invention features a method for directing an immune response in a subject, comprising determining the T-regulatory (T-reg) cell level (T-reg cell number and/or T-reg activity) in the subject; wherein if the T-reg cell level is consistent with a normal T-reg cell level, an effective amount of a T-reg cell inhibitor is administered to the subject prior to administration of a composition of the invention (a sensitizing composition). The T-reg cell level can be determined by obtaining one or more biological samples from the subject (for example, blood, peripheral blood, synovial fluid, or other biological tissue or fluid that may be sampled and in which T-reg cells are found) and determining the T-reg cell level in the sample(s) prior to administration of a composition of the invention. Ideally, the immunosuppressive effect of T-reg cells in the subject is inhibited or reduced to maximize the clinical effectiveness of the subsequently administered composition. Thus, preferably, the T-reg cell inhibitor is administered to the subject until the T-reg cell level in the subject is below that of a threshold, immunosuppressive T-reg cell level. In some embodiments, the T-reg cell level is determined two or more times and the T-reg cell inhibitor is administered to the subject until the T-reg cell level in the subject is below that of a threshold, immunosuppressive T-reg cell level, prior to administration of the composition. T-reg cell level can be determined by methods known in the art. For example, T-reg cells in a sample can be quantified by flow cytometry. Sub-populations of T-reg cells can be targeted for level determination, such as CD4+ CD25HIFoxp3+ cells.

In methods of the invention, determining T-reg cell level in a subject may involve comparing the observed level to that of a reference T-reg cell level or suitable control (for example, to assess whether T-reg cell level is below, equal to, or above a threshold level, e.g., a “normal” level). A “suitable control” is a predetermined value associated with T-reg cell level useful for comparison purposes, which can take many different forms. Exemplary forms include, but are not limited to, for example, T-reg cell numbers, a transcription rate, mRNA level, translation rate, protein level, protein structure, biological activity, cellular characteristic or property, genotype, phenotype, enzymatic activity etc. associated with T-reg cells. In some embodiments, a “suitable control” is a predetermined T-reg cell activity, which is compared to T-reg cell activity in a sample obtained from a subject being identified as suitable or not suitable for treatment with a composition of the invention. In other embodiments, a “suitable control” is a predetermined T-reg cell number, which is compared to T-reg cell number in a sample obtained from a subject being identified as suitable or not suitable for treatment with a composition of the invention. In other embodiments, a “suitable control” is a predetermined T-reg cell number and activity, which is compared to T-reg cell number and activity in a sample obtained from a subject being identified as suitable or not suitable for treatment with a composition of the invention. In other embodiments, a “suitable control” is a predetermined T-reg cell level, which is compared to a T-reg cell level in a sample obtained from a subject in which a clinical measure was achieved, for example an T-reg cell level obtained from cells in a subject who reached or failed to reach a desired immune response.

In some embodiments, a “suitable control” can be a single cut-off value, such as a median or mean. A single cut-off value can be established, for example, based upon comparative groups, such as in groups having a T-reg level which reduces a desirable immune response to a composition of the invention and/or which interferes or impedes a desired clinical outcome following treatment with a composition of the invention. For example, samples can be derived from various individuals or blood banks and a T-reg cell level can be measured in each sample prior to being subjected to treatment with a composition of the invention. Consequently, a single cut-off value can be based on the mean of T-reg cell number and/or activity in samples which are immunosuppressive to an extent that reduces a desirable immune response to a composition of the invention and/or which interferes or impedes a desired clinical outcome following treatment with a composition of the invention. Another comparative group can be, for example, a T-reg cell level in a group of individuals with a family history of successful treatment with a composition of the invention and a group without such a family history. Another comparative group can be, for example, a T-reg cell level in a group of individuals with a history of treatment with a composition of the invention having achieved maximal immune response and/or clinical outcome and a group having not achieved maximal immune response and/or clinical outcome.

In some embodiments of the methods of the present invention, a subject is identified as being suitable for treatment with a composition of the invention (e.g., a sensitizing composition) if the T-reg cell level measured in a sample (for example, blood sample) obtained from the subject is consistent with an “suitable control.” By “consistent with a suitable control,” is meant that the T-reg cell level is either equal to or below a predetermined T-reg cell level control, in case of a single cut-off value, or the T-reg cell level falls within a range for a predetermined T-reg cell level control. In some embodiments, a subject is identified as being suitable for treatment with a composition of the invention if the T-reg cell level in a sample from the subject is consistent with a maximal immune response (non-immunosuppressed). By “consistent with a maximal immune response,” is meant that the T-reg cell level is either equal to or lower than a predetermined “immunosuppressive level,” in case of a single cut-off value, or the T-reg cell level falls within a range for a predetermined immunsuppressive level. In this way, it can be determined whether a subject is suitable for treatment with a composition of the invention (e.g., the T-reg cell level in a sample from the subject is consistent with a maximal immune response or “non-immune suppressed) or whether the subject should be administered a T-reg cell inhibitor (e.g., the T-reg cell level in a sample from the subject is inconsistent with or below a maximal immune response or “immune suppressed”).

### V. B-CELL MALIGNANCIES

Exemplary disorders which may be treated using the methods of the invention include (but are not limited to) B-cell malignancies and in particular, B-cell derived cancers or neoplasms such as, for example, non-Hodgkin's lymphoma, Hodgkin's lymphoma, chronic lymphocytic leukemia, mantle cell lymphoma and multiple myeloma. Additional B-cell derived cancers include, for example, B-cell prolymphocytic leukemia, lymphoplasmocytic leukemia, splenic marginal zone lymphoma, marginal zone lymphoma (extra-nodal and nodal), plasma cell neoplasms (e.g., plasma cell myeloma, plasmacytoma, monoclonal immunoglobulin deposition diseases, heavy chain diseases), and follicular lymphoma (e.g., Grades I, II, III, or IV).

In some embodiments, a malignancy treated using the methods of the present invention is a B-cell derived malignancy associated with the expression of one or more B-cell specific antigens such as, for example, CD3d, CD5, CD6, CD9, CD19, CD20, CD21, CD22, CD23, CD24, CD27, CD28, CD37, CD38, CD40, CD45, CD46, CD48, CD53, CD69, CD70, CD72, CD73, CD79a, CD79h, CD80, CD81, CD83, CD85a, CD85d, CD85e, CD85h, CD85i, CD85j, CD85k, CD86, CD96, CD98, CD100, CD121b, CD124, CD127, CD132, CD150, CD152, CD154, CD157, CD166, CD169, CD179a, CD179b, CD180, CD185, CD196, CD197, CD205, CDw2 0a, CD213a1, CD257, CD267, CD268, CD269, CD274, CD275, CD276, CD278, CD279, CD300a, CD300c, CD307, CD314, CD316, CD317, CD319, CD320, CDw327, and CD331. In a particular embodiment, a cancer treated using the methods of the invention is associated with the expression of CD-20. In another embodiment, a cancer treated using the methods of the invention is associated with the expression of CD-22. In yet another embodiment, a cancer treated using the methods of the invention is associated with the expression of both CD-20 and CD-22.

In some embodiments, a cancer treated using the methods of the invention is non-Hodgkin's lymphoma or NHL. Non-Hodgkin's lymphoma. or NHL, is a cancer of the lymphoid tissue which is formed by several types of immune cells including B-cells and T-cells. About 85% of the non-Hodgkin's lymphomas are derived from B-cells. NHL is thought to occur when B-cells, which produce antibodies, begin to grow abnormally. In some embodiments, non-Hodgkin's lymphoma treated using the methods of the invention is associated with the expression of CD-20 on B-cells. In other embodiments, non-Hodgkin's lymphoma is associated with the expression of CD-22. In yet other embodiments, non-Hodgkin's lymphoma is associated with the expression of both CD-20 and CD-22.

In some embodiments, a cancer treated using the methods and compositions of the invention is Hodgkin's lymphoma, also referred to as Hodgkin's disease. The cancer cells in Hodgkin's disease are called Reed-Sternberg cells, after the two doctors who first described them in detail. Under a microscope they look different from cells of non-Hodgkin's lymphomas and other cancers, and are believed to be a type of malignant B lymphocyte.

In some embodiments, a cancer treated using the methods and compositions of the invention is chronic lymphocytic leukemia (CLL) which is derived from a small B lymphocyte. CLL is mostly found in the blood and in the bone marrow.

In further embodiments, a cancer treated using the methods and compositions of the invention is mantle cell lymphoma.

In some embodiments, the B-cell malignancy is multiple myeloma, associated with uncontrolled proliferation of antibody producing cells in the plasma, which develop from B-cells.

In some embodiments, the B-cell malignancy is non-Hodgkin's lymphoma, chronic lymphocytic leukemia (CLL), small lymphocytic lymphoma, multiple myeloma, mantle cell lymphoma, B-cell prolymphocytic leukemia, lymphoplasmacytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, or Burkitt lymphoma/leukemia. In some embodiments, the B-cell malignancy is a mature B-cell lymphoma. In some embodiments, the mature B-cell lymphoma is B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, or Burkitt lymphoma/Burkitt cell leukemia.

In some embodiments, the mature B-cell lymphoma is a variant malignancy, for example, B-cell chronic lymphocytic leukemia/small lymphocytic lymphoma with monoclonal gammopathy/plasmacytoid differentiation, hairy cell leukemia variant, cutaneous follicle center lymphoma, diffuse follicle center lymphoma, blastoid mantle-cell lymphoma, morphologic variant of diffuse large B-cell lymphoma (for example, centroblastic, immunoblastic, T-cell/histiocyte-rich, lymphomatoid granulomatosis type, anaplastic large B-cell, plasmablastic) or subtype of diffuse large B-cell lymphoma (for example, mediastinal (thymic) large B-cell lymphoma, primary effusion lymphoma, intravascular large B-cell lymphoma), morphologic variant of Burkitt lymphoma or Burkitt cell leukemia (for example, Burkitt-like lymphoma/leukemia, Burkitt lymphoma/Burkitt cell leukemia with plasmacytoid differentiation (AIDS-associated), or clinical or genetic subtype of Burkitt lymphoma/Burkitt cell leukemia (for example, endemic, sporadic, immunodeficiency-associated).

In some embodiments, the epitope is a mimotope, which can be produced, for example, by phage display or by idiotypic antibody generation by immunization of an animal. For example, a hybridoma cell-line may be developed which contains a tumor-associated antigen (optionally, a tumor-specific antigen) obtain from a patient, which is unique to that patient and found exclusively on the surface of a B-lymphocyte associated with a B-cell derived cancer such as, for example, non-Hodgkin's lymphoma, Hodgkin's lymphoma, chronic lymphocytic leukemia, mantle cell lymphoma or multiple myeloma, and which is absent or expressed in decreased amounts in normal B-lymphocytes and other cells.

In some embodiments, an “autologous idiotype vaccine” includes an epitope or antigen associated with a B-cell derived cancer in a subject (for example, non-Hodgkin's lymphoma, Hodgkin's lymphoma, chronic lymphocytic leukemia, mantle cell lymphoma or multiple myeloma) linked to a carrier molecule, such as a carrier protein. Preferably, the carrier molecule is immunogenic, such as the immunogenic carrier protein KLH ((keyhole limpet hemocyanin) Kwak L W et al., N Engl. J. Med., 327:1209-1215 (1992); Hsu F J et al., Blood, 89:3129-3135 (1997); Schumacher K, J. Cancer Res. Clin. Oncol., 127(Suppl 2):R1-R2 (2001)).

In some embodiments, a mimotope is associated with a B-cell derived malignancy in the subject, and the antigen is produced by a hybridoma (e.g., by hybridoma rescue fusion hybridization; see, for example, Lee S T et al., Expert Opin Biol Ther, 7(1):113-122 (2007); Flowers C R, Expert Rev Vaccines, 6(3):307-317 (2007); Neelapu S S and L W Kwak, Hematology, 243-249, (2007); Lee S-T. et al., Yonsei Medical Journal, 48(1):1-10 (2007); Ruffini P A et al., Haematologica, 87:989-1001 (2002), which are each incorporated herein by reference in their entirety). In some embodiments, the hybridoma is produced by fusion of a cancerous B-cell obtained from the subject and a murine/human heterohybridoma myeloma cell, such as the K6H6/B5 cell line. In some embodiments, the antigen-producing hybridoma is grown in a hollow-fiber bioreactor, such as those described in one or more of International Patent Publications WO 2007/139748 (Biovest International, Inc., filed May 21, 2007); WO 2007/139742 (Biovest International, Inc., filed May 21, 2007); WO 2007/139746 (Biovest International, Inc., filed May 21, 2007); WO 2007/136821 (Biovest International, Inc., filed May 21, 2007); and WO 2007/139747 (Biovest International, Inc., filed May 21, 2007), each of which are incorporated herein by reference in their entirety). The antigen can then be collected from the hollow-fiber bioreactor and purified (e.g., by affinity chromatography) prior to administration to the subject.

Samples of malignant cells (e.g., tumor cells) can be obtained from a subject for isotyping by biopsy, fine-needle aspiration, or apheresis, for example. The immunoglobulin to be isotyped may be present on the malignant cell surface, within the malignant cell cytoplasm, or in the subject's blood. The method of collection will depend upon where the immunoglobulin-bearing cells or secreted immunoglobulin molecules are found. For example, depending upon the malignancy, samples can be obtained from lymph nodes, extra-nodal tissue, spleen, bone marrow, or blood (Alvarez-Vallina L. et al., Journal of Immunotherapy, 1995, 17:194-198).

Malignant cells can be isotyped by flow cytometry (Zabelegui N. et al., haeamatologica, 2004, 89(5):541-546). Antibodies specific for various isotypes are commercially available. For example, human anti-IgM antibodies are available from Miltenyi Biotec (Auburn Calif.). Other methods such as immunofluoroescence, immunohistochemistry of sections (e.g., from a biopsy), sequencing of the constant region on the heavy chain, immunoblot, etc. (Fakhrjou A. et al., Pakistan Journal of Biological Sciences, 2010, 13 (4):194-197).

In some embodiments, the B-cell malignancy exhibits a predetermined immunoglobulin isotype or isotypes that is not an IgM isotype (a non-IgM immunoglobulin). In some embodiments, the B-cell the malignancy exhibits a predetermined immunoglobulin isotype or isotypes that is an IgM isotype (an IgM immunoglobulin). In some embodiments, the non-IgM immunoglobulin is IgG, IgA, IgD, IgE, or any combination of two or more of the foregoing (for example, IgM/IgA or IgM/IgG). In some embodiments, the non-IgM immunoglobulin is IgG1, IgG2, IgG3, IgG4, IgA1, IgA2, IgE, IgD, or any combination of the foregoing.

Accordingly, in some embodiments, the immunoglobulin isotype or isotypes exhibited by the malignancy represents an immunoglobulin that is present on the malignant cell (surface), within the malignant cell, secreted by the malignancy or is found in the subject's blood, or any combination of two or more of the foregoing, and, optionally, the immunoglobulin isotype or isotypes exhibited by the malignancy is predetermined. For example, the immunoglobulin isotype or isotypes exhibited by the malignancy may be predetermined by obtaining a tumor, tissue or blood sample from the subject by biopsy (e.g., surgical biopsy or needle biopsy), needle aspiration, or apheresis. In some embodiments, the immunoglobulin isotype or isotypes exhibited by the malignancy is predetermined by obtaining a sample of lymph node tissue, extra-nodal tissue, spleen, bone marrow, or blood. In some embodiments, the immunoglobulin isotype or isotypes exhibited by the malignancy is predetermined by flow cytometry, immunofluoroescence, sequencing of heavy chain constant region, or immunoblot.

### VI. MODES OF ADMINISTRATION

The various sensitizing and tolerizing agents (one or more epitopes and either an IgM Fc region or an IgG Fc region; nucleic acid molecules encoding epitopes and/or Fc regions) used in the compositions and methods described herein may be administered orally, parenterally (e.g., intravenously), intramuscularly, sublingually, buccally, rectally, intranasally, intrabronchially, intrapulmonarily, intraperitoneally, topically, transdermally and subcutaneously, for example. The amount administered in a single dose may be dependent on the subject being treated, the subject's weight, the manner of administration and the judgment of the prescribing physician. Generally, however, administration and dosage and the duration of time for which a composition is administered will approximate that which are necessary to achieve a desired result.

Single or multiple administrations of the sensitizing and tolerizing agents can be carried out with dose levels and pattern being selected by the treating physician. In any event, the compositions should comprise a quantity of sensitizing or tolerizing agents sufficient to effectively sensitize or tolerize the subject as desired.

In general, a therapeutically effective amount of a monoclonal antibody such as, for example, an antibody that specifically binds CD-20 or CD-22, can be from about 0.0001 mg/Kg to 0.001 mg/Kg; 0.001 mg/kg to about 10 mg/kg body weight or from about 0.02 mg/kg to about 5 mg/kg body weight. In some embodiments, a therapeutically effective amount of a monoclonal antibody is from about 0.001 mg to about 0.01 mg, about 0.01 mg to about 100 mg, or from about 100 mg to about 1000 mg, for example.

In some embodiments, a therapeutically effective amount of an autologous idiotype vaccine is from about 0.001 mg to about 0.01 mg, about 0.01 mg to about 100 mg, or from about 100 mg to about 1000 mg, for example. In some embodiments, an effective amount of the autologous idiotype vaccine is one or more doses of 0.5 mg.

In some embodiments, an effective amount of an antibody administered to a subject having non-Hodgkin's lymphoma, Hodgkin's lymphoma, chronic lymphocytic leukemia or multiple myeloma is between about 100 mg/m² and 200 mg/m², or between about 200 mg/m² and 300 mg/m² or between about 300 mg/m² and 400 mg/m². In a particular embodiment, an effective amount of a monoclonal antibody that selectively binds a B-cell specific antigen is about 375 mg/m².

The optimal pharmaceutical formulations for a sensitizing or tolerizing formulation can be readily determined by one or ordinary skilled in the art depending upon the route of administration and desired dosage. (See, for example, Remington's Pharmaceutical Sciences, 18th Ed. (1990), Mack Publishing Co., Easton, Pa., the entire disclosure of which is hereby incorporated by reference).

The epitopes and Fc constant regions described herein can be formulated for the most effective route of administration, including for example, oral, transdermal, sublingual, buccal, parenteral, rectal, intranasal, intrabronchial or intrapulmonary administration.

In some embodiments, the sensitizing methods of the present invention include one or more cytokines such as, for example, GM-CSF, or other immunostimulatory agents. GM-CSF is a potent immunostimulatory cytokine with efficacy in promoting anti-tumor response, particularly T cell responses. In general, however, any cytokine or chemokine that induces inflammatory responses, recruits antigen presenting cells (APC) to the tumor and, possibly, promotes targeting of antigen presenting cells (APC) may be used, for example.

The epitopes and Fc constant regions (IgM Fc region or IgG Fc region) useful in the methods of the present invention may be administered by any conventional route including oral and parenteral. Examples of parenteral routes are subcutaneous, intradermal, transcutaneous, intravenous, intramuscular, intraorbital, intracapsular, intrathecal, intraspinal, intracisternal, intraperitoneal, etc. If booster doses are utilized, the primary treatment and one or more booster doses are preferably administered by the same route, e.g., subcutaneously.

The antigen and the Fc constant region (IgM Fc region or IgG Fc region) can be administered within the same formulation or different formulations. If administered in different formulations, the antigen and the Fc constant region can be administered by the same route or by different routes. Administration is preferably by injection on one or multiple occasions to produce systemic immunity. In general, multiple administrations in a standard immunization protocol are used, as is standard in the art. For example, the vaccines can be administered at approximately two to six week intervals, or monthly, for a period of from one to six inoculations in order to provide protection. The vaccine may be administered by any conventional route including oral and parenteral. Examples of parenteral routes are subcutaneous, intradermal, transcutaneous, intravenous, intramuscular, intraorbital, intracapsular, intrathecal, intraspinal, intracisternal, intraperitoneal, etc.

Without wishing to be bound by theory, it is contemplated that sensitization may result in a systemic immune response, which includes either or both of an antibody response and a cell-mediated immune response, which will provide a clinical therapeutic effect and/or result in antibodies and activated T lymphocytes of various classes which may be used themselves as therapeutic agents, for example, for producing passive immunity in subjects.

The sensitizing compositions used in the methods of the present invention may further include one or more adjuvants or immunostimulatory agents. Examples of adjuvants and immunostimulatory agents include, but are not limited to, aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives, liposomes, levamisole, DEAE-dextran, blocked copolymers or other synthetic adjuvants. Such adjuvants are readily commercially available.

Depending on the intended mode of administration, the compounds used in the methods described herein may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. Each dose may include an effective amount of a compound used in the methods described herein in combination with a pharmaceutically acceptable carrier and, in addition, may include other medicinal agents, pharmaceutical agents, carriers, adjuvants, diluents, etc.

Liquid pharmaceutically administrable compositions can prepared, for example, by dissolving, dispersing, etc., a compound for use in the methods described herein and optional pharmaceutical adjuvants in an excipient, such as, for example, water, saline aqueous dextrose, glycerol, ethanol, and the like, to thereby form a solution or suspension. For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; see, for example, Remington's Pharmaceutical Sciences, 18th Ed. (1990), Mack Publishing Co., Easton, Pa., the entire disclosure of which is hereby incorporated by reference).

Formulations comprising sensitizing and tolerizing agents may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

### VII. VECTORS AND CONSTRUCTS

In some embodiments, the sensitization method of the invention comprises administering a nucleic acid molecule encoding the epitope and the IgM Fc region, wherein the nucleic acid molecule is expressed in the subject to produce the epitope and the IgM Fc region separately or as a fusion polypeptide. Likewise in some embodiments, the tolerization method of the invention comprises administering a nucleic acid molecule encoding the epitope and the IgG Fc region, and wherein the nucleic acid molecule is expressed to produce the epitope and the IgG Fc region separately or as a fusion polypeptide.

Methods of introducing nucleic acids such as DNA vaccines into individuals are well-known to the skilled artisan. For example, nucleic acids can be injected into skeletal muscle or other somatic tissues (e.g., intramuscular injection). Cationic liposomes or biolistic devices, such as a gene gun, can be used to deliver nucleic acids. Alternatively, iontophoresis and other means for transdermal transmission can be used for the introduction of nucleic acids into an individual.

Thus, the present invention also relates to vectors and to constructs that include nucleic acid sequences that may be transcribed and/or translated to yield epitopes and Fc constant regions; to host cells which are genetically engineered with vectors and/or constructs of the invention and to the production of such vectors, constructs, and host cells. Nucleic acid sequences encoding epitopes and Fc constant regions may be engineered to produce the corresponding polypeptides using well-established methodologies such as those described herein.

According to the present invention, a vector may comprise a recombinant nucleic acid construct containing one or more promoters for transcription of nucleic acid sequences encoding epitopes and Fc constant regions.

Nucleic acid molecules of the invention can be expressed in mammalian cells, yeast, bacterial cells, insect cells, plant cells, viral cells, fungal cells, or other cells under the control of appropriate promoters (see, for example, Bendandi, M. et al., “Rapid, high-yield production in plants of individualized idiotype vaccines for non-Hodgkin's lymphoma,” Ann Oncol., 21(12):2420-2427 (2010); Bertinetti, C. et al., “Cloning of idiotype immunoglobulin genes in B cell lymphomas by anchored PCR and production of individual recombinant idiotype vaccines in Escherichia coli,” Eur J Haematol, 77(5):395-402 (2006); Tchoudakova, A. et al., “High level expression of functional human IgMs in human PER.C6 cells,” MAbs, (2):163-71 (2009); Wood, C. R. et al., “High level synthesis of immunoglobulins in Chinese hamster ovary cells,” J Immunol, 145(9): p. 3011-6 (1990)). Appropriate cloning and expression vectors for use with prokaryotic and eukaryotic hosts are described, for example, by Sambrook, et al., Molecular Cloning: A Laboratory Manual, Third Edition, Cold Spring Harbor, N.Y., (2001).

The appropriate nucleic acid sequence(s) may be inserted into the vector by a variety of procedures. In general, the nucleic acid sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (2001 Molecular Cloning, Third Ed., Cold Spring Harbor Laboratory, Plainview, N.Y.); Maniatis et al. (1982 Molecular Cloning, Cold Spring Harbor Laboratory, Plainview, N.Y.); and elsewhere.

The nucleic acid sequence in the expression vector is generally operatively linked to at least one appropriate expression control (i.e., regulatory) sequence (e.g., a promoter or a regulated promoter) to direct mRNA synthesis. Selection of the appropriate vector and promoter is well within the level of ordinary skill in the art, and preparation of certain particularly preferred recombinant expression constructs comprising at least one promoter, or regulated promoter, operably linked to a nucleic acid described herein.

In some embodiments the vector is a viral vector such as a mammalian viral vector (e.g., retrovirus, adenovirus, adeno-associated virus, lentivirus). The viral vector can include one or more promoters. Suitable promoters that may be employed include, but are not limited to, the retroviral LTR; the SV40 promoter; and the human cytomegalovirus (CMV) promoter described in Miller, et al., Biotechniques 7:980-990 (1989), or any other promoter (e.g., cellular promoters such as eukaryotic cellular promoters including, but not limited to, the histone, pol III, and beta-actin promoters). Other viral promoters that may be employed include, but are not limited to, adenovirus promoters, adeno-associated virus promoters, thymidine kinase (TK) promoters, and B19 parvovirus promoters. The selection of a suitable promoter will be apparent to those skilled in the art from the teachings contained herein, and may be from among either regulated promoters (e.g., tissue-specific or inducible promoters) or promoters as described above. A tissue-specific promoter allows preferential expression of the nucleic acid in a given target tissue, thereby avoiding expression in other tissues. For example, to express nucleic acids specifically in the heart, a number of cardiac-specific regulatory elements can be used. An example of a cardiac-specific promoter is the ventricular form of MLC-2v promoter (see, Zhu et al., Mol. Cell. Biol. 13:4432-4444, 1993; Navankasattusas et al., Mol. Cell. Biol. 12:1469-1479, 1992) or a variant thereof such as a 281 bp fragment of the native MLC-2v promoter (nucleotides −264 to +17, Genebank Accession No. U26708). Examples of other cardiac-specific promoters include alpha myosin heavy chain (Minamino et al., Circ. Res. 88:587-592, 2001) and myosin light chain-2 (Franz et al., Circ. Res. 73:629-638, 1993). Endothelial cell gene promoters include endoglin and ICAM-2. See Velasco et al., Gene Ther. 8:897-904, 2001. Liver-specific promoters include the human phenylalanine hydroxylase (PAH) gene promoters (Bristeau et al., Gene 274:283-291, 2001), hB1F (Zhang et al., Gene 273:239-249, 2001), and the human C-reactive protein (CRP) gene promoter (Ruther et al., Oncogene 8:87-93, 1993). Promoters that are kidney-specific include CLCN5 (Tanaka et al., Genomics 58:281-292, 1999), renin (Sinn et al., Physical Genomics 3:25-31, 2000), androgen-regulated protein, sodium-phosphate cotransporter, renal cytochrome P-450, parathyroid hormone receptor and kidney-specific cadherin. See Am. J. Physiol. Renal Physiol. 279:F383-392, 2000. An example of a pancreas-specific promoter is the pancreas duodenum homeobox 1 (PDX-1) promoter (Samara et al., Mol. Cell. Biol. 22:4702-4713, 2002). A number of brain-specific promoters may be useful in the invention and include the thy-1 antigen and gamma-enolase promoters (Vibert et al., Eur. J. Biochem. 181:33-39, 1989), the glial-specific glial fibrillary acidic protein (GFAP) gene promoter (Cortez et al., J. Neurosci. Res. 59:39-46, 2000), and the human FGF1 gene promoter (Chiu et al., Oncogene 19:6229-6239, 2000). The GATA family of transcription factors has promoters directing neuronal and thymocyte-specific expression (see Asnagli et al., J. Immunol. 168:4268-4271, 2002).

Nucleic acids can be administered to a subject by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in Hamajima et al., Clin. Immunol. Immunopathol. 88(2):205-10 (1998). In addition to viral-mediated nucleic acid delivery, other techniques for delivery of nucleic acids may be employed. For example, non-viral vectors may be used to deliver nucleic acid constructs encoding epitopes and/or Fc constant regions, resulting in expression. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In the treatment of cancer, for example, various methodologies and vectors available for delivering and expressing nucleic acids in vivo are known (Robson et al., J. Biomed and Biotechnol., 2003, 2003(2):110-137). Various targeting techniques are available, including transcriptional targeting using tissue-specific and event-specific transcriptional control elements, and tumor-specific promoters, tumor environment-specific promoters, and exogenously controlled inducible promoters.

In another aspect, the present invention relates to host cells containing the above described recombinant constructs. Host cells are genetically engineered/modified (transduced, transformed or transfected) with the vectors and/or expression constructs of this invention that may be, for example, a cloning vector, a shuttle vector, or an expression construct. The vector or construct may be, for example, in the form of a plasmid, a viral particle, a phage, etc. The engineered host cells can be cultured in conventional nutrient media modified as appropriate for activating promoters, selecting transformants or amplifying particular nucleic acids encoding epitopes and/or Fc constant regions, or fusion polypeptides thereof. The culture conditions for particular host cells selected for expression, such as temperature, pH and the like, will be readily apparent to the ordinarily skilled artisan.

The host cell can be a higher eukaryotic cell, such as a mammalian cell, or a lower eukaryotic cell, such as a yeast cell, or the host cell can be a prokaryotic cell, such as a bacterial cell. Representative examples of appropriate host cells according to the present invention include, but need not be limited to, bacterial cells, such as E. coli, Streptomyces, Salmonella typhimurium; fungal cells, such as yeast; insect cells, such as Drosophila S2 and Spodoptera Sf9; animal cells, such as CHO, COS or 293 cells; adenoviruses; plant cells, or any suitable cell already adapted to in vitro propagation or so established de novo.

Various mammalian cell culture systems can also be employed to produce epitopes and Fc constant regions. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa, HEK, and BHK cell lines. Mammalian expression vectors will typically comprise an origin of replication, a suitable promoter and enhancer, and also any necessary ribosome binding sites, polyadenylation site, splice donor and acceptor sites, transcriptional termination sequences, and 5′ flanking nontranscribed sequences, for example, for the preparation of recombinant nucleic acid constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including but not limited to, for example, liposomes including cationic liposomes, calcium phosphate transfection, DEAE-Dextran mediated transfection, or electroporation (Davis et al., 1986 Basic Methods in Molecular Biology), or other suitable technique.

The expressed nucleic acids may be useful in intact host cells; in intact organelles such as cell membranes, intracellular vesicles or other cellular organelles; or in disrupted cell preparations including but not limited to cell homogenates or lysates, microsomes, uni- and multilamellar membrane vesicles or other preparations. Alternatively, expressed nucleic acids can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Finally, high performance liquid chromatography (HPLC) can be employed for final purification steps.

In some embodiments, the invention provides a multi-epitope construct comprising: 1) nucleic acids that encode multiple epitopes (of any length); or 2) polypeptides comprising multiple polypeptide epitopes. Some embodiments provide for “multi-epitope constructs” that comprise a combination or series of different epitopes, optionally connected by flanking residues. Multi-epitope constructs can further comprise an IgM Fc region or a nucleic acid sequence encoding the IgM Fc region (for sensitization), or an IgG Fc region or nucleic acid sequence encoding the IgG Fc region (for tolerization).

Multi-epitope constructs can, optionally, contain flanking or spacing residues between each epitope. Some embodiments provide for multi-epitope constructs that comprise a series of the same epitope (termed “homopolymers”). Other embodiments provide for multi-epitope constructs that comprise a combination or series of different epitopes, optionally connected by flanking or spacing residues (termed “heteropolymers”). In some embodiments, in cases in which antigens are proteinacious, multi-epitope constructs may exclude amino acid residues from antigens from which the epitopes are obtained. Thus, optionally, multi-epitope constructs can include the full antigen or exclude regions of the antigen that are outside of the epitopic region, or exclude other epitopes of the antigen.

A “flanking” or “linking” residue is a residue that is positioned next to an epitope. A flanking residue can be introduced or inserted at a position adjacent to the N-terminus or the C-terminus of an epitope. Flanking residues suitable for use in the subject invention are disclosed, for example, in U.S. Pat. No. 6,419,931, which is hereby incorporated by reference in its entirety, including all sequences, figures, references, and tables.

A “spacer” or “linker” refers to a sequence that is inserted between two epitopes in a multi-epitope construct to prevent the occurrence of junctional epitopes and/or to increase the efficiency of processing. A multi-epitope construct may have one or more spacer nucleic acids. A spacer nucleic acid may flank each epitope nucleic acid in a construct, or the spacer nucleic acid to epitope nucleic acid ratio may be about 2 to 10, about 5 to 10, about 6 to 10, about 7 to 10, about 8 to 10, or about 9 to 10, where a ratio of about 8 to 10 has been determined to yield favorable results for some constructs. The spacer nucleic acid may encode one or more amino acids.

In some multi-epitope constructs, it is sufficient that each spacer nucleic acid encodes the same amino acid sequence. In multi-epitope constructs having two spacer nucleic acids encoding the same amino acid sequence, the spacer nucleic acids encoding those spacers may have the same or different nucleotide sequences, where different nucleotide sequences may be preferred to decrease the likelihood of unintended recombination events when the multi-epitope construct is inserted into cells.

### VIII. ASSESSING IMMUNE RESPONSE

The methods of the invention may further comprise, after administering the epitope and the appropriate Fc region, verifying whether the subject has been sensitized or tolerized to the epitope. The methods of the invention may comprise assessing whether an immune response to the epitope has been elicited in the subject and, optionally, determining whether the immune response against the epitope has subsequently increased, diminished, or remained the same (e.g., in character and/or extent).

An assessment can be made of the nature and/or extent of the subject's immune response to the epitope (e.g., cellular and/or humoral response) one or more times after the initial treatment. Preferably, an assessment of the subject's immune response is also made before the subject's initial treatment (e.g., to establish a control or base-line for comparison to a subsequent assessment or assessments post-treatment).

If the subject has a B-cell malignancy, when assessing the subject's immune response. the immune response against the B-cell idiotype is preferably assessed. However, the assessment can include an assessment of the subject's immune response against any component of the formulation. An assessment of the subject's immune response against the anti-idiotype, or against a carrier molecule (e.g., KLH), or against both, can be made. For example, enzyme-linked immunosorbent assays (ELISA) and/or T-cell proliferation assays can be performed for detection of, for example, anti-Id humoral and/or cellular responses after vaccination (Hsu F. J. et al., “Tumor-specific idiotype vaccines in the treatment of patients with B-cell lymphoma—long term results of a clinical study,” Blood, 1997, 89:3129-3135).

The subject's immune response to the administered epitope can be monitored by making multiple assessments after the initial treatment at uniform time intervals (e.g., every three months, every six months, every nine months, or annually) or at non-uniform time intervals. Monitoring of the subject's immune response to the administered epitope can continue for a pre-determined period of time, for a time determined based on therapeutic outcome, or indefinitely. Preferably, the subject's immune response is monitored from a time period starting prior to initial vaccination and continuing for a period of at least five years, or indefinitely.

Typically, each assessment will involve obtaining an appropriate biological sample from the subject. The appropriate biological sample will depend upon the particular aspect of the subject's immune response to be assessed (e.g., depending upon the particular assay). For example, in some embodiments, the biological sample will be one or more specimens selected from among blood, peripheral blood mononuclear cells (PBMC), and a tumor. Samples for assessments are taken at a time point appropriate to obtain information regarding the immune response at the time of interest. For example, a sample may be taken from the subject from a time prior to administration of the epitope and additional samples may be taken from the subject periodically after administration to determine the nature and extent of the immune responses observed.

In some embodiments, in the case of a B-cell malignancy, assessment of the immune response includes assessment of one or more of the following aspects of the immune response: anti-idiotype (anti-Id) humoral responses; tumor-specific antibodies; tumor-reactive T-cell precursor frequencies (e.g., via an IFN-gamma response); biomarkers in the B-cell derived tumor that correlate with clinical outcome following autologous anti-idiotype vaccine therapy; and B-cell derived tumor-specific CD4+ and CD8+ T-cell responses.

Preferably, the immune response is assessed by conducting one or more humoral response assays and/or cellular response assays, such as those described by Neelapu et al. (Nature Medicine, 11(9):986-991 (2005)), which is incorporated herein by reference in its entirety. Peripheral blood B and T cells can be collected from the subject and blood counts can be determined, including but not limited to CD3−CD19+ B cells, CD3+ CD4+ T cells, and CD3+CD8+ T cells. Tumor cells can be determined, and PBMCs isolated. Both B-cells and tumor cells can be activated with recombinant CD40 ligand trimer, as described in Neelapu et al. (2005). Depending on the type of immune response to be assessed (e.g., humoral, cellular, or both), one or more of the following assays may be used:
Humoral immune response assay: to assess anti-Id humoral responses and tumor-specific antibodies (see, for example, Kwak et al., Lancet, 345:1016-1020 (1995), which is incorporated herein by reference in its entirety).
IFN-gamma ELISPOT assay: to assess tumor-reactive T-cell precursor frequencies via an IFN-gamma response (see, for example, Malyguine et al., J. Trans. Med., 2:9 (2004) and Neelapu et al., Clin. Cancer Res., 10:8309-8317 (2004), which are each incorporated herein by reference in its entirety).
Cytokine induction assay: to assess biomarkers in the tumor that correlate with clinical outcome following autologous anti-idiotype vaccine therapy (see, for example, Neelapu et al. (2004)).
Intracellular cytokine assay: to assess tumor-specific CD4+ and CD8+ T-cell responses (Neealapu et al., J. Cancer Res. Clin. Oncol., 127 Suppl. 2, R14-19 (2001)).

### IX. DEFINITIONS

In order that the present disclosure may be more readily understood, certain terms are first defined. Additional definitions are set forth throughout the detailed description.

As used herein, the term “co-administering” and grammatical variations thereof, is intended to mean administration of two or more agents to a subject simultaneously or sequentially (in any order), within the same formulations or in different formulations. For example, one or more epitopes (in isolation or as part of an intact antigen) can be co-administered to a subject with an IgM Fc region to sensitize a subject to the epitope. Likewise, one or more epitopes (in isolation or as part of an intact antigen) can be co-administered to a subject with an IgG Fc region to tolerize the subject to the epitope.

As used herein, the term “sensitizing” refers to inducing or increasing a humoral and/or cellular immune response against an epitope (for example, a polypeptide) in the subject.

As used herein, the term “tolerizing” refers to reducing (eliminating or suppressing) a humoral and/or cellular immune response against an epitope in the subject.

As used herein, the term “antigen” refers to a molecule (for example, a polypeptide, nucleic acid molecule, carbohydrate, glycoprotein, lipid, lipoprotein, glycolipid, or small molecule) that is capable of eliciting an immune response and contains an epitope or antigenic determinant to which an immunoglobulin can specifically bind.

As used herein, the term “epitope” or “antigenic determinant” or “idiotypic determinant” refers to a site on an antigen to which an immunoglobulin (or antigen binding fragment thereof) can specifically bind. Epitopes can be formed both from contiguous amino acids or noncontiguous amino acids juxtaposed by tertiary folding of a protein. Epitopes found on the Fab (variable) region of immunoglobulins are referred to as “idiotypic determinants” and comprise the immunoglobulin's “idiotype”. Epitopes formed from contiguous amino acids are typically retained on exposure to denaturing solvents, whereas epitopes formed by tertiary folding are typically lost on treatment with denaturing solvents. In the case of proteinaceous antigens, an epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, for example, Epitope Mapping Protocols in Methods in Molecular Biology, Vol. 66, G. E. Morris, Ed. (1996).

An epitope “involved in” or “associated with” a disorder includes an epitope, the normal or aberrant production or function of which affects or causes a disease or disorder or at least one symptom of the disease or disorder. For example, the A-beta peptide is associated with Alzheimer's disease, and PrP^{Sc} is associated with prion disease.

The term “domain” refers to a globular region of a heavy or light chain polypeptide comprising peptide loops (e.g., comprising 3 to 4 peptide loops) stabilized, for example, by beta-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. “Constant” domains on the light chain are referred to interchangeably as “light chain constant regions”, “light chain constant domains”, “CL” regions or “CL” domains). “Constant” domains on the heavy chain are referred to interchangeably as “heavy chain constant regions”, “heavy chain constant domains”, “CH” regions or “CH” domains). “Variable” domains on the light chain are referred to interchangeably as “light chain variable regions”, “light chain variable domains”, “VL” regions or “VL” domains). “Variable” domains on the heavy chain are referred to interchangeably as “heavy chain variable regions”, “heavy chain variable domains”, “VH” regions or “VH” domains).

The term “region” refers to a part or portion of an antibody chain or antibody chain domain (for example, a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein. As used herein, a “region” of an antibody is inclusive of regions existing in isolation (as antibody fragments) and as part of whole (intact) or complete antibodies.

As used herein, the teens “constant region” or “fragment crystallizable region” (Fc region) refers to that portion of the antibody (the tail region) that interacts with cell surface receptors called Fc receptors and some proteins of the complement system, and is composed of two heavy chains that contribute two or three constant domains depending on the class of the antibody (Janeway C A, Jr et al. (2001). Immunobiology. (5th ed.). Garland Publishing). In IgG, IgA and IgD antibody isotypes, the Fc region is composed of two identical protein fragments, derived from the second and third constant domains of the antibody's two heavy chains; IgM and IgE Fc regions contain three heavy chain constant domains (C_{H} domains 2-4) in each polypeptide chain. The Fc regions of IgGs bear a highly conserved. N-glycosylation site (Janeway C A, Jr et al. (2001). Immunobiology. (5th ed.); Garland Publishing Rhoades R A, Pflanzer R G (2002). Human Physiology (4th ed.). Thomson Learning). The other part of an antibody, called the Fab region, contains variable sections that define the specific target that the antibody can bind. By contrast, the Fc region of all antibodies in a class is the same for each species; they are constant rather than variable. The terms “Fc region” and “Fab region” encompass these regions existing in isolation (as antibody fragments) and as part of a whole (intact) or complete, full-length antibody.

As used herein, the term “antibody” is used interchangeably with “immunoglobulin” or “Ig,” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity or functional activity (sensitizing activity or tolerizing activity). Single chain antibodies, and chimeric, human, humanized or primatized (CDR-grafted) antibodies, as well as chimeric or CDR-grafted single chain antibodies, and the like, comprising portions derived from different species, are also encompassed by the present invention and the term “antibody”. The various portions of these antibodies can be joined together chemically by conventional techniques, synthetically, or can be prepared as a contiguous protein using genetic engineering techniques. For example, nucleic acids encoding a chimeric or humanized chain can be expressed to produce a contiguous protein. See, e.g., U.S. Pat. No. 4,816,567; European Patent No. 0,125,023 B1; U.S. Pat. No. 4,816,397; European Patent No. 0,120,694 B1; WO 86/01533; European Patent No. 0,194,276 B1; U.S. Pat. No. 5,225,539; European Patent No. 0,239,400 B1 and U.S. Pat. Nos. 5,585,089 and 5,698,762. See also, Newman, R. et al. BioTechnology, 10: 1455-1460, 1993, regarding primatized antibody, and Ladner et al., U.S. Pat. No. 4,946,778 and Bird, R. E. et al., Science, 242:423-426, 1988, regarding single chain antibodies. It is understood that all forms of the antibodies comprising an Fc region (or portion thereof) are encompassed herein within the term “antibody.” Furthermore, the antibody may be labeled with a detectable label, immobilized on a solid phase and/or conjugated with a heterologous compound (e.g., an enzyme or toxin) according to methods known in the art. The term “antibody” encompasses whole antibodies as well as antibody fragments.

As used herein, the term “antibody fragments” refers to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

The terms “polynucleotide”, “nucleic acid molecule”, and “nucleic acid” are used interchangeably herein to refer to a polymeric form of nucleotides of any length, which contain deoxyribonucleotides, ribonucleotides, and analogs in any combination analogs. Polynucleotides may have any three-dimensional structure, and may perform any function, known or unknown. The term “nucleic acid molecule” includes double-, single-stranded, and triple-helical molecules. Unless otherwise specified or required, any embodiment of the invention described herein that is a nucleic acid molecule encompasses both the double-stranded form and each of two complementary single-stranded forms known or predicted to make up the double stranded form. In some embodiments, the nucleic acid molecule encodes an epitope or an antigen.

The following are non-limiting examples of nucleic acid molecules: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A nucleic acid molecule may be further modified after polymerization, such as by conjugation with a labeling component. Other types of modifications included in this definition are caps, substitution of one or more of the naturally occurring nucleotides with an analog, and introduction of means for attaching to proteins, metal ions, labeling components, other nucleic acid molecules, or a solid support.

The terms “polypeptide”, “peptide” and “protein” are used interchangeably herein to refer to polymers of amino acids of any length. The polymer may be linear or branched, it may comprise modified amino acids or amino acid analogs, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component.

The terms “fusion polypeptide” and “fusion protein” refer to a polypeptide comprising regions in a different position in the sequence than occurs in nature. The regions may normally exist in separate proteins and are brought together in the fusion polypeptide; or they may normally exist in the same protein but are pieced in a new arrangement in the fusion polypeptide. Fusion polypeptides can be produced by linking two or more polypeptides together (for example, covalently), or by expressing nucleic acids encoding each fusion partner within a host cell, for example. In some embodiments of the invention, a fusion polypeptide comprising an epitope and an IgM Fc region (for sensitization), or comprising an epitope and an IgG Fc region (for tolerization) are administered to a subject. The fusion polypeptide may be administered to the subject as a polypeptide or as a nucleic acid encoding the fusion polypeptide.

The term “adjuvant” refers to a substance co-administered with an antigen (e.g., incorporated into or administered simultaneously with an antigen) which potentiates the immune response in response to that antigen but does not in itself confer immunity. A tetanus, diphtheria, and pertussis vaccine, for example, contains minute quantities of toxins produced by each of the target bacteria, but also contains some aluminum hydroxide. Aluminum salts are common adjuvants in vaccines sold in the United States and have been used in vaccines for over 70 years. The body's immune system develops an antitoxin to the bacteria's toxins, not to the aluminum, but would not respond enough without the help of the aluminum adjuvant. An adjuvant can also include cytokines such as granulocyte-monocyte colony stimulating factor (GM-CSF). In some cases, e.g., immunization of a subject against normally non-immunogenic tumor-derived idiotypes, foreign (non-self) carrier protein immunogens such as keyhole limpet hemocyanin (KLH), can also potentiate the immune response and serve as adjuvants.

The terms “B lymphocyte” and “B cell,” as used interchangeably herein, are intended to refer to any cell within the B cell lineage as early as B cell precursors, such as pre-B cells B220⁺ cells which have begun to rearrange Ig VH genes and up to mature B cells and even plasma cells such as, for example, plasma cells which are associated with multiple myeloma. The term “B-cell,” also includes a B-cell derived cancer stem cell, i.e., a stem cell which is capable of giving rise to non-Hodgkin's lymphoma, Hodgkin's lymphoma, chronic lymphocytic leukemia, mantle cell lymphoma or multiple myeloma. Such cells can be readily identified by one of ordinary skill in the art using standard techniques known in the art and those described herein.

The terms “B-cell malignancy” and “B-cell derived malignancy” are used interchangeably herein to refer to a malignancy arising from aberrant replication of B cells. B-cell malignancies include, for example, non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, Burkitt lymphoma/leukemia. The B-cell malignancy may be a mature B-cell lymphoma. Examples of mature B-cell lymphomas include B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, Burkitt lymphoma/Burkitt cell leukemia.

The mature B-cell lymphoma may be a variant malignancy, for example, B-cell chronic lymphocytic leukemia/small lymphocytic lymphoma with monoclonal gammopathy/plasmacytoid differentiation, hairy cell leukemia variant, cutaneous follicle center lymphoma, diffuse follicle center lymphoma, blastoid mantle-cell lymphoma, morphologic variant of diffuse large B-cell lymphoma (for example, centroblastic, immunoblastic, T-cell/histiocyte-rich, lymphomatoid granulomatosis type, anaplastic large B-cell, plasmablastic) or subtype of diffuse large B-cell lymphoma (for example, mediastinal (thymic) large B-cell lymphoma, primary effusion lymphoma, intravascular large B-cell lymphoma), morphologic variant of Burkitt lymphoma or Burkitt cell leukemia (for example, Burkitt-like lymphoma/leukemia, Burkitt lymphoma/Burkitt cell leukemia with plasmacytoid differentiation (AIDS-associated), or clinical or genetic subtype of Burkitt lymphoma/Burkitt cell leukemia (for example, endemic, sporadic, immunodeficiency-associated).

The term “antigen-binding portion” of an antibody (or “antibody portion”) includes fragments of an antibody that retain the ability to specifically hind to an antigen (e.g., a B-cell specific antigen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂, fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P. et al., (1993) Proc. Natl. Acad. Sci. USA 90:6444-6448; Poljak, R. J. et al., (1994) Structure 2:1 I 21-1123). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M. et al., (1995) Human Antibodies and Hybridomas 6:93-101) and use of a cysteine residue, a marker peptide and a C-terminal polyhistidine tag to make bivalent and biotinylated scFv molecules (Kipriyanov, S. M. et al., (1994) Mol. Immunol., 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

“Specific binding,” “specifically hinds,” “specific for”, “selective binding,” and “selectively binds,” as used herein, mean that the compound, e.g., antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). For example, in one embodiment, an antibody or antigen-binding portion thereof, that specifically binds to a B-cell specific antigen, such as, for example, CD-20 or CD-22, will appreciably bind CD-20 or CD-22, but will not significantly react with other non-CD-20 or non-CD-22 proteins or peptides. Specific or selective binding can be determined according to any art-recognized means for determining such binding, including, for example, according to Scatchard analysis and/or competitive binding assays.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The term “human immunoglobulin” or “human antibody” includes antibodies having variable and constant regions corresponding to human germline immunoglobulin sequences as described by Kabat et al. (See Kabat, et al., (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In a preferred embodiment, these replacements are within the CDR regions as described in detail below.

The term “recombinant human antibody” or “recombinant human immunoglobulin” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D. et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat E. A., et al., (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody” includes an antibody that is substantially free of other antibodies having different antigenic specificities (e.g., an isolated antibody that specifically binds a B-cell specific antigen and is substantially free of antibodies or antigen-binding portions thereof that specifically bind other antigens, including other B-cell antigens). An isolated antibody that specifically binds a B-cell specific antigen may bind the same antigen and/or antigen-like molecules from other species. Moreover, an isolated antibody may be substantially free of other cellular material and/or chemicals. Preferably, isolated antibodies are administered to a subject.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The terms “idiotype,” “Id,” and “idiotypic determinant,” as used herein, refer to an epitope in the hypervariable region of an immunoglobulin. Typically, an idiotype or an epitope thereof is formed by the association of the hypervariable or complementarity determining regions (CDRs) of VH and VL domains.

The terms “anti-idiotype” and “anti-Id,” refer to the binding of an antibody or antigen-binding portion thereof to one or more idiotypes.

The term “autologous idiotype vaccine” refers to a composition, the active ingredient of which is an immunogenic molecule that is preferably capable of inducing an immune response against a B-cell idiotype derived from the same subject to which it is administered. In some embodiments, the immunogenic molecule in a vaccine used in the methods of the present invention is a normal product of a subject's B cells that happens to be expressed clonally on the cancer cells (e.g., cells derived from a Hodgkin's lymphoma or non-Hodgkin's lymphoma or chronic lymphocytic leukemia, mantle cell lymphoma or multiple myeloma) and serves as a unique a target for immune attack. In some embodiments, the vaccine comprises an IgM anti-Id immunoglobulin. In some embodiments, an “autologous idiotype vaccine,” is capable of eliciting an immune response against a B-cell idiotype derived from a subject having non-Hodgkin's lymphoma. In another embodiment, an “autologous idiotype vaccine,” is capable of eliciting an immune response against a B-cell idiotype derived from a subject having Hodgkin's lymphoma. In yet another embodiment, an “autologous idiotype vaccine,” is capable of eliciting an immune response against a B-cell idiotype derived from a subject having chronic lymphocytic leukemia. In a further embodiment, an “autologous idiotype vaccine,” is capable of eliciting an immune response against a B-cell idiotype derived from a subject having multiple myeloma. In a yet further embodiment, an “autologous idiotype vaccine,” is capable of eliciting an immune response against a B-cell idiotype derived from a subject having mantle cell lymphoma. In some embodiments of the present invention, an “autologous idiotype vaccine,” is used for the treatment of a B-cell derived cancer in combination with other immune therapeutics such as, for example, monoclonal antibodies that selectively bind B-cell specific antigens. In some embodiments, an “autologous idiotype vaccine” includes an antigen associated with a B-cell derived cancer in a subject (e.g., non-Hodgkin's lymphoma, Hodgkin's lymphoma, chronic lymphocytic leukemia, mantle cell lymphoma or multiple myeloma) linked to KLH (keyhole limpet hemocyanin, a carrier protein). In some embodiments of the present invention, an autologous idiotype vaccine is administered in conjunction with GM-CSF, and subsequently re-administered, as a booster, one or times with or without GM-CSF.

The term “granulocyte monocyte colony stimulating factor” or “GM-CSF” refers to a hematopoeitic growth factor that stimulates the development of committed progenitor cells to neutrophils and enhances the functional activities of neutrophils. It is produced in response to specific stimulation by a variety of cells including macrophages, fibroblasts, endothelial cells and bone marrow stroma. Either purified GM-CSF or recombinant GM-CSF, for example, recombinant human GM-CSF (R & D SYSTEMS, INC, Minneapolis, Minn.) or sargramostim (LEUKINE, BAYER HEALTHCARE Pharmaceuticals, Wayne, N.J.) can be used in the methods described herein.

The phrase “an effective amount of granulocyte monocyte colony stimulating factor” refers to an amount of granulocyte monocyte colony stimulating factor, which upon a single or multiple dose administration to a subject, induces or enhances an immune response in the subject (e.g., as an adjuvant). In some embodiments, 50 μg/m²/day to about 200 μg/m²/day (e.g., 100 μg/m²/day) granulocyte monocyte colony stimulating factor is administered to the subject. In some embodiments, “an effective amount of granulocyte monocyte colony stimulating factor” refers to a daily administration of 5 μg/kg of the granulocyte colony stimulating factor.

As used herein, the terms “treat” or “treatment” refer to both therapeutic treatment and prophylactic or preventative measures, wherein the object is to prevent or slow down (lessen) an undesired physiological change or disorder, such as the development or spread of cancer or other disorder. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. For example, sensitization or tolerization in accordance with the invention can result in therapeutic treatment or prophylaxis of a disorder. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the patient may be identified (e.g., diagnosed) as one suffering from the disease or condition prior to sensitization or tolerization.

As used herein, the term “(therapeutically) effective amount” refers to an amount of an epitope and an IgM Fc region or an IgG Fc region effective to treat a disease or disorder in a mammal (human or non-human mammal). In the case of cancer or other proliferation disorder, the therapeutically effective amount may reduce (i.e., slow to some extent and preferably stop) unwanted cellular proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent administration prevents growth of and/or kills existing cancer cells, it may be cytostatic and/or cytotoxic. For cancer therapy, efficacy can, for example, be measured by assessing the time to disease progression (TTP) and/or determining the response rate (RR). The amount of epitope and IgM Fc region or IgG Fc region may be a growth inhibitory amount.

As used herein, the term “growth inhibitory amount” refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo or in cell culture by greater than about 20%, preferably greater than about 50%, most preferably greater than about 75% (e.g., from about 75% to about 100%).

As used in this specification, the singular forms “a”, “an”, and “the” include plural reference unless the context clearly dictates otherwise. Thus, for example, a reference to “an antibody” means one or more such antibody. A reference to “a molecule” means one or more such molecule, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification Principles and Practice (2nd ed., Springer-Verlag); and PCR: A Practical Approach (McPherson et al. Eds. (1991) IRL Press)), each of which are incorporated herein by reference in their entirety.

Experimental controls are considered fundamental in experiments designed in accordance with the scientific method. It is routine in the art to use experimental controls in scientific experiments to prevent factors other than those being studied from affecting the outcome.

### EXEMPLIFIED EMBODIMENTS

Following are exemplified embodiments of the invention.

### Embodiment 1

A method for directing an immune response to an epitope from an antigen in a subject, comprising:
(a) sensitizing the subject to the epitope, comprising co-administering the epitope and an immunoglobulin M (IgM) constant region (IgM Fc region) to the subject; or
(b) tolerizing the subject to the epitope, comprising co-administering the epitope and an immunoglobulin G (IgG) constant region (IgG Fc region) to the subject.

### Embodiment 2

The method of embodiment 1, wherein the sensitizing of (a) is carried out, wherein the sensitizing of (a) comprises administering a fusion polypeptide comprising the epitope and the IgM Fc region.

### Embodiment 3

The method of embodiment 1, wherein the sensitizing of (a) is carried out, and wherein the sensitizing of (a) comprises administering a nucleic acid molecule encoding the epitope and the IgM Fc region, and wherein the nucleic acid molecule is expressed to produce the epitope and the IgM Fc region separately or as a fusion polypeptide.

### Embodiment 4

The method of embodiment 1, wherein the sensitizing of (a) is carried out, and wherein the sensitizing of (a) comprises co-administering the epitope and the IgM Fc separately, in separate formulations or in the same formulation.

### Embodiment 5

The method of embodiment 1, wherein the sensitizing of (a) is carried out, further comprising administering at least one immune adjuvant (for example, granulocyte-monocyte colony stimulating fragment (GM-CSF) or bovine serum albumin (BSA)) before, simultaneously with, or after co-administration of the epitope and IgM Fc region.

### Embodiment 6

The method of embodiment 1, wherein the sensitizing of (a) is carried out, and wherein the epitope and the IgM Fc region are administered in conjunction with a carrier protein (for example, keyhole limpet hemocyanin (KLH)).

### Embodiment 7

The method of embodiment 1, wherein the tolerizing of (b) is carried out, and wherein the tolerizing of (b) comprises suppression of effector T cell response, suppression of helper T cell response, suppression of B cell response, or suppression of two or more of the foregoing, in the subject.

### Embodiment 8

The method of embodiment 1, wherein the tolerizing of (b) is carried out, and wherein the tolerizing of (b) comprises administering a fusion polypeptide comprising the epitope and the IgG Fc region.

### Embodiment 9

The method of embodiment 1, wherein the tolerizing of (b) is carried out, and wherein the tolerizing of (b) comprises administering a nucleic acid molecule encoding the epitope and the IgG Fc region, and wherein the nucleic acid molecule is expressed to produce the epitope and the IgG Fc region separately or as a fusion polypeptide.

### Embodiment 10

The method of embodiment 1, wherein the tolerizing of (b) is carried out, and wherein the tolerizing of (b) comprises co-administering the epitope and the IgG Fc separately, in separate formulations or in the same formulation.

### Embodiment 11

The method of embodiment 1, wherein the tolerizing of (b) is carried out, further comprising administering a tolerizing agent.

### Embodiment 12

The method of embodiment 11, wherein the tolerizing agent is IVIG (intravenous immunoglobulin IgG) or an immunosuppressant.

### Embodiment 13

The method of embodiment 1, wherein the tolerizing of (b) is carried out on the subject prior to transplantation, and wherein the antigen is an HLA antigen within the donor.

### Embodiment 14

The method of embodiment 1, wherein the subject has cancer, wherein the antigen is a cancer antigen identified in the subject, wherein the sensitizing of (a) is carried out on the subject, wherein the cancer is eliminated or attenuated following the sensitizing of (a), and wherein the tolerizing of (b) is carried out after the cancer is eliminated or attenuated to reduce unwanted autoimmune reaction from the sensitizing of (a).

### Embodiment 15

The method of embodiment 1, wherein the epitope is the epitope of a gene delivery vector, and wherein the tolerizing of (b) is carried out prior to administration of the gene delivery vector to the subject.

### Embodiment 16

The method of embodiment 1, wherein the tolerizing of (b) is carried out on the subject, and wherein the epitope is the epitope of an implant to be introduced into the subject.

### Embodiment 17

The method of embodiment 16, further comprising introducing the implant into the subject after the tolerizing of (b).

### Embodiment 18

The method of embodiment 1, wherein the epitope comprises a mimotope.

### Embodiment 19

The method of embodiment 18, wherein the mimotope is produced by phage display.

### Embodiment 20

The method of embodiment 18, wherein the mimotope is produced by anti-idiotypic antibody generation by immunization of an animal with a monoclonal antibody.

### Embodiment 21

The method of embodiment 1, wherein the antigen is a polypeptide, nucleic acid molecule, carbohydrate, glycoprotein, lipid, lipoprotein, glycolipid, or small molecule.

### Embodiment 22

The method of embodiment 1, wherein the antigen is selected from among a cancer antigen, autoantigen, endogenous antigen, infectious agent antigen, drug (small molecule) antigen, toxin, venom, biologic antigen, environmental antigen (for example, an allergen), transplant antigen, and implant antigen.

### Embodiment 23

The method of embodiment 1, wherein the antigen is a tumor-associated antigen (TAA), and wherein the TAA is a carbohydrate antigen having one or more post-translational modifications that differ from the wild-type protein, comprises a fusion region of a protein resulting from a gene fusion that is present in malignant cells but not present in non-malignant cells, and/or wherein the TAA comprises a receptor tyrosine kinase (RTK) that is deregulated and/or dysfunctional in tumor cells due to autocrine activation, chromosomal translocations, RTK overexpression, or gain-of-function mutations in the RTK gene or protein.

### Embodiment 24

The method of embodiment 1, wherein the antigen is an endogenous antigen, and wherein the endogenous antigen is an aberrantly expressed polypeptide from among amyloid beta, alpha synuclein, cystatin C, tau, ABri, ADan, superoxide dismutase (SOD), mutant Huntington, PrP^{Sc}, or a fragment of any of the foregoing.

### Embodiment 25

The method of embodiment 1, wherein the antigen is an immunoglobulin expressed by a B-cell malignancy.

### Embodiment 26

The method of any one of embodiments 1 to 24, wherein the antigen is not an immunoglobulin.

### Embodiment 27

The method of any one of embodiments 1 to 24, wherein the antigen is not an immunoglobulin expressed by a B-cell malignancy.

### Embodiment 28

The method of embodiment 1, wherein the sensitizing of (a) is carried out, wherein the subject has cancer, and wherein, prior to the sensitizing of (a), the subject undergoes therapy for the cancer (for example, chemotherapy, immunotherapy, radioimmunotherapy, radiation therapy, surgery, or a combination of two or more of the foregoing.

### Embodiment 29

The method of embodiment 28, wherein the cancer is a B-cell malignancy, and wherein the antigen is an immunoglobulin expressed by the B-cell malignancy.

### Embodiment 30

The method of embodiment 1, wherein the sensitizing of (a) is carried out, and wherein the subject has reduced T-regulatory cell activity and/or reduced numbers of T-regulatory cells at the time of co-administration of the epitope and the IgM Fc region.

### Embodiment 31

The method of embodiment 30, wherein the reduced T-regulatory cell activity and/or reduced numbers of T-regulatory cells is induced by administration of a T-regulatory cell inhibitor to the subject.

### Embodiment 32

The method of embodiment 31, wherein the T-regulatory cell inhibitor is selected from among lenalidomide, pomalidomide, an oxazaphosphorine (for example, cyclophosphamide), anti-CD25 monoclonal antibody, IL-2Ra monoclonal antibody, and anti-GITR monoclonal antibody.

### Embodiment 33

The method of any one of embodiments 30-32, wherein the subject has cancer, and wherein the antigen is an antigen of the cancer.

### Embodiment 34

The method of embodiment 33, wherein the cancer is a B-cell malignancy, and wherein the antigen is an immunoglobulin expressed by the B-cell malignancy.

### Embodiment 35

The method of embodiment 25, any one of embodiments 29-32, or embodiment 34, wherein the antigen is an immunoglobulin expressed by a B-cell malignancy, and wherein the immunoglobulin isotype or isotypes exhibited by the malignancy represents an immunoglobulin that is present on the malignant cell (surface), within the malignant cell, secreted by the malignancy or is found in the subject's blood, or any combination of two or more of the foregoing.

### Embodiment 36

The method of embodiment 35, wherein the immunoglobulin isotype or isotypes exhibited by the malignancy is predetermined by obtaining a tumor, tissue or blood sample from the subject by biopsy, needle aspiration, or apheresis.

### Embodiment 37

The method of embodiment 35, wherein the immunoglobulin isotype or isotypes exhibited by the malignancy is predetermined by obtaining a sample of lymph node tissue, extra-nodal tissue, spleen, bone marrow, or blood.

### Embodiment 38

The method of embodiment 35, wherein the immunoglobulin isotype or isotypes exhibited by the malignancy is predetermined by flow cytometry, immunofluorescence, sequencing of heavy chain constant region, or immunoblot.

### Embodiment 39

The method of any preceding embodiment, wherein the subject is human.

### Embodiment 40

A composition comprising an epitope; and an immunoglobulin M (IgM) constant region (IgM Fc region) or an immunoglobulin G (IgG) constant region (IgG Fc region).

### Embodiment 41

The composition of embodiment 40, wherein the composition comprises a fusion polypeptide comprising the epitope and the IgM Fc region.

### Embodiment 42

The composition of embodiment 40, wherein the composition comprises a nucleic acid molecule encoding the epitope and the IgM Fc region, and wherein the nucleic acid molecule is expressed to produce the epitope and the IgM Fc region separately or as a fusion polypeptide.

### Embodiment 43

The composition of embodiment 40, wherein the composition comprises a fusion polypeptide comprising the epitope and the IgG Fc region.

### Embodiment 44

The composition of embodiment 40, wherein the composition comprises a nucleic acid molecule encoding the epitope and the IgG Fc region, and wherein the nucleic acid molecule is expressed to produce the epitope and the IgG Fc region separately or as a fusion polypeptide.

### Embodiment 45

The composition of embodiment 40, wherein the composition comprises the epitope and the IgM Fc region, and wherein the composition further comprises an adjuvant.

### Embodiment 46

The composition of embodiment 40, wherein the composition comprises the epitope and the IgM Fc region, and wherein the composition further comprises a T-regulatory cell inhibitor.

### Embodiment 47

The composition of embodiment 46, wherein the T-regulatory cell inhibitor is selected from among lenalidomide, pomalidomide, an oxazaphosphorine (for example, cyclophosphamide), anti-CD25 monoclonal antibody, IL-2Ra monoclonal antibody, and anti-GITR monoclonal antibody.

### Embodiment 48

The composition of embodiment 40, wherein the composition comprises the epitope and the IgG Fc region, and wherein the composition further comprises an immunosuppressive agent.

### Embodiment 49

The composition of embodiment 40, wherein the composition further comprises an immunomodulatory agent.

### Embodiment 50

The composition of embodiment 40, wherein the epitope is of an antigen that is an immunoglobulin expressed by a B-cell malignancy.

### Embodiment 51

The composition of embodiment 40, wherein the epitope is of an antigen that is not an immunoglobulin.

### Embodiment 52

The composition of embodiment 40, wherein the epitope is of an antigen that is not an immunoglobulin expressed by a B-cell malignancy.

### Embodiment 53

A kit for sensitizing a subject to an epitope of an antigen, wherein the kit comprises at least one IgM Fc region and printed instructions for sensitizing a subject to an epitope using the IgM Fc region.

### Embodiment 54

The sensitizing kit of embodiment 53, further comprising an epitope, adjuvant, carrier protein, an assay for immune response, or any combination of two or more of the foregoing.

### Embodiment 55

The sensitizing kit of embodiment 53 or 54, wherein the kit comprises a fusion polypeptide comprising the epitope and the IgM Fc region.

### Embodiment 56

The sensitizing kit of embodiment 53 or 54, wherein the kit comprises a nucleic acid molecule encoding the epitope and the IgM Fc region, and wherein the nucleic acid molecule is expressed to produce the epitope and the IgM Fc region separately or as a fusion polypeptide.

### Embodiment 57

The sensitizing kit of any one of embodiments 53-56, further comprising a T-regulatory cell inhibitor.

### Embodiment 58

The sensitizing kit of embodiment 57, wherein the T-regulatory cell inhibitor is selected from among lenalidomide, pomalidomide, an oxazaphosphorine (for example, cyclophosphamide), anti-CD25 monoclonal antibody, IL-2Ra monoclonal antibody, and anti-GITR monoclonal antibody.

### Embodiment 59

The sensitizing kit of any one of embodiments 53 to 58, wherein the epitope is of an antigen that is an immunoglobulin expressed by a B-cell malignancy.

### Embodiment 60

The sensitizing kit of any one of embodiments 53 to 58, wherein the epitope is of an antigen that is not an immunoglobulin.

### Embodiment 61

The sensitizing kit of any one of embodiments 53 to 58, wherein the epitope is of an antigen that is not an immunoglobulin expressed by a B-cell malignancy.

### Embodiment 62

A kit for tolerizing a subject to an epitope, wherein the kit comprises at least one IgG Fc region and printed instructions for tolerizing a subject to an epitope.

### Embodiment 63

The tolerizing kit of embodiment 62, further comprising an epitope, adjuvant, carrier protein, an assay for T-regulatory cell number and/or activity, an assay for immune response, or any combination of two or more of the foregoing.

### Embodiment 64

The tolerizing kit of embodiment 62 or 63, wherein the kit comprises a fusion polypeptide comprising the epitope and the IgG Fc region.

### Embodiment 65

The tolerizing kit of embodiment 62 or 63, wherein the kit comprises a nucleic acid molecule encoding the epitope and the IgG Fc region, and wherein the nucleic acid molecule is expressed to produce the epitope and the IgG Fc region separately or as a fusion polypeptide.

### Embodiment 66

The tolerizing kit of any one of embodiments 62 to 65, wherein the epitope is of an antigen that is an immunoglobulin expressed by a B-cell malignancy.

### Embodiment 67

The tolerizing kit of any one of embodiments 62 to 65, wherein the epitope is of an antigen that is not an immunoglobulin.

### Embodiment 68

The tolerizing kit of any one of embodiments 62 to 65, wherein the epitope is of an antigen that is not an immunoglobulin expressed by a B-cell malignancy.

### Embodiment 69

A kit for sensitizing or tolerizing a subject to an epitope, wherein the kit comprises at least one IgM Fc region, at least one IgG Fc region, printed instructions for sensitizing a subject to an epitope using the IgM Fc region, and printed instructions for tolerizing a subject to an epitope using the IgM Fc region.

### Embodiment 70

The sensitizing/tolerizing kit of embodiment 69, wherein the sensitizing/tolerizing kit further comprises an epitope, adjuvant, carrier protein, or any combination of two or more of the foregoing.

### Embodiment 71

The sensitizing/tolerizing kit of embodiment 69 or 70, wherein the kit comprises a fusion polypeptide comprising the epitope and the IgM Fc region; a fusion polypeptide comprising the epitope and the IgG Fc region, or both.

### Embodiment 72

The sensitizing/tolerizing kit of embodiment 69 or 70, wherein the kit comprises (a) a nucleic acid molecule encoding the epitope and the IgM Fc region, wherein the nucleic acid molecule is expressed to produce the epitope and the IgM Fc region separately or as a fusion polypeptide; (b) a nucleic acid molecule encoding the epitope and the IgG Fc region, wherein the nucleic acid molecule is expressed to produce the epitope and the IgG Fc region separately or as a fusion polypeptide; or both (a) and (b).

### Embodiment 73

The sensitizing/tolerizing kit of any one of embodiments 69 to 72, further comprising a T-regulatory cell inhibitor.

### Embodiment 74

The sensitizing/tolerizing kit of embodiment 73, wherein the T-regulatory cell inhibitor is selected from among lenalidomide, pomalidomide, an oxazaphosphorine (for example, cyclophosphamide), anti-CD25 monoclonal antibody, IL-2Ra monoclonal antibody, and anti-GITR monoclonal antibody.

### Embodiment 75

The sensitizing/tolerizing kit of any one of embodiments 69 to 74,

wherein the epitope is of an antigen that is an immunoglobulin expressed by a B-cell malignancy.

### Embodiment 76

The sensitizing/tolerizing kit of any one of embodiments 69 to 74,

wherein the epitope is of an antigen that is not an immunoglobulin.

### Embodiment 77

The sensitizing/tolerizing kit of any one of embodiments 69 to 74,

wherein the epitope is of an antigen that is not an immunoglobulin expressed by a B-cell malignancy.

### Embodiment 78

A kit for detecting the T-regulatory (T-reg) cell response before, during, and/or after administration of a T-reg inhibitor prior to administration of an epitope and an immunoglobulin M (IgM) constant region (IgM Fc region), wherein the kit comprises one or more reagents for assessing T-reg cell response in a subject; and printed instructions for making the assessment.

### Embodiment 79

The kit of embodiment 78, further comprising a T-regulatory cell inhibitor.

### Embodiment 80

The kit of embodiment 79, wherein said T-regulatory cell inhibitor is selected from among lenalidomide, pomalidomide, an oxazaphosphorine (for example, cyclophosphamide), anti-CD25 monoclonal antibody, IL-2Ra monoclonal antibody, and anti-GITR monoclonal antibody

### Embodiment 81

The kit of any one of embodiments 78 to 80, further comprising the epitope, or the IgM Fc region, or both.

### Embodiment 82

The kit of embodiment 81, wherein the kit comprises a fusion polypeptide comprising the epitope and the IgM Fc region.

### Embodiment 83

The kit of embodiment 81, wherein the kit comprises a nucleic acid molecule encoding the epitope and the IgM Fc region, and wherein the nucleic acid molecule is expressed to produce the epitope and the IgM Fc region separately or as a fusion polypeptide.

### Embodiment 84

The kit of any one of embodiments 78 to 83, wherein the epitope is of an antigen that is an immunoglobulin expressed by a B-cell malignancy.

### Embodiment 85

The kit of any one of embodiments 78 to 83, wherein the epitope is of an antigen that is not an immunoglobulin.

### Embodiment 86

The kit of any one of embodiments 78 to 83, wherein the epitope is of an antigen that is not an immunoglobulin expressed by a B-cell malignancy.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Materials and Methods

Patients.

Written informed consent was obtained from patients prior to study entry. Eligible patients had a diagnosis of FL, grade 1, 2, or 3a, confirmed by central pathology review (EST); had monoclonal surface IgM or IgG on tumor; were chemotherapy naïve; had bulky (>5 cm) stage II, stage III or IV disease with a lymph node >2 cm accessible for biopsy.

Study Design.

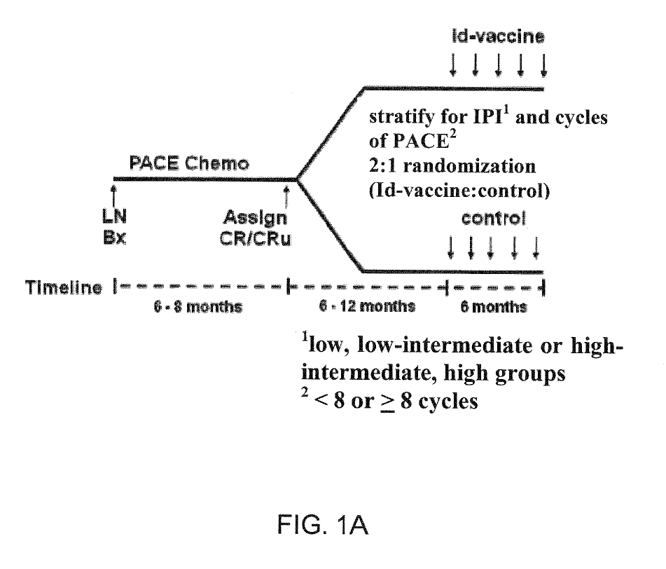

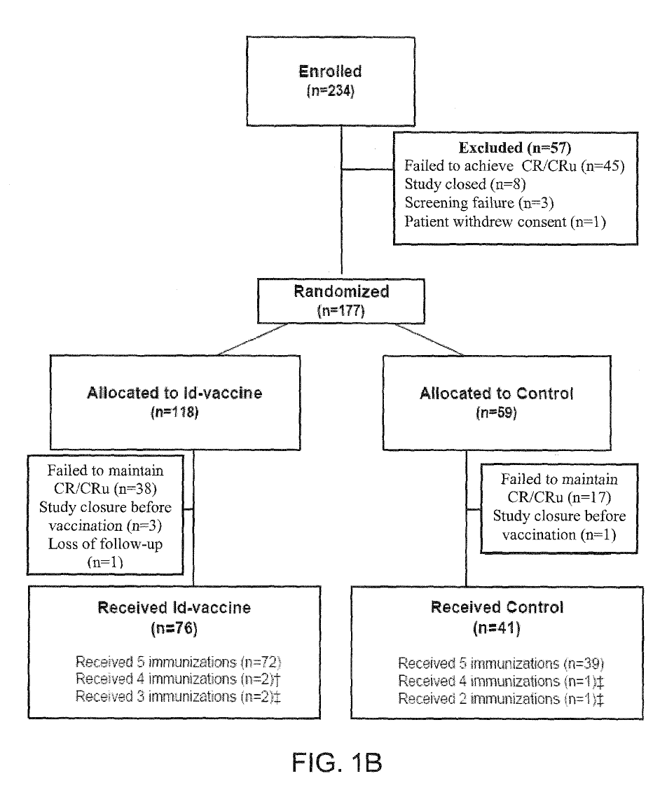

This prospective randomized double-blind controlled trial was initiated in January 2000 at the NCI and subsequently expanded to 17 centers in the USA and Europe under sponsorship by Biovest International after institutional review board approval at each center. All patients underwent an excisional lymph node biopsy to confirm pathology, and to provide material for Id protein production (FIG. 1A). Patients who achieved complete response (CR)/CR unconfirmed (CRu)¹³ after PACE chemotherapyl^{12,14,15} were stratified by International Prognostic Index (IPI) Risk Group (0-2 vs. 3, 4)¹⁶ and number of chemotherapy cycles (=8 vs. >8) and randomized 2:1 to receive either Id-vaccine (Id-KLH+GM-CSF) or control (KLH+GM-CSF). Randomization was performed centrally through a concealed web-based random allocation system by EMMES Corporation, Rockville, Md. Patients with less than CR/CRu after chemotherapy were excluded from randomization. The protocol was amended in 2007 to allow CHOP-R as induction therapy.

Vaccine Therapy.

Tumor isotype-matched Id protein was manufactured by heterohybridoma technology.^{12,18}At study initiation, the estimated time for Id-vaccine production was 6-12 months. To ensure that the physicians and patients remained blinded to the treatment, the release dates for the Id-vaccine and control were matched using an algorithm. Depending on the release dates, randomized patients who remained in CR/CRu, received 5 blinded Id-vaccine or control injections at 1, 2, 3, 4 and 6 months starting between 6-12 months after completion of chemotherapy. Patients received isotype-matched (IgM or IgG) Id-KLH or KLH 0.5 mg each subcutaneously on day 1 with GMCSF 100 μg/m²/day subcutaneously on days 1-4. Patients randomized to receive Id-vaccine for whom Id protein could not be made received KLH+GM-CSF but were analyzed as randomized.

Study Evaluation.

Physical examination; computed tomography (CT) scans of chest, abdomen, and pelvis; and bone marrow examination were performed prior to chemotherapy, after cycle four and every two cycles thereafter, prior to first vaccination, and 4 weeks after fifth vaccination. Thereafter, physical examination and CT scans were performed every 6 months until relapse. Tumor response was assessed by study investigators blinded to treatment assignment according to the International Workshop response criteria for NHL.¹³ The Common Toxicity Criteria version 2.0 was used for adverse event (AE) reporting.

Statistical Analysis.

The primary objective of the study was to determine whether Id vaccination prolonged DFS compared to control in FL patients in durable CR/CRu after chemotherapy. Two prospective efficacy analyses were performed to compare DFS between treatment arms: 1) all randomized patients and 2) randomized patients remaining in CR/CRu at the time of vaccination and receiving at least one blinded vaccination. Secondary objectives were to evaluate the safety, to compare OS between the treatment aims, and to evaluate immunologic and molecular responses. The study intended to enroll 563 patients and 375 were expected to attain CR/CRu. Of the 375 patients, 250 would be randomized to receive Id-vaccine and 125 to receive control. This number is sufficient to allow approximately 80% power to detect a 50% reduction in hazard in the experimental arm with minimum follow-up of 8 months. DFS was calculated from date of randomization until date of relapse or last follow-up. Overall survival was calculated from date of randomization until death or last follow-up. Kaplan-Meier survival curves were constructed and the log-rank statistic used to test statistical differences using SAS. The trial was monitored annually by an independent Data Monitoring Committee (DMC). Until the time 50 patients were randomized, only toxicity was examined at annual review. Once 50 patients were randomized, annual interim evaluations were performed to determine whether there was sufficient evidence to terminate accrual because of a better than expected improvement in DFS. Interim outcome results were blinded to the trial investigators. All patients were followed for as long as possible to obtain survival information.

In an unplanned exploratory analysis, the inventors compared DFS of Id-vaccinated patients with control patients separately depending on tumor Ig isotype. To address whether there was a differential treatment effect on DFS depending on Ig isotype, the inventors used Cox proportional hazards modeling; in addition to both as main effects, the inventors included an interaction term between treatment and Ig isotype and IPI and number of chemotherapy cycles as covariates.

Chemotherapy.

PACE chemotherapy was administered as follows: cyclophosphamide 650 mg/m2 IV, doxorubicin 25 mg/m2 IV, and etoposide 120 mg/m2 IV on days 1 and 8, and prednisone 60 mg/m2 orally daily for 14 days (days 1 to 14) of a 28 day treatment cycle. Prophylactic sulfamethoxazole (800 mg) and trimethoprim (160 mg) orally three times per week was used during chemotherapy. The doses of cyclophosphamide, doxorubicin, and etoposide could be increased by 10% for the second and subsequent cycles if the granulocyte nadir on day 22 of the previous cycle was >750/μl. Dose escalations were instituted at the discretion of the treating physician and were not mandatory. Patients achieving a CR/CRu after four or six cycles of PACE received two additional cycles of therapy. Patients achieving a CR/CRu after eight cycles stopped therapy and were randomized without receiving additional therapy. Patients who had not attained a CR/CRu after eight cycles, but whose disease was continuing to respond to therapy, could receive additional cycles of chemotherapy without doxorubicin until CR/CRu. Patients whose disease was stable for two cycles of chemotherapy without attaining CR/CRu or who developed progressive disease were removed from the study and not randomized. A minimum of six cycles of PACE was given to each complete responder before chemotherapy was discontinued.

Vaccine Production.

Idiotype-protein was manufactured by heterohybridoma technology. Briefly, lymphoma tumor cells obtained from the lymph node biopsy from each patient were fused to hypoxanthineaminopterin-thymidine-sensitive heterohybridoma K6H6/B5 cells to produce hybridomas preserving the tumor Ig isotype (identified following biopsy by flow cytometry or immunohistochemistry). Hybridomas secreting the tumor idiotype were identified by comparing the immunoglobulin heavy chain CDR3 sequences of the fusions with the patient's tumor. Selected hybridoma clones matching the tumor Ig isotype (IgM or IgG) were expanded and the protein was purified from the culture supernatant by affinity chromatography using 1D12 anti-IgM antibody columns for IgM purification and Protein A column for IgG purification. Purified isotype-matched Id protein (IgM-Id or IgG-Id) was conjugated to KLH using glutaraldehyde.

Algorithm for Vaccine Release.

The assumption at the initiation of the protocol was that the average production time for Id-KLH+GM-CSF vaccine (Id-vaccine) would be approximately eight months, with 50% released between the 6.5 and 9.5 months, and the remaining times would be distributed approximately evenly between 6.0 and 6.5 months (25%), or between 9.5 and 12.0 months (25%). Modifications to release dates based on changes in distribution were performed as needed. The algorithm for the KLH+GM-CSF (control) release times on the control arm was generated according to the following overall distribution: 25% are category 1 (6.0-6.5 months=183-197 days); 50% are category 2 (6.5-9.5 months=198-288 days); 25% are category 3 (9.5-12 months=289-365 days). The algorithm for the initial set of release times was as follows: (1) within each stratum, the control release times were generated in blocks of 24; (2) within each block of 24 control patients, the control release time categories were randomly assigned in such a way that categories one and three had 6 patients each, and category two had 12 patients; (3) within each category, the actual time of release was assigned from a uniform distribution over the length of the category, for example, within category one which spans 183-197 days, each patient had a 1/15 chance of being assigned to each of those days. Since the overall distribution of release times was subject to modification as the trial continued, the actual distribution of the initial 48 Id-vaccine release times was compared to that of the first 24 control release times in order to validate that the two distributions were close to one another. If the first 24 Id-vaccine release times had a distribution which did not reflect the actual control times from the first 48 Id patients (different median, different amount of spread in values, etc.) that information was used, focusing on the most recent 24 Id-vaccine release times, to provide a different set of release times for the subsequent 24 control patients. This process was repeated throughout the trial so that each block of 24 control release times reflected the distribution of the prior 48 Id-vaccine release times. If a change in process occurred at any time which would warrant that a more rapid adjustment in the control release times take place, this took precedence over a planned evaluation. The most important aspect of this process was that the physicians involved in direct patient evaluation should be unable to discern which agent was being administered on the basis of the release date.

Sample Size Calculation.

The following parameters were used to estimate the sample size of the study: (1) ⅔ of patients entered onto the trial were expected to achieve CR/CRu from PACE chemotherapy; (2) the median DFS for patients with FL treated with PACE chemotherapy alone was expected to be 3.5 years; and (3) it was expected that 15% of patients who would be randomized to receive Id-vaccine will not be able to have a vaccine made for them. Sample size calculations were performed based on simulations assuming an intent-to-treat analysis, equal hazards (1.0 hazard ratio) for the first 8 months (when treatments are expected to be the same in both randomized arms), and then a hazard ratio of 2.0 after 8 months. A two-sided hypothesis test at the alpha=0.01 level was used to ensure a stringent evaluation. Accrual was estimated to take 5 years with follow-up for 4 additional years after the last patient was entered. A 2:1 randomization favoring Id-vaccine was used to gain the most information about the effects of vaccine in this group of patients.

Analysis of DFS by Isotype.

For analysis of DFS by tumor Ig heavy-chain isotype, the inventors grouped patients according to their vaccine isotype (IgM or IgG) if an Id-vaccine was successfully manufactured (see Table 6). Patients for whom a vaccine could not be manufactured were analyzed according to their biopsy isotype if the isotype was homogeneous in the biopsy (either IgM or IgG). Patients for whom the biopsy isotype was heterogeneous with mixed IgM/IgD or IgM/IgA were assigned to the IgM group, and patients for whom the biopsy isotype was heterogeneous with mixed IgG/IgA were assigned to the IgG group. Patients for whom the biopsy isotype was heterogeneous with mixed IgM/IgG isotype and/or did not receive any Id-vaccine were excluded from the analysis.

### Example 1

### Vaccination with Patient-Specific Hybridoma-Derived ID Protein Vaccine Prolongs Disease-Free Survival in Follicular Lymphoma Patients

Starting in January 2000, a total of 234 patients were enrolled in the study (FIG. 1B and Table 4). Due to protracted enrollment (Table 5), the trial was terminated prior to full accrual and the data were locked on Jun. 30, 2008, following DMC recommendation. At study termination, 219 patients completed PACE chemotherapy and 6 completed cyclophosphamide, doxorubicin, vincristine, prednisone, and rituximab (CHOP-R) chemotherapy. Of the patients who received PACE, 177 (81%) achieved CR/CRu, and were stratified and randomized to receive either Id-vaccine (n=118) or control (n=59) (Table 4). Fifty-seven (24%) patients were excluded from randomization because of failing to achieve CR/CRu (n=45), study closure (n=8), screening failure (n=3), or withdrawing consent (n=1). Patients who received CHOP-R were among the 57 patients excluded either due to study closure (n=3) or for failing to achieve CR/CRu (n=3). Prior to vaccination, 55 (31%) randomized patients relapsed (38 in the Id-vaccine arm, 17 in the control arm); and 5 randomized patients were excluded due to study closure (3 in the Id-vaccine arm, 1 in the control arm) or loss of follow-up (1 in the Id-vaccine arm). Of the 117 patients who received at least one blinded vaccination, 76 received Id-vaccine and 41 received control. As expected from the vaccine release algorithm, the median time between randomization and initiation of vaccinations was not significantly different between the Id-vaccine (8.74 months) and control (8.31 months) arms (P=0.279). Idiotype protein was successfully produced in 72 of 76 patients (95%) assigned to receive the Id-vaccine. Five patients assigned to the experimental arm received KLH+GM-CSF due to failure to make Id protein but were analyzed as randomized. Six patients did not complete the five intended vaccinations either due to withdrawal (n=2) or relapsed disease (n=4) but were analyzed as randomized. All baseline characteristics were well balanced between the groups that received blinded vaccinations (n=117) (Table 1) as well as between the two groups of the 60 randomized patients that did not receive vaccinations (Table 2).

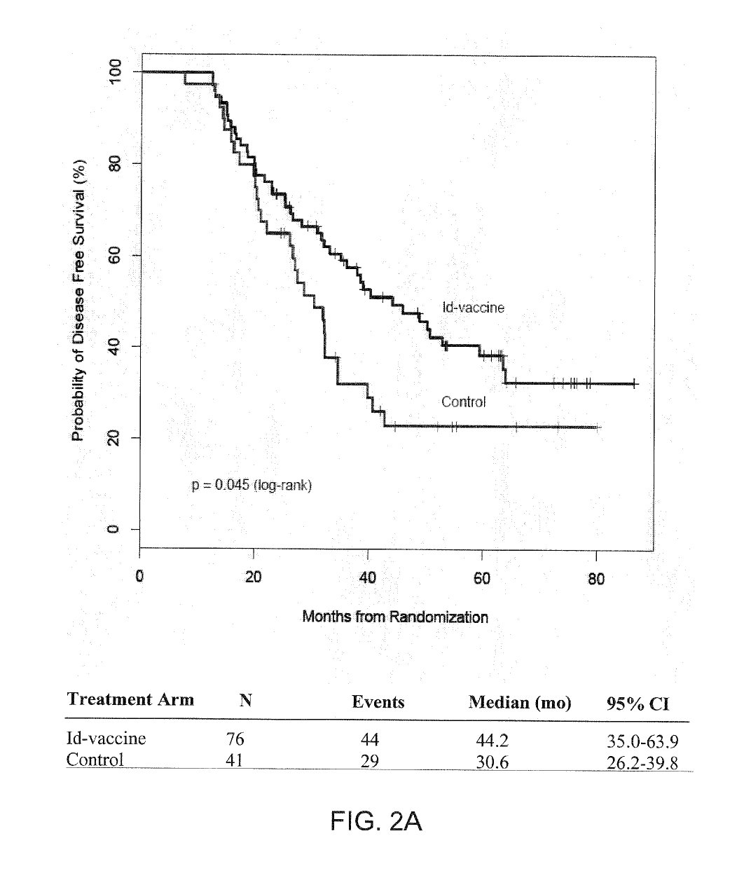

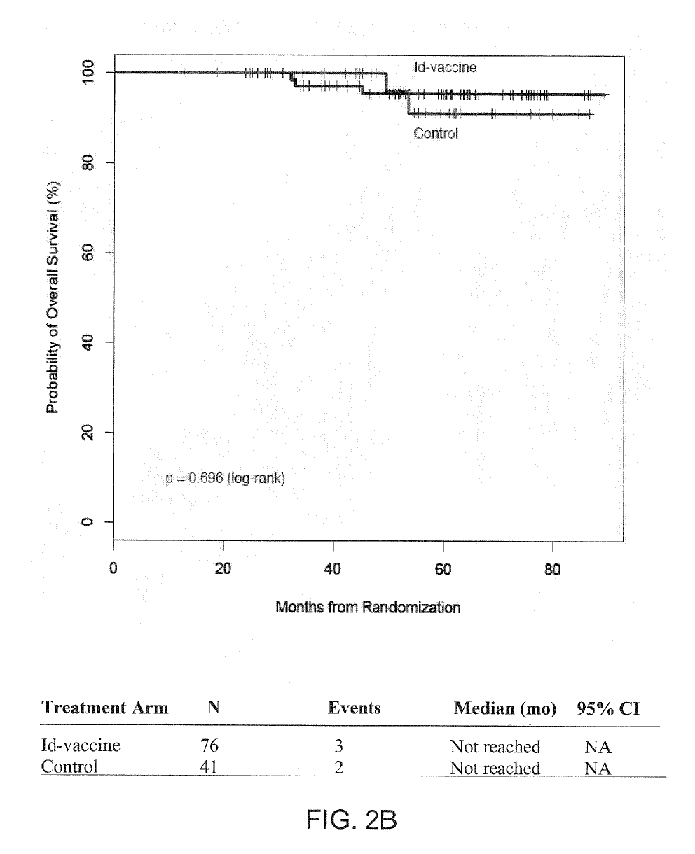

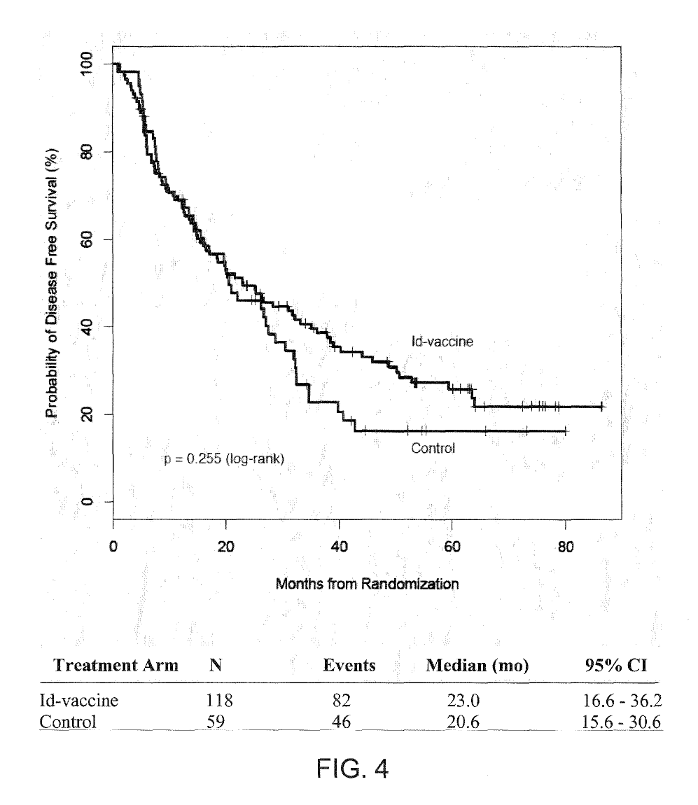

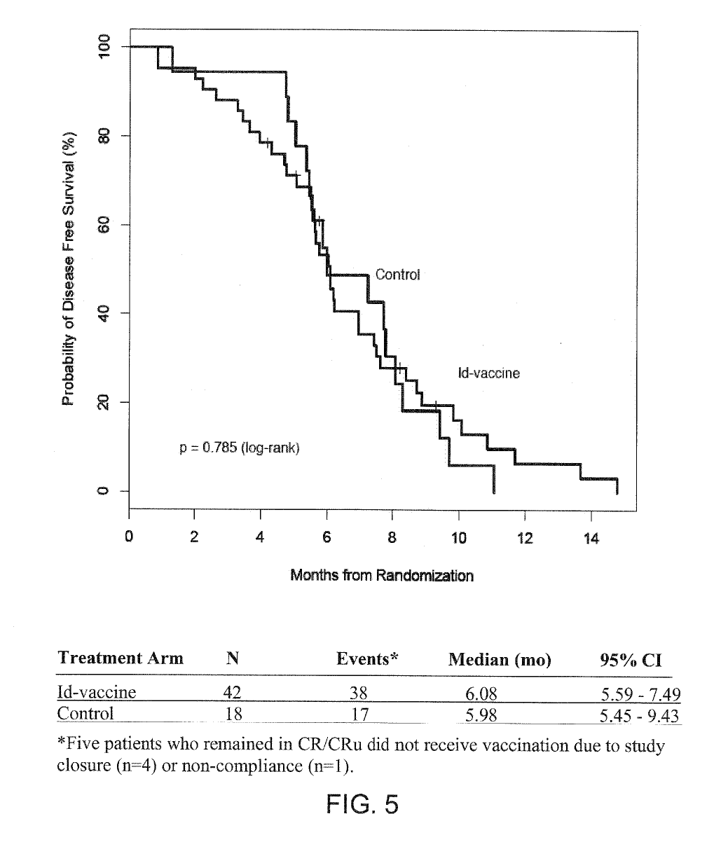

For the 117 patients who received at least one blinded vaccination, median DFS was significantly prolonged in the Id-vaccine arm compared to the control arm (FIG. 2A). At a median follow-up of 56.6 months (range 12.6-89.3 months), median DFS after randomization to the Id-vaccine arm was 44.2 months versus 30.6 months for the control arm (P=0.045). Using Cox proportional hazard model, a statistically-significant hazard ratio (HR) of 0.62 was achieved (P=0.047; 95% confidence interval [CI]: 0.39-0.99). Median overall survival (OS) was not reached for either group; the number of deaths were too few to enable any conclusions about overall survival (FIG. 2B). For all 177 randomized patients, median DFS from randomization between the Id-vaccine and control arms was 23.0 vs. 20.6 months, respectively (P=0.256; HR=0.81; 95% CI: 0.56-1.16) (FIG. 4). There was no statistically-significant difference in median DFS between arms for the 60 randomized patients who did not receive vaccinations (6.08 months for Id-vaccine arm vs. 5.98 months for control arm; P=0.78; HR=0.92; 95% CI: 0.51-1.65) (FIG. 5) suggesting that the arms were well balanced for baseline characteristics (Table 2). Analysis of the group of 117 patients who received at least one blinded vaccination showed statistically-significant improvement in DFS in the Id-vaccine arm compared to the control arm (FIG. 2A).

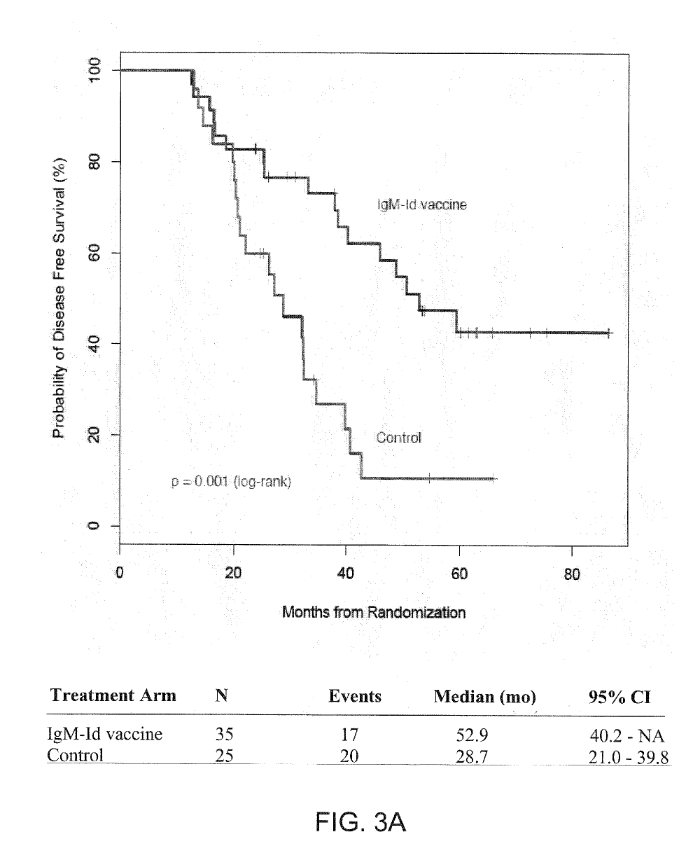

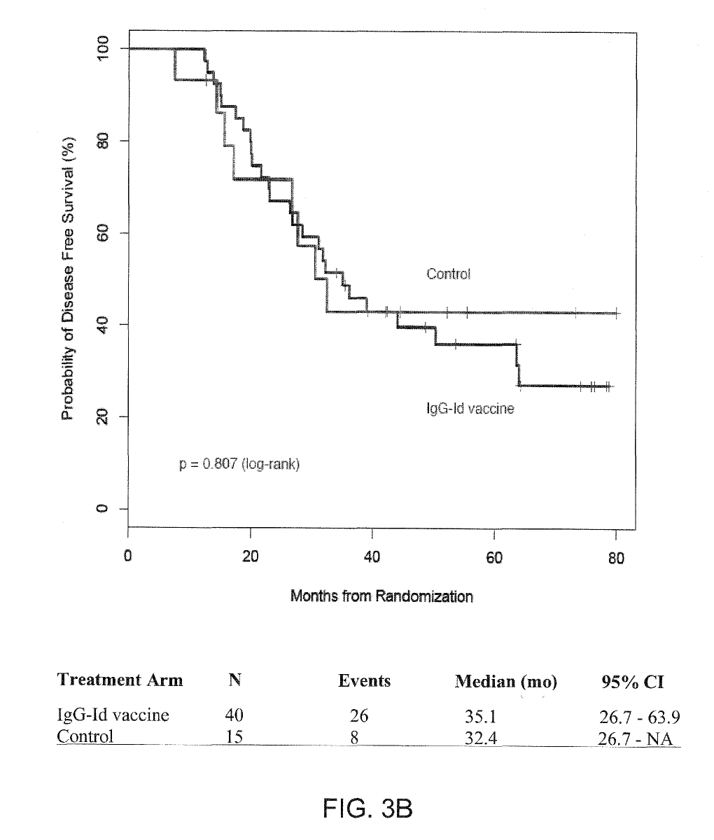

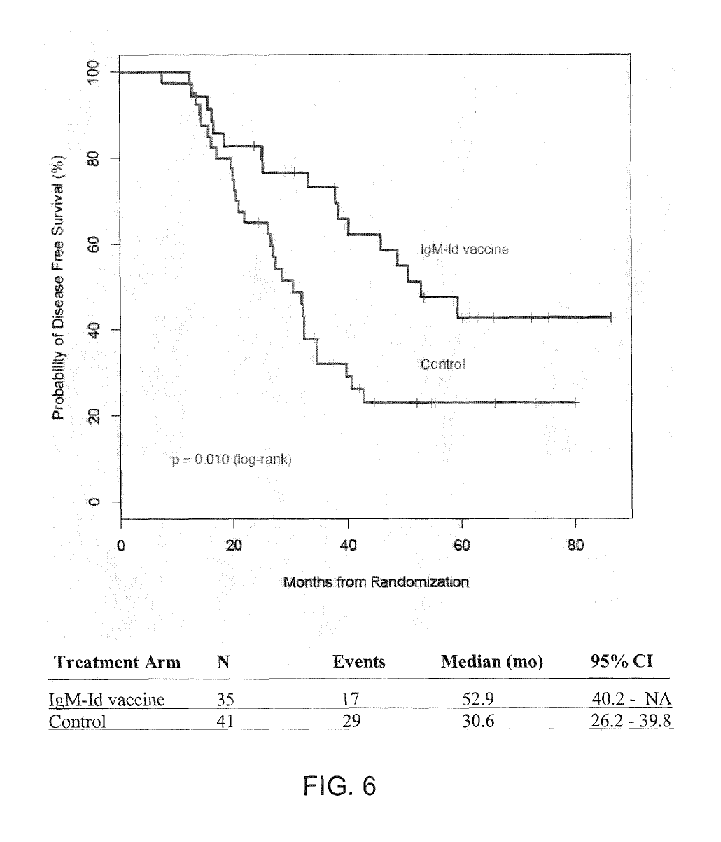

DFS of vaccinated patients was also analyzed by tumor Ig heavy- and light-chain isotypes. For IgM and IgG heavy-chain isotype groups, there were no statistically significant differences in baseline patient characteristics between experimental and control arms (n=35 vs. 25 for IgM isotype and n=40 vs. 15 for IgG isotype for Id-vaccine and control arms, respectively). Two patients had mixed IgM/IgG biopsy isotypes and were excluded from this analysis (Tables 4 and 6). Among patients receiving an IgM-Id vaccine, median time to relapse after randomization was 52.9 months, versus 28.7 months in the IgM tumor isotype control-treated patients (p=0.001; HR=0.34 [p=0.002]; 95% CI:0.17-0.68) (FIG. 3A) and 30.6 months in all controls (p=0.010; FIG. 6). Among patients receiving an IgG-Id vaccine, median time to relapse after randomization was 35.1 months, versus 32.4 months in the IgG tumor isotype control-treated patients (p=0.807; HR=1.1 [p=0.807]; 95% CI:0.50-2.44) (FIG. 3B). Cox proportional hazard modeling supports an interaction between treatment and Ig isotype (p=0.039). When patients were grouped by light chain type, there was no difference in DFS (data not shown).

Both Id-vaccine and control were safe and well-tolerated. There were no statistically significant differences in frequency or types of AE observed between groups. Grade 1-2 AE were common in both groups (Table 7). However, grade 3-4 AE were rare; there were no Id-vaccine-related deaths (Table 3). The most common AE were injection site reactions (>80% of patients on each arm) with erythema and induration lasting for a few days after each vaccination.

This controlled clinical trial demonstrates that vaccination with patient-specific hybridoma-derived Id protein vaccine prolongs DFS, compared to controls, in FL patients vaccinated during a CR/CRu lasting at least six months after PACE chemotherapy. The principal focus of the efficacy analysis was on the group of patients receiving at least one blinded vaccination. For this patient group, the results showed a statistically significant improvement in DFS following Id vaccination, compared with the control arm (FIG. 2A). In general, the ideal time for randomization is at the time of initiating experimental therapy. However, the decision was made the decision to randomize well in advance, immediately after completion of chemotherapy, so that resources would not be expended manufacturing patient-specific vaccines for the control group. Nevertheless, the conclusions should have the same validity as if randomization had occurred at initial vaccination, with the principal potential concern that patients in one arm may be more likely to drop out of the study before vaccination. Indeed, DFS analysis of the 60 patients who were randomized but not vaccinated showed no suggestion of treatment effect (FIG. 5), demonstrating that the arms were well balanced for baseline characteristics (Table 2). Furthermore, the concealed randomization, the double-blinded nature of the study, the use of a vaccine release algorithm to achieve comparable time from randomization to vaccination, the similar rate of injection site reactions in both groups (Table 3), and the analysis of data by an independent statistician guarded against the introduction of unintentional bias in the efficacy analysis of the 117 vaccinated patients. The improvement in DFS with the Id-vaccine (FIG. 2A) despite the use of KLH+GM-CSF, a potentially active form of immunotherapy,^{19,20} in the control also suggests that the clinical benefit induced by the Id-vaccine may have been even greater had the control group received a placebo. The treatment comparison for all 177 randomized 15 patients was not statistically significant (FIG. 4) because inclusion of the 60 non-vaccinated patients obscured the treatment effect shown in FIG. 2A.

Although termination of the trial before completion of the planned accrual resulted in a smaller sample size than originally intended and decreased the power to detect a difference in DFS between treatment arms, the study, nevertheless, showed a statistically significant improvement in DFS for Id vaccinated patients (FIG. 2A). As previously suggested, randomized trials may overcome limitations of small sample size and yield valid conclusions if baseline characteristics are well balanced, allocation is concealed, and they are double-blinded.^{21,22} These features, built into this trial, together with the fact that the HR for DFS is 0.62 (FIG. 2A), support the conclusion that the treatment effect observed by this vaccine was not exaggerated.

To determine whether isotype of the surface immunoglobulin used as an idiotype vaccine influences clinical response, DFS of vaccinated patients was analyzed according to their tumor Ig isotype. The inventors observed that patients immunized with IgM-Id vaccines had significantly longer DFS than control patients with IgM isotype tumors, while DFS for those receiving IgG-Id vaccines did not differ from isotype-matched controls (FIG. 3). Although this trial was not powered to address such subset analysis and this analysis was not pre-specified in the protocol, the observed treatment effects differ dramatically by isotype. While the epitopes after Id vaccination have been shown to be derived from the unique variable region of the tumor's immunoglobulin,^{25,26} the isotype of the constant region may influence the immunogenicity of variable region epitopes.^{23,24} Preclinical studies have shown that Ids that have switched to IgG became tolerogenic, while Ids of their IgM progenitors were highly immunogenic.^{23,24} The improvement in DFS observed for patients receiving Id-vaccine in this trial stands in contrast to the results of the Genitope²⁷ and Favrille²⁸ phase III trials, which failed to show clinical benefit with recombinant tumor-derived Id vaccines in FL. The significant differences in trial design and vaccine formulation are likely responsible for the different clinical outcomes observed in these three phase 3 trials (Table 8). The present study used the phase II NCI treatment protocol and the hybridoma Id protein manufacturing method.^{12,18} With regard to trial designs, the Favrille and Genitope trials differed significantly from this trial by extending eligibility to patients with partial response and stable disease in addition to CR/Cru after chemotherapy, using less aggressive induction chemotherapy prior to vaccination, and not stratifying by clinical prognostic factors for treatment allocation. It is conceivable that the benefit of Id vaccination is discernable only in patients with minimal residual disease (CR/CRu) after chemotherapy. The hybridoma technique¹⁸ used in this trial yields Id proteins that more closely resembled the native Ig on the tumor cell surface, compared with the recombinant DNA-derived Id proteins used in the Genitope and Favrille studies.¹⁰ Production of recombinant protein may have altered post-translational modifications such as glycosylation, which can result in profound changes in final protein tertiary structure.²⁹ In addition, the hybridoma technique yields Id proteins with IgM or IgG Fc regions identical to the tumor Ig isotype as opposed to a universal IgG Fc used to produce Id vaccines for all patients in the Genitope and Favrille trials. It is possible that the use of a universal IgG Fc may have altered the immunogenicity of the Id vaccine (FIG. 3). This trial was initiated in the pre-rituximab era and used standard combination chemotherapy as the induction regimen. In current practice, chemotherapy is administered with rituximab, an anti-CD20 monoclonal antibody shown to improve overall response rate, progression-free survival, and overall survival in FL patients.^{2,3,30,31} However, rituximab-containing immunochemotherapies do not appear to be curative and complementary treatment strategies are needed.^{30,31} Although rituximab induces prolonged B-cell deletion and impairs induction of humoral responses following Id vaccination, generation of tumor-specific cellular immunity is not affected.³² Phase I and II clinical trials suggest that tumor-specific humoral and cellular immune responses after Id vaccination may each independently induce tumor regression and have been associated with improvement in clinical outcome in FL.^{10-12,33,34} While the relative importance of humoral versus cellular immunity in the efficacy of Id vaccination is unclear, cellular immunity induced by Id vaccination could, conceptually, complement rituximab-containing immunochemotherapies.^{2,5}

This trial proves the principle that therapeutic vaccination can result in meaningful clinical benefit for FL patients by prolonging DFS. Furthermore, the results of this trial suggest that the isotype of the constant region may influence the immunogenicity of Id vaccines. This finding could have profound implications on Id vaccine production strategies and clinical development for FL and other B-cell malignancies.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. A clinical evaluation of the International Lymphoma Study Group classification of non-Hodgkin's lymphoma. The Non-Hodgkin's Lymphoma Classification Project. Blood 89:3909-18 (1997).
2. Hiddemann W, Kneba M, Dreyling M, et al: Frontline therapy with rituximab added to the combination of cyclophosphamide, doxorubicin, vincristine, and prednisone (CHOP) significantly improves the outcome for patients with advanced-stage follicular lymphoma compared with therapy with CHOP alone: results of a prospective randomized study of the German Low-Grade Lymphoma Study Group. Blood 106:3725-32 (2005).
3. Marcus R, Imrie K, Solal-Celigny P, et al: Phase III study of R-CVP compared with cyclophosphamide, vincristine, and prednisone alone in patients with previously untreated advanced follicular lymphoma. J Clin Oncol 26:4579-86 (2008).
4. Stevenson, G. T. & Stevenson, F. K. Antibody to a molecularly-defined antigen confined to a tumour cell surface. Nature 254, 714-716 (1975).
5. Sirisinha, S. & Eisen, H. N. Autoimmune-like antibodies to the ligand-binding sites of myeloma proteins. Proc Natl Acad Sci USA 68, 3130-3135 (1971).
6. Lynch, R. G., Graff, R. J., Sirisinha, S., Simms, E. S. & Eisen, H. N. Myeloma proteins as tumor-specific transplantation antigens. Proc Natl Acad Sci USA 69, 1540-1544 (1972).
7. Kaminski, M. S., Kitamura, K., Maloney, D. G. & Levy, R. Idiotype vaccination against murine B cell lymphoma. Inhibition of tumor immunity by free idiotype protein. J Immunol 138, 1289-1296 (1987).
8. Kwak, L. W., Young, H. A., Pennington, R. W. & Weeks, S. D. Vaccination with syngeneic, lymphoma-derived immunoglobulin idiotype combined with granulocyte/macrophage colony-stimulating factor primes mice for a protective T-cell response. Proc Natl Acad Sci USA 93, 10972-10977 (1996).
9. Kwak, L. W., et al. Induction of immune responses in patients with B-cell lymphoma against the surface-immunoglobulin idiotype expressed by their tumors. N Engl J Med 327, 1209-1215 (1992).
10. Park, H. J. & Neelapu, S. S. Developing idiotype vaccines for lymphoma: from preclinical studies to phase III clinical trials. Br J Haematol 142, 179-191 (2008).
11. Houot, R. & Levy, R. Vaccines for lymphomas: idiotype vaccines and beyond. Blood Rev 23, 137-142 (2009).
12. Bendandi, M., et al. Complete molecular remissions induced by patient-specific vaccination plus granulocyte-monocyte colony-stimulating factor against lymphoma. Nat Med 5, 1171-1177 (1999).
13. Cheson B D, Horning S J, Coiffier B, et al: Report of an international workshop to standardize response criteria for non-Hodgkin's lymphomas. NCI Sponsored International Working Group. J Clin Oncol 17:1244 (1999).
14. Longo D L, DeVita V T, Jr., Duffey P L, et al: Superiority of ProMACE-CytaBOM over ProMACE-MOPP in the treatment of advanced diffuse aggressive lymphoma: results of a prospective randomized trial. J Clin Oncol 9:25-38 (1991).
15. Longo D L, Duffey P L, Gribben J G, et al: Combination chemotherapy followed by an immunotoxin (anti-B4-blocked ricin) in patients with indolent lymphoma: results of a phase 11 study. Cancer J 6:146-50 (2000).
16. A predictive model for aggressive non-Hodgkin's lymphoma. The International Non-Hodgkin's Lymphoma Prognostic Factors Project. N Engl J Med 329:987-94 (1993).
17. Coiffier B, Lepage E, Briere J, et al: CHOP chemotherapy plus rituximab compared with CHOP alone in elderly patients with diffuse large-B-cell lymphoma. N Engl J Med 346:235-42 (2002).
18. Carroll W L, Thielemans K, Dilley J, et al: Mouse×human heterohybridomas as fusionpartners with human B cell tumors. J Immunol Methods 89:61-72 (1986).
19. Jones S E, Schottstaedt M W, Duncan L A, et al: Randomized double-blind prospective trial to evaluate the effects of sargramostim versus placebo in a moderate-dose fluorouracil, doxorubicin, and cyclophosphamide adjuvant chemotherapy program for stage II and III breast cancer. J Clin Oncol 14:2976-83 (1996).
20. Spitler L E, Grossbard M L, Ernstoff M S, et al: Adjuvant therapy of stage III and IV malignant melanoma using granulocyte-macrophage colony-stimulating factor. J Clin Oncol 18:1614-21 (2000).
21. Schulz K F, Chalmers I, Hayes R J, et al: Empirical evidence of bias. Dimensions of methodological quality associated with estimates of treatment effects in controlled trials. JAMA 273:408-12 (1995).
22. Kjaergard L L, Villumsen J, Gluud C: Reported methodologic quality and discrepancies between large and small randomized trials in meta-analyses. Ann Intern Med 135:982-9 (2001).
23. Reitan S K, Hannestad K: A syngeneic idiotype is immunogenic when borne by IgM but tolerogenic when joined to IgG. Eur Immunol 25:1601-8 (1995).
24. Reitan S K, Hannestad K: Immunoglobulin heavy chain constant regions regulate immunity and tolerance to idiotypes of antibody variable regions. Proc Natl Acad Sci USA 99:7588-93 (2002).
25. Baskar S, Kobrin C B, Kwak L W: Autologous lymphoma vaccines induce human T cell responses against multiple, unique epitopes. J Clin Invest 113:1498-510 (2004).
26. Bertinetti C, Zirlik K, Heining-Mikesch K, et al: Phase I trial of a novel intradermal idiotype vaccine in patients with advanced B-cell lymphoma: specific immune responses despite profound immunosuppression. Cancer Res 66:4496-502 (2006).
27. Levy R R M, Leonard J, Vose J, Denney D.: Results of a phase 3 trial evaluating safety and efficacy of specific immunotherapy, recombinant idiotype (Id) conjugated to KLH (Id-KLH) with GM-CSF, compared with non-specific immunotherapy, KLH with GM-CSF, in patients with follicular non Hodgkin's lymphoma (FNHL). Annals of Oncology 19:Suppl 4:iv101-102 (2008).
28. Freedman A, Neelapu S S, Nichols C, et al: Placebo-controlled phase III trial of patient specific immunotherapy with mitumprotimut-T and granulocyte-macrophage colonystimulating factor after rituximab in patients with follicular lymphoma. J Clin Oncol 27:3036-43 (2009).
29. Redfern C H, Guthrie T H, Bessudo A, et al: Phase II trial of idiotype vaccination in previously treated patients with indolent non-Hodgkin's lymphoma resulting in durable clinical responses. J Clin Oncol 24:3107-12 (2006).
30. Fisher R I, LeBlanc M, Press O W, et al: New treatment options have changed the survival of patients with follicular lymphoma. J Clin Oncol 23:8447-52 (2005).
31. Liu Q, Fayad L, Cabanillas F, et al: Improvement of overall and failure-free survival in stage IV follicular lymphoma: 25 years of treatment experience at The University of Texas Md. Anderson Cancer Center. J Clin Oncol 24:1582-9 (2006).

32. Neelapu S S, Kwak L W, Kobrin C B, et al: Vaccine-induced tumor-specific immunity despite severe B-cell depletion in mantle cell lymphoma. Nat Med 11:986-91 (2005).
33. Weng W K, Czerwinski D, Timmerman J, et al: Clinical outcome of lymphoma patients after idiotype vaccination is correlated with humoral immune response and immunoglobulin G Fc receptor genotype. J Clin Oncol 22:4717-24 (2004).
34. Nelson E L, Li X, Hsu F J, et al: Tumor-specific, cytotoxic T-lymphocyte response after idiotype vaccination for B-cell, non-Hodgkin's lymphoma. Blood 88:580-9 (1996).

TABLE 1
Characteristics of randomized patients who
received vaccination (N = 117)

| | Id-vaccine | Control | |
|---|---|---|---|
| | (N = 76) | (N = 41) | |
| Characteristic | No. (%) | No. (%) | P value^{#} |
| Age at enrollment-years | 49.7 ± 9.7* | 51.7 ± 9.1* | 0.146 |
| Male sex | 39 (51.3) | 28 (68.3) | 0.083 |
| White race group | 67 (88.2) | 38 (92.7) | 0.537 |
| ECOG Performance Status | | | 0.222 |
| 0 | 64 (84.2) | 30 (73.2) | |
| 1 | 12 (15.8) | 11 (26.8) | |
| Histology | | | 0.845 |
| FL, grade 1 | 34 (44.7) | 17 (41.5) | |
| FL, grade 2 | 42 (55.3) | 24 (58.5) | |
| IgM isotype | 35 (46.1) | 25 (61.0) | |
| IgG isotype | 40 (52.6) | 15 (36.6) | |
| IgM/IgG isotype | 1 (1.3) | 1 (2.4) | |
| Stage | | | 0.263 |
| II | 2 (2.6) | 1 (2.4)^{a} | |
| III | 29 (38.2) | 10 (24.4)^{b} | |
| IV | 45 (59.2) | 30 (73.2)^{c} | |
| International Prognostic Index | | | 1.000 |
| Low or low intermediate (0-2) | 69 (90.8) | 37 (90.2) | |
| High intermediate or high (3-5) | 7 (9.2) | 4 (9.8) | |
| ≦8 induction chemotherapy cycles | 38 (50.0) | 22 (53.7) | 0.846 |

*Plus-minus values are means ± SD.
^{a}P = 1.000 for the comparison for stage II representation between the two arms.
^{b}P = 0.154 for the comparison for stage III representation between the two arms.
^{c}P = 0.160 for the comparison for stage IV representation between the two arms.
^{#}Comparisons between age groups were performed with non-parametric t-tests using the normal approximation (two-sided Wilcoxon test). Comparisons between groups for the remaining variables were performed using the two-sided Fisher exact test.

TABLE 2
Characteristics of randomized patients
who did not receive vaccination (N = 60)

| | Id-vaccine | Control | |
|---|---|---|---|
| | (N = 42) | (N = 18) | |
| Characteristic | No. (%) | No. (%) | P value^{#} |
| Age at enrollment-years | 49.6 ± 10.3* | 46.6 ± 10.8* | 0.276 |
| Male sex | 21 (50.0) | 7 (38.9) | 0.574 |
| White race group | 37 (88.1) | 14 (77.8) | 0.431 |
| ECOG Performance Status | | | 0.163 |
| 0 | 30 (71.4) | 16 (88.9) | |
| 1 | 11 (26.2) | 1 (5.5) | |
| 2 | 1 (2.4) | 1 (5.5) | |
| Histology | | | 1.000 |
| FL, grade 1 | 20 (47.6) | 8 (44.4) | |
| FL, grade 2 | 22 (52.4) | 10 (55.6) | |
| IgM isotype** | 26 (61.9) | 8 (44.4) | |
| IgG isotype** | 15 (35.7) | 8 (44.4) | |
| IgM/IgG isotype** | 0 (0.0) | 1 (5.6) | |
| IgD isotype** | 1 (2.4) | 1 (5.6) | |
| Stage | | | 0.520 |
| III | 11 (26.2) | 3 (16.7) | |
| IV | 31 (73.8) | 15 (83.3) | |
| International Prognostic Index | | | 1.000 |
| Low or low intermediate (0-2) | 36 (85.7) | 16 (88.9) | |
| High intermediate or high (3-5) | 6 (14.3) | 2 (11.1) | |
| ≦8 induction chemotherapy cycles | 22 (52.4) | 7 (38.9) | 0.405 |

*Plus-minus values are means ± SD.
**Isotypes reflect tumor biopsy isotype as determined by flow cytometry or immunohistochemistry.
^{#}Comparisons between age groups were performed with non-parametric t-tests using the normal approximation (two-sided Wilcoxon test). Comparisons between groups for the remaining variables were performed using the two-sided Fisher exact test.

TABLE 3
Summary of Grade 1 and Grade 2 Adverse Events

| Adverse Event | Id-vaccine | Control | |
|---|---|---|---|
| (Most common ≧10% in | (N = 76) | (N = 41) | |
| either group) | No. (%) | No. (%) | P value^{#} |
| Injection site reaction | 67 (88.2%) | 34 (82.9%) | 0.574 |
| Fatigue | 41 (53.9%) | 16 (39.0%) | 0.175 |
| Myalgia | 35 (46.1%) | 14 (34.1%) | 0.243 |
| Headache | 27 (35.5%) | 12 (29.3%) | 0.543 |
| Arthralgia | 25 (32.9%) | 14 (34.1%) | 1.000 |
| Infection | 16 (21.1%) | 2 (4.9%) | 0.029 |
| Nausea | 16 (21.1%) | 8 (19.5%) | 1.000 |
| Bone pain | 15 (19.7%) | 7 (17.1%) | 0.808 |
| Pruritus | 14 (18.4%) | 9 (22.0%) | 0.635 |
| Non-cardiac chest pain | 13 (17.1%) | 6 (14.6%) | 0.799 |
| Pyrexia | 13 (17.1%) | 5 (12.2%) | 0.596 |
| Dyspepsia | 12 (15.8%) | 3 (7.3%) | 0.253 |
| Flushing | 11 (14.5%) | 4 (9.8%) | 0.571 |
| Influenza like illness | 10 (13.2%) | 5 (12.2%) | 1.000 |
| Pain | 10 (13.2%) | 4 (9.8%) | 0.768 |
| Abdominal pain | 10 (13.2%) | 3 (7.3%) | 0.539 |
| Diarrhea | 10 (13.2%) | 2 (4.9%) | 0.211 |
| Sweating | 9 (11.8%) | 3 (7.3%) | 0.537 |
| Hyperglycaemia | 8 (10.5%) | 1 (2.4%) | 0.158 |

^{#}Comparisons between groups were performed with the two-sided Fisher exact test.

TABLE 4
Comparisons of baseline characteristics for defined cohorts of patients

| | | | | | | Randomized, | | Randomized, | |
|---|---|---|---|---|---|---|---|---|---|
| | | | | Randomized | | vaccinated | | vaccinated | |
| | | Patients | | patients | | patients with | | patients with | |
| | | randomized | | vaccinated | | IgM isotype | | IgG isotype | |
| | Patients | (N = 177) | | (N = 117) | | (N = 60) | | (N = 55) | |
| | enrolled | Id-vaccine | Control | Id-vaccine | Control | Id-vaccine | Control | Id-vaccine | Control |
| | (N = 234) | (N = 118) | (N = 59) | (N = 76) | (N = 41) | (N = 35) | (N = 25) | (N = 40) | (N = 15) |
| Characteristic | No. (%) | No. (%) | No. (%) | No. (%) | No. (%) | No. (%) | No. (%) | No. (%) | No. (%) |
| Age at enrollment - | 49.5 ± 10.4 | 49.8 ± 9.9 | 50.1 ± 9.8 | 49.8 ± 9.7 | 51.7 ± 9.1 | 47.4 ± 8.6 | 51.8 ± 7.3 | 52.1 ± 10.1 | 52.4 ± 11.2 |
| years (mean ± SD) | | | | | | | | | |
| Male sex | 127 (54.3) | 60 (50.8) | 35 (59.3) | 39 (51.3) | 28 (68.3) | 17 (48.6) | 19 (76.0) | 21 (52.5) | 8 (53.3) |
| White race group | 208 (88.9) | 104 (88.1) | 52 (88.1) | 67 (88.2) | 38 (92.7) | 32 (91.4) | 23(92.0) | 35 (87.5) | 14 (93.3) |
| ECOG Performance | | | | | | | | | |
| status | | | | | | | | | |
| 0 | 177 | 94 (79.7) | 46 (78.0) | 64 (84.2) | 30 (73.2) | 30 (85.7) | 20 (80.0) | 33 (82.5) | 9 (60.0) |
| 1 | 54 | 23 (19.5) | 12 (20.3) | 12 (51.8) | 11 (26.8) | 5 (14.3) | 5 (20.0) | 7 (17.5) | 6 (40.0) |
| 2 | 2 | 1 (0.8) | 1 (1.7) | — | — | — | — | — | — |
| Grade not available | 1 | — | — | — | — | — | — | — | — |
| Histology | | | | | | | | | |
| FL, grade 1 | 107 (45.7) | 54 (45.7) | 25 (42.4) | 34 (44.7) | 17 (41.5) | 20 (57.1) | 14 (56.0) | 19 (47.5) | 6 (40.0) |
| FL, grade 2 | 125 (53.4) | 64 (54.2) | 34 (57.6) | 42 (55.3) | 24 (58.5) | 15 (42.9) | 11 (45.9) | 21 (52.5) | 9 (60.0) |
| Not reported | 2 (0.9) | — | — | — | — | — | — | | |
| IgM isotype | | 61 (51.7) | 33 (56.0) | 35 (46.1) | 25 (61.0) | 35 (100.0) | 25 (100.0) | 0 (0.0) | (0.0) |
| IgG isotype | | 55 (46.6) | 23 (39.0) | 40 (52.6) | 15 (36.6) | 0 (0.0) | 0 (0.0) | 40 (100.0) | 15 (100.0) |
| IgM/IgG isotype | | 1 (0.8) | 2 (3.4) | 1 (1.3) | 1 (2.4) | — | — | — | — |
| IgD isotype | | 1 (0.8) | 1 (1.7) | 0 (0.0) | 0 (0.0) | — | — | — | — |
| Stage | | | | | | | | | |
| II | 7 (2.9) | 2 (1.7) | 1 (1.7) | 2 (2.6) | 1 (2.4) | 2 (5.7) | 1 (4.0) | 0 (0.0) | 0 (0.0) |
| III | 62 (26.5) | 40 (33.9) | 13 (22.0) | 29 (38.2) | 10 (24.4) | 9 (25.7) | 7 (28.0) | 19 (47.5) | 3 (20.0) |
| IV | 163 (69.7) | 76 (64.4) | 45 (76.3) | 45 (59.2) | 30 (73.2) | 24 (68.6) | 17 (68.0) | 21 (52.5) | 12 (80.0) |
| Not available | 2 (0.9) | — | — | — | — | — | — | — | — |
| International | | | | | | | | | |
| Prognostic Index | | | | | | | | | |
| Low or low | 205 (87.6) | 105 (89.0) | 53 (89.8) | 69 (90.8) | 37 (90.2) | 33 (94.3) | 22 (88.0) | 35 (87.5) | 14 (93.3) |
| intermediate (0-2) | | | | | | | | | |
| High intermediate or | 28 (12.0) | 13 (11.0) | 6 (10.3) | 7 (9.2) | 4 (9.8) | 2 (5.7) | 3 (12.0) | 5 (12.5) | 1 (6.67) |
| high (3-5) | | | | | | | | | |
| Not available | 1 (0.4) | — | — | — | — | — | — | — | — |
| <8 chemotherapy | 89 (38.0)* | 60 (50.8) | 29 (49.1) | 38 (50.0) | 22 (53.7) | 18 (51.4) | 12 (48.0) | 19 (47.5) | 9 (60.0) |
| cycles | | | | | | | | | |

*Data is based on the 177 randomized patients.
^{#}Comparisons between age groups were performed with non-parametric t-tests using the normal approximation (two-sided Wilcoxon test). Comparisons between groups for the remaining variables were performed using the two-sided Fisher exact test. No P values for reached statistical significance (p < 0.05).

TABLE 5
Accrual Rate by Year

| Year | Patients Enrolled No. (%) |
|---|---|
| 2000 | 39 (16.7) |
| 2001 | 35 (14.9) |
| 2002 | 51 (21.8) |
| 2003 | 30 (12.8) |
| 2004 | 23 (9.8) |
| 2005 | 22 (9.4) |
| 2006 | 20 (8.5) |
| 2007 | 14 (5.9) |
| Total | 234 |

TABLE 6
Distribution of tumor Ig isotype by treatment arm for
randomized and vaccinated patients (N = 117)

| | | Vaccine | Isotype Analysis | |
|---|---|---|---|---|
| Treatment Arm | Biopsy Isotype | Isotype | Group | N |
| Id-KLH | IgM | IgM | IgM | 29 |
| Id-KLH | IgM/IgD | IgM | IgM | 4 |
| Id-KLH | IgM/IgG | IgM | IgM | 1 |
| Id-KLH | IgM | KLH-KLH | IgM | 1 |
| Id-KLH | IgG | IgG | IgG | 35 |
| Id-KLH | IgM/IgG | IgG | IgG | 2 |
| Id-KLH | IgG | KLH-KLH | IgG | 3 |
| Id-KLH | IgM/IgG | KLH-KLH | Excluded | 1 |
| Control | IgM | KLH-KLH | IgM | 23 |
| Control | IgM/IgD | KLH-KLH | IgM | 1 |
| Control | IgM/IgA | KLH-KLH | IgM | 1 |
| Control | IgG | KLH-KLH | IgG | 13 |
| Control | IgG/IgA | KLH-KLH | IgG | 2 |
| Control | IgM/IgG | KLH-KLH | Excluded | 1 |

TABLE 7
Summary of Grade 3 and Grade 4 Adverse Events

| | Id-vaccine | Control |
|---|---|---|
| | (N = 76) | (N = 41) |
| Adverse Event | No. (%) | No. (%) |
| Vomiting | 1 (1.3%) | 1 (2.4%) |
| Urticaria | 1 (1.3%) | 1 (2.4%) |
| Headache | 1 (1.3%) | 1 (2.4%) |
| Osteonecrosis | 1 (1.3%) | 0 (0.0%) |
| Fatigue | 1 (1.3%) | 0 (0.0%) |
| Injection site reaction | 1 (1.3%) | 0 (0.0%) |
| Myalgia | 1 (1.3%) | 0 (0.0%) |
| Diarrhea | 1 (1.3%) | 0 (0.0%) |
| Non-cardiac chest pain | 1 (1.3%) | 0 (0.0%) |
| Cerebral ischemia | 1 (1.3%) | 0 (0.0%) |
| Myocardial ischemia | 1 (1.3%) | 0 (0.0%) |
| Hypertension | 1 (1.3%) | 0 (0.0%) |
| Abdominal pain | 1 (1.3%) | 0 (0.0%) |
| Dyspepsia | 1 (1.3%) | 0 (0.0%) |
| Erythema multiforme | 1 (1.3%) | 0 (0.0%) |
| Acute myeloid leukemia | 1 (1.3%) | 0 (0.0%) |
| Induration | 1 (1.3%) | 0 (0.0%) |
| Dizziness | 0 (0.0%) | 1 (2.4%) |
| Arthralgia | 0 (0.0%) | 1 (2.4%) |
| Compression fracture | 0 (0.0%) | 1 (2.4%) |
| Dyspnea | 0 (0.0%) | 1 (2.4%) |
| Pain | 0 (0.0%) | 1 (2.4%) |
| Herpes zoster | 0 (0.0%) | 1 (2.4%) |
| Arrhythmia | 0 (0.0%) | 1 (2.4%) |
| Squamous cell carcinoma of | 0 (0.0%) | 1 (2.4%) |
| skin | | |
| Cystitis interstitial | 0 (0.0%) | 1 (2.4%) |
| Intervertebral disc protrusion | 0 (0.0%) | 1 (2.4%) |
| Total^{#} | 17 | 13 |

^{#}Between groups comparison for the overall rate of grade 3-4 adverse events was performed with the two-sided Fisher exact statistic using the total number of vaccinations administered for all patients on each arm (P = 0.331).

TABLE 8
comparison of NCI Phase 2 and Randomized phase III clinical trials with idiotype vaccine in follicular
lymphoma

| | NCI Phase 2^{3,4} | NCI/Biovest | Genitope⁵ | Favrille⁶ |
|---|---|---|---|---|
| | (NCT00001512) | (NCT00091676) | (NCT00017290) | (NCT00089115) |
| Id protein in vaccine | Native protein from | Native protein from | Recombinant protein | Recombinant protein |
| formulation | heterohybridoma | heterohybridoma | from mammalian cell | from Sf9 (insect) cell |
| | | | line | line |
| Isotype of Id-vaccine | IgM or IgG | IgM or IgG | IgG | IgG |
| | (tumor-matched) | (tumor-matched) | | |
| Induction therapy | PACE | PACE | CVP | Rituximab |
| Prerequisite for | CR/CRu | CR/CRu | CR/CRu/PR | CR/CRu/PR/SD |
| vaccination | | | | |
| Randomization | Open-label | 2:1 | 2:1 | 1:1 |
| Stratification | Not applicable | IPI score 0-2 vs. 3, 4 | Not reported | Treatment-naive vs. |
| | | <8 vs. ≧8 cycles | | relapsed |
| | | | | CR/CRu/PR vs. SD |
| Primary endpoint | Induction of immune | DFS | Progression-free | Time to progression |
| | responses and | | survival | |
| | molecular remissions | | | |
| Clinical outcome with Id- | 75% antibody | Significant | No improvement | No improvement |
| vaccine | responses and 95% T- | improvement in DFS | | |
| | cell responses; 45% | | | |
| | remain in CR after | | | |
| | median follow-up of | | | |
| | 9.2 years | | | |

NCI, National Cancer Institute;
FL, follicular lymphoma;
PACE, prednisone, doxorubicin, cyclophosphamide, etoposide;
CVP, cyclophosphamide, vincristine, prednisone;
CR, complete response,
CRu, complete response unconfirmed;
PR, partial response;
SD, stable disease,
IPI, Internatonal Prognostic Index;
ID, idiotype,;
DFS, disease-free survival.

What is claimed is:

1. A method for directing an immune response to an epitope from an antigen in a subject, comprising:
a) sensitizing the subject to the epitope, comprising co-administering the epitope and an immunoglobulin M (IgM) constant region (IgM Fc region) to the subject; or
b) tolerizing the subject to the epitope, comprising co-administering the epitope and an immunoglobulin G (IgG) constant region (IgG Fc region) to the subject.

2. The method of claim 1, wherein said sensitizing of (a) is carried out, wherein said sensitizing of (a) comprises administering a fusion polypeptide comprising the epitope and the IgM Fc region.

3. The method of claim 1, wherein said sensitizing of (a) is carried out, and wherein said sensitizing of (a) comprises administering a nucleic acid molecule encoding the epitope and the IgM Fc region, and wherein the nucleic acid molecule is expressed to produce the epitope and the IgM Fc region separately or as a fusion polypeptide.

4. The method of claim 1, wherein said sensitizing of (a) is carried out, and wherein said sensitizing of (a) comprises co-administering the epitope and the IgM Fc separately, in separate formulations or in the same formulation.

5. The method of claim 1, wherein said sensitizing of (a) is carried out, further comprising administering at least one immune adjuvant (for example, granulocyte-monocyte colony stimulating fragment (GM-CSF) or bovine serum albumin (BSA)) before, simultaneously with, or after co-administration of the epitope and IgM Fc region.

6. The method of claim 1, wherein said sensitizing of (a) is carried out, and wherein the epitope and the IgM Fc region are administered in conjunction with a carrier protein.

7. The method of claim 1, wherein said tolerizing of (b) is carried out, and wherein said tolerizing of (b) comprises suppression of effector T cell response, suppression of helper T cell response, suppression of B cell response, or suppression of two or more of the foregoing, in the subject.

8. The method of claim 1, wherein said tolerizing of (b) is carried out, and wherein said tolerizing of (b) comprises administering a fusion polypeptide comprising the epitope and the IgG Fc region.

9. The method of claim 1, wherein said tolerizing of (b) is carried out, and wherein said tolerizing of (b) comprises administering a nucleic acid molecule encoding the epitope and the IgG Fc region, and wherein the nucleic acid molecule is expressed to produce the epitope and the IgG Fc region separately or as a fusion polypeptide.

10. The method of claim 1, wherein said tolerizing of (b) is carried out, and wherein said tolerizing of (b) comprises co-administering the epitope and the IgG Fc separately, in separate formulations or in the same formulation.

11-24. (canceled)

25. The method of claim 1, wherein the antigen is an immunoglobulin expressed by a B-cell malignancy.

26-34. (canceled)

35. The method of claim 1, wherein the antigen is an immunoglobulin expressed by a B-cell malignancy, and wherein the immunoglobulin isotype or isotypes exhibited by the malignancy represents an immunoglobulin that is present on the malignant cell (surface), within the malignant cell, secreted by the malignancy or is found in the subject's blood, or any combination of two or more of the foregoing.

36. The method of claim 35, wherein the immunoglobulin isotype or isotypes exhibited by the malignancy is predetermined by at least one of the following:
(a) obtaining a tumor, tissue or blood sample from the subject by biopsy, needle aspiration, or apheresis;
(b) obtaining a sample of lymph node tissue, extra-nodal tissue, spleen, bone marrow, or blood; and
(c) flow cytometry, immunofluoroescence, sequencing of heavy chain constant region, or immunoblot.

37-39. (canceled)

40. A composition of matter comprising:
(a) a composition comprising an epitope; and an immunoglobulin M (IgM) constant region (IgM Fc region) or an immunoglobulin G (IgG) constant region (IgG Fc region; or
(b) a kit for sensitizing a subject to an epitope of an antigen, wherein said kit comprises at least one IgM Fc region and printed instructions for sensitizing a subject to an epitope using said IgM Fc region; or
(c) a kit for sensitizing or tolerizing a subject to an epitope, wherein said kit comprises at least one IgM Fc region, at least one IgG Fc region, printed instructions for sensitizing a subject to an epitope using the IgM Fc region, and printed instructions for tolerizing a subject to an epitope using the IgM Fc region; or
(d) a kit for detecting the T-regulatory (T-reg) cell response before, during, and after administration of a T-reg inhibitor prior to administration of an epitope and an immunoglobulin M (IgM) constant region (IgM Fc region), wherein said kit comprises one or more reagents for assessing T-reg cell response in a subject; and printed instructions for making the assessment.

41-47. (canceled)

48. The composition of claim 40, wherein said composition comprises (a), and wherein said composition comprises said epitope and said IgG Fc region, and wherein said composition further comprises an immunosuppressive agent.

49-59. (canceled)

60. A kit for tolerizing a subject to an epitope, wherein said kit comprises at least one IgG Fc region and printed instructions for tolerizing a subject to an epitope.

61. The tolerizing kit of claim 60, further comprising an epitope, adjuvant, carrier protein, an assay for T-regulatory cell number and/or activity, an assay for immune response, or any combination of two or more of the foregoing.

62. The tolerizing kit of claim 60, wherein the epitope is of an antigen that is an immunoglobulin expressed by a B-cell malignancy.

63. The tolerizing kit of claim 60, wherein the epitope is of an antigen that is not an immunoglobulin.

64. The tolerizing kit of claim 60, wherein the epitope is of an antigen that is not an immunoglobulin expressed by a B-cell malignancy.

65-78. (canceled)
